(19) United States
(12) Patent Application Publication
(10) Pub. No.: US 2015/0133662 A1
(43) Pub. Date: May 14, 2015

(54) NOVEL ORGANIC ELECTROLUMINESCENCE COMPOUNDS AND ORGANIC ELECTROLUMINESCENCE DEVICE CONTAINING THE SAME

(71) Applicant: Rohm and Haas Electronic Materials Korea Ltd., Cheonan (KR)

(72) Inventors: Hee-Choon Ahn, Suwon (KR); Young-Jun Cho, Seongnam (KR); Bong-Ok Kim, Seoul (KR); Nam-Kyun Kim, Yongin (KR); Jong-Seok Ku, Hwaseong (KR); Hyuck-Joo Kwon, Seoul (KR); Doo-Hyeon Moon, Hwaseong (KR)

(21) Appl. No.: 14/400,420

(22) PCT Filed: Jun. 12, 2013
(86) PCT No.: PCT/KR2013/005171

§ 371 Date: Nov. 11, 2014

(30) Foreign Application Priority Data
Jun. 13, 2012 (KR) 10-2012-0063068

(51) Int. Cl. H01L 51/00; C07D 403/04; C07D 403/14

(52) U.S. Cl. CPC H01L 51/0072; C07D 403/14; C07D 403/04; H01L 51/0061; H01L 51/5012
USPC 544/284

(57) ABSTRACT

The present invention relates to novel organic electroluminescent compounds and an organic electroluminescent device comprising the same. The organic electroluminescent compounds according to the present invention have high luminescent efficiency and a long lifespan, and thus an organic electroluminescent device having a long operating lifespan can be prepared by using the organic electroluminescent compounds according to the present invention.

### TECHNICAL FIELD

The present invention relates to novel organic electroluminescent compounds and organic electroluminescent device comprising the same.

### BACKGROUND ART

An electroluminescent (EL) device is a self-light-emitting device with the advantage of providing a wider viewing angle, a greater contrast ratio, and a faster response time. An organic EL device was first developed by Eastman Kodak, by using small aromatic diamine molecules and aluminum complexes as material for forming a light-emitting layer [see Appl. Phys. Lett. 51, 913, 1987].

The most important factor determining luminescent efficiency in an organic EL device is the light-emitting material. Until now, fluorescent materials have been widely used as a light-emitting material. However, in view of electroluminescent mechanisms, developing phosphorescent materials is one of the best methods to theoretically enhance the luminescent efficiency by four (4) times compared to fluorescent materials. Iridium(III) complexes have been widely known as phosphorescent materials, including bis(2-(2′-benzothienyl)-pyridinato-N,C3′)iridium (acetylacetonate) ((acac)Ir(btp)₂), tris(2-phenylpyridine)iridium (Ir(ppy)₃) and bis(4,6-difluorophenylpyridinato-N,C2)picolinato iridium (Firpic) as red, green and blue materials, respectively. Especially, many phosphorescent materials are being researched in Japan, Europe and U.S.A. recently.

Until now, 4,4′-N,N′-dicarbazol-biphenyl (CBP) is the most widely known host material for phosphorescent substances. Further, high performance organic EL devices using bathocuproine (BCP), aluminum(III)bis(2-methyl-8-quinolinate)(4-phenylphenolate) (BAIq), etc., for a hole blocking layer are known, and Pioneer (Japan) et al. developed a high performance organic EL device employing a derivative of BAIq as a host material.

Though these phosphorescent host materials provide good light-emitting characteristics, they have the following disadvantages: (1) Due to their low glass transition temperatures and poor thermal stability, their degradation may occur during a high-temperature deposition process in a vacuum. (2) The power efficiency of an organic EL device is given by [(π/voltage)×current efficiency], and thus the power efficiency is inversely proportional to voltage. An organic EL device comprising phosphorescent host materials provides higher current efficiency (cd/A) but has a higher driving voltage than one comprising fluorescent host materials. Thus, the organic EL device using conventional phosphorescent materials has no advantage in terms of power efficiency (Im/W). (3) The operating lifespan and luminescent efficiency of the organic EL device using phosphorescent host materials are not satisfactory.

International Publication No. WO 2006/049013 discloses a compound for an organic EL device, wherein the compound has a nitrogen-containing 3-membered heterocyclic ring to which a 2-membered heterocyclic ring is directly or via a linking group bonded. However, the device comprising the compound is not satisfactory in terms of an operating lifespan and luminescent efficiency.

### DISCLOSURE OF THE INVENTION

### Problems to be Solved

The objective of the present invention is to provide an organic electroluminescenct compound imparting higher luminescent efficiency and a longer operating lifespan to a device over conventional materials in order to overcome said problems; and an organic electroluminescent device having high efficiency and a long lifespan, comprising the organic electroluminescent compound of the present invention as a light-emitting material.

### Solution to Problems

The present inventors found that the above objective can be achieved by a compound represented by the following formula 1:

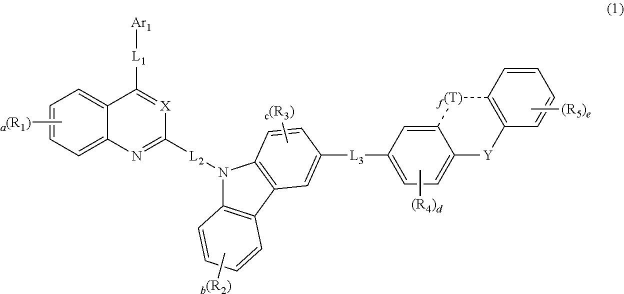

wherein

X represents CH or N;

L₁ and L₂ each independently represent a single bond, a substituted or unsubstituted 5- to 30-membered heteroarylene group, or a substituted or unsubstituted (C6-C30)arylene group;

L₃ represents a single bond, a substituted or unsubstituted (C1-C30)alkylene group, a substituted or unsubstituted (C6-C30)arylene group, or a substituted or unsubstituted 3- to 30-membered heteroarylene group;

Y represents —O—, —S—, —CR₁₁R₁₂— or —NR₁₃—;

T represents a chemical bond;

Ar₁ represents hydrogen, a halogen, deuterium, a substituted or unsubstituted 5- to 30-membered heteroaryl group, a substituted or unsubstituted (C6-C30)aryl group, or a substituted or unsubstituted (C1-C30)alkyl group;

R₁ to R₅ each independently represent hydrogen, deuterium, a halogen, a substituted or unsubstituted (C1-C30)alkyl group, a substituted or unsubstituted (C6-C30)aryl group, a substituted or unsubstituted 3- to 30-membered heteroaryl group, a substituted or unsubstituted (C3-C30)cycloalkyl group, a substituted or unsubstituted 5- to 7-membered heterocycloalkyl group, a substituted or unsubstituted (C6-C30)aryl(C1-C30)alkyl group, a substituted or unsubstituted (C6-C30)aryl group which is fused with at least one (C3-C30) aliphatic ring, a 5- to 7-membered heterocycloalkyl group which is fused with at least one substituted or unsubstituted (C6-C30) aromatic ring, a (C3-C30)cycloalkyl group which is fused with at least one substituted or unsubstituted (C6-C30) aromatic ring, —NR₁₄R₁₅, —SiR₁₆R₁₇R₁₈, —SR₁₉, —OR₂₀, a (C2-C30)alkenyl group, a (C2-C30)alkynyl group, a cyano group, a nitro group, or a hydroxyl group;

R₁₁ to R₂₀ are as defined in R₁ to R₅;

a, b and e each independently represent an integer of 1 to 4; where a, b or e is an integer of 2 or more, each of R₁, each of R₂ or each of R₅ is the same or different;

c and d each independently represent an integer of 1 to 3; where c or d is an integer of 2 or more, each of R₃ or each of R₄ is the same or different;

f represents an integer of 0 or 1; where f is 0, Y represents —NR₁₃—, wherein R₁₃ may be linked to R₅ to form a mono- or polycyclic, (C5-C30) alicyclic or aromatic ring whose carbon atom(s) may be replaced with at least one hetero atom selected from nitrogen, oxygen and sulfur; and

the heterocycloalkyl group and heteroaryl(ene) group contain at least one hetero atom selected from B, N, O, S, P(═O), Si and P.

### Effects of the Invention

The organic electroluminescent compounds according to the present invention have high luminescent efficiency and a long lifespan. Therefore, an organic electroluminescent device comprising the compounds according to the present invention has a long operating lifespan.

### EMBODIMENTS OF THE INVENTION

Hereinafter, the present invention will be described in detail. However, the following description is intended to explain the invention, and is not meant in any way to restrict the scope of the invention.

The present invention relates to an organic electroluminescent compound represented by the formula 1, an organic electroluminescent material comprising the organic electroluminescent compound, and an organic electroluminescent device comprising the material.

In formula 1, preferably, L₁ and L₂ each independently represent a single bond, a substituted or unsubstituted 5- to 15-membered heteroarylene group, or a substituted or unsubstituted (C6-C15)arylene group and more preferably a single bond, an unsubstituted 5- to 15-membered heteroarylene group, an unsubstituted (C6-C15)arylene group, or a (C6-C15)arylene group which is substituted with a (C1-C6) alkyl group. More specifically, L₁ and L₂ may be each independently selected from the group consisting of a single bond, phenylene, naphthylene, biphenylene, terphenylene, anthrylene, indenylene, fluorenylene, phenanthrylene, triphenylenylene, pyrenylene, perylenylene, chrysenylene, naphthacenylene, fluoranthenylene, furylene, thiophenylene, pyrrolylene, imidazolylene, pyrazolylene, thiazolylene, thiadiazolylene, isothiazolylene, isoxazolylene, oxazolylene, oxadiazolylene, triazinylene, tetrazinylene, triazolylene, tetrazolylene, furazanylene, pyridylene, pyrazinylene, pyrimidinylene, pyridazinylene, benzofuranylene, benzothiophenylene, isobenzofuranylene, benzoimidazolylene, benzothiazolylene, benzoisothiazolylene, benzoisoxazolylene, benzoxazolylene, isoindolylene, indolylene, indazolylene, benzothiadiazolylene, quinolylene, isoquinolylene, cinolynylene, quinazolinylene, quinoxalynylene, carbazolylene, phenanthrydinylene, benzodioxolylene, dibenzofuranylene and dibenzothiophenylene.

In formula 1, L₃ preferably represents a single bond, or a substituted or unsubstituted (C6-C15)arylene group and more preferably a single bond or an unsubstituted (C6-C15)arylene group.

In formula 1, Y represents —O—, —S—, —CR₁₁R₁₂— or —NR₁₃—, in which preferably R₁₁ and R₁₂ each independently represent a substituted or unsubstituted (C1-C10)alkyl group and more preferably an unsubstituted (C1-C10)alkyl group, and R₁₃ preferably represents a substituted or unsubstituted (C6-C15)aryl group, or a substituted or unsubstituted 5- to 15-membered heteroaryl group and more preferably a (C6-C15)aryl group which is unsubstituted or substituted with deuterium, a halogen, a (C1-C6)alkyl group or a (C6-C15)aryl group, or a 5- to 15-membered heteroaryl group which is substituted with a (C6-C15)aryl group.

In formula 1, T preferably represents a single bond.

In formula 1, Ar₁ preferably represents hydrogen, a substituted or unsubstituted 5- to 20-membered heteroaryl group, a substituted or unsubstituted (C6-C20)aryl group, or a substituted or unsubstituted (C1-C10)alkyl group and more preferably hydrogen; a 5- to 20-membered heteroaryl group which is unsubstituted or substituted with a (C6-C15)aryl group; a (C6-C20)aryl group which is unsubstituted or substituted with deuterium, a halogen, a (C1-C6)alkyl group, a (C6-C15)aryl group or a 5- to 15-membered heteroaryl group; or a (C1-C10)alkyl group which is unsubstituted or substituted with a (C1-C6)alkyl group.

In formula 1, preferably, R₁ to R₅ each independently represent hydrogen, a substituted or unsubstituted (C6-C15)aryl group, or a substituted or unsubstituted 5- to 15-membered heteroaryl group and more preferably hydrogen, an unsubstituted (C6-C15)aryl group or an unsubstituted 5- to 15-membered heteroaryl group.

Herein, “(C1-C30)alkyl(ene)” is meant to be a linear or branched alkyl(ene) having 1 to 30 carbon atoms, in which the number of carbon atoms is preferably 1 to 20, more preferably 1 to 10, and includes methyl, ethyl, n-propyl, isopropyl, n-butyl, isobutyl, tert-butyl, etc. “(C2-C30) alkenyl” is meant to be a linear or branched alkenyl having 2 to 30 carbon atoms, in which the number of carbon atoms is preferably 2 to 20, more preferably 2 to 10, and includes vinyl, 1-propenyl, 2-propenyl, 1-butenyl, 2-butenyl, 3-butenyl, 2-methylbut-2-enyl, etc. “(C2-C30)alkynyl” is a linear or branched alkynyl having 2 to 30 carbon atoms, in which the number of carbon atoms is preferably 2 to 20, more preferably 2 to 10, and includes ethynyl, 1-propynyl, 2-propynyl, 1-butynyl, 2-butynyl, 3-butynyl, 1-methylpent-2-ynyl, etc. “(C3-C30)cycloalkyl” is a mono- or polycyclic hydrocarbon having 3 to 30 carbon atoms, in which the number of carbon atoms is preferably 3 to 20, more preferably 3 to 7, and includes cyclopropyl, cyclobutyl, cyclopentyl, cyclohexyl, etc. “3- to 7-membered heterocycloalkyl” is a cycloalkyl having at least one heteroatom selected from B, N, O, S, P(═O), Si and P, preferably O, S and N, and 3 to 7 ring backbone atoms, and includes tetrahydrofurane, pyrrolidine, thiolan, tetrahydropyran, etc. “(C6-C30)aryl(ene)” is a monocyclic or fused ring derived from an aromatic hydrocarbon having 6 to 30 carbon atoms, in which the number of carbon atoms is preferably 6 to 20, more preferably 6 to 15, and includes phenyl, biphenyl, terphenyl, naphthyl, fluorenyl, phenanthrenyl, anthracenyl, indenyl, triphenylenyl, pyrenyl, tetracenyl, perylenyl, chrysenyl, naphthacenyl, fluoranthenyl, etc. “3- to 30-membered heteroaryl(ene)” is an aryl group having at least one, preferably 1 to 4 heteroatom selected from the group consisting of B, N, O, S, P(═O), Si and P, and 3 to 30 ring backbone atoms; is a monocyclic ring, or a fused ring condensed with at least one benzene ring; has preferably 5 to 20, more preferably 5 to 15 ring backbone atoms; may be partially saturated; may be one formed by linking at least one heteroaryl or aryl group to a heteroaryl group via a single bond(s); and includes a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, benzoimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, phenoxazinyl, phenanthridinyl, benzodioxolyl, etc. Further, “halogen” includes F, Cl, Br and I.

Herein, “substituted” in the expression “substituted or unsubstituted” means that a hydrogen atom in a certain functional group is replaced with another atom or group, i.e., a substituent. Substituents of the substituted alkyl(ene) group, the substituted aryl(ene) group, the substituted heteroaryl(ene) group, the substituted cycloalkyl group, and the substituted heterocycloalkyl group in formula 1 each independently are at least one selected from the group consisting of deuterium; a halogen; a (C1-C30)alkyl group which is unsubstituted or substituted with a halogen; a (C6-C30)aryl group; a 3- to 30-membered heteroaryl group which is unsubstituted or substituted with a (C6-C30)aryl group; a 5- to 7-membered heterocycloalkyl group; a 5- to 7-membered heterocycloalkyl group which is fused with at least one (C6-C30) aromatic ring; a (C3-C30)cycloalkyl group; a (C6-C30)cycloalkyl group which is fused with at least one (C6-C30) aromatic ring; RₐR_{b}R_{c}Si—; a (C2-C30)alkenyl group; a (C2-C30)alkynyl group; a cyano group; a carbazolyl group; —NR_{d}Rₑ; —BR_{f}R_{g}; —PRₕRᵢ; —P(═O)RⱼRₖ; a (C6-C30)aryl(C1-C30)alkyl group; (C1-C30)alkyl(C6-C30)aryl group; RₗZ—; RₘC(═O)—; RₘC(═O)O—; a carboxyl group; a nitro group; and a hydroxyl group, in which Rₐ to Rₗ each independently represent a (C1-C30)alkyl group, a (C6-C30)aryl group or a 3- to 30-membered heteroaryl group, or are linked to an adjacent substituent(s) to form a mono- or polycyclic, (C5-C30) alicyclic or aromatic ring whose carbon atom(s) may be replaced with at least one hetero atom selected from nitrogen, oxygen and sulfur, Z represents S or O, and Rₘ represents a (C1-C30)alkyl group, a (C1-C30)alkoxy group, a (C6-C30)aryl group or a (C6-C30)aryloxy group.

The organic electroluminescent compounds according to the present invention include the following compounds:

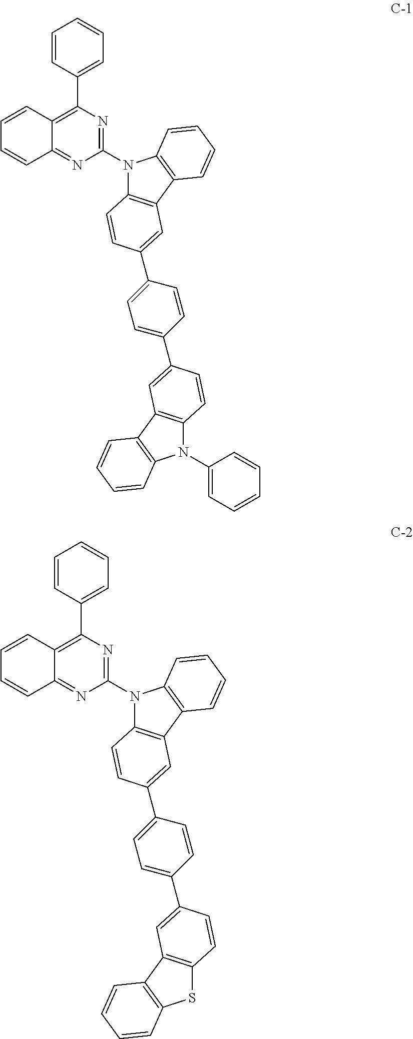 [SMILES: c1ccc(-c2nc(-n3c4ccccc4c4cc(-c5ccc(-c6ccc7c(c6)c6ccccc6n7-c6ccccc6)cc5)ccc43)nc3ccccc23)cc1 ; c1ccc(-c2nc(-n3c4ccccc4c4cc(-c5ccc(-c6ccc7sc8ccccc8c7c6)cc5)ccc43)nc3ccccc23)cc1]

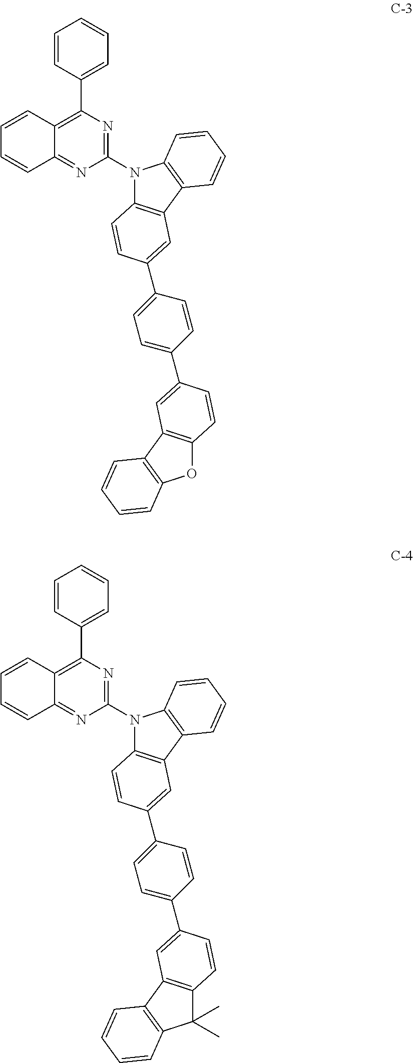 [SMILES: c1ccc(-c2nc(-n3c4ccccc4c4cc(-c5ccc(-c6ccc7oc8ccccc8c7c6)cc5)ccc43)nc3ccccc23)cc1 ; CC1(C)c2ccccc2-c2cc(-c3ccc(-c4ccc5c(c4)c4ccccc4n5-c4nc(-c5ccccc5)c5ccccc5n4)cc3)ccc21]

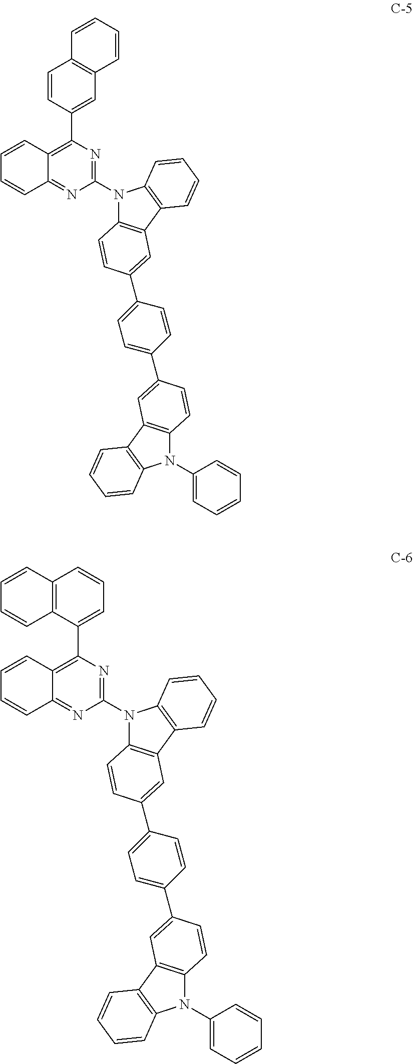 [SMILES: c1ccc(-n2c3ccccc3c3cc(-c4ccc(-c5ccc6c(c5)c5ccccc5n6-c5nc(-c6ccc7ccccc7c6)c6ccccc6n5)cc4)ccc32)cc1 ; c1ccc(-n2c3ccccc3c3cc(-c4ccc(-c5ccc6c(c5)c5ccccc5n6-c5nc(-c6cccc7ccccc67)c6ccccc6n5)cc4)ccc32)cc1]

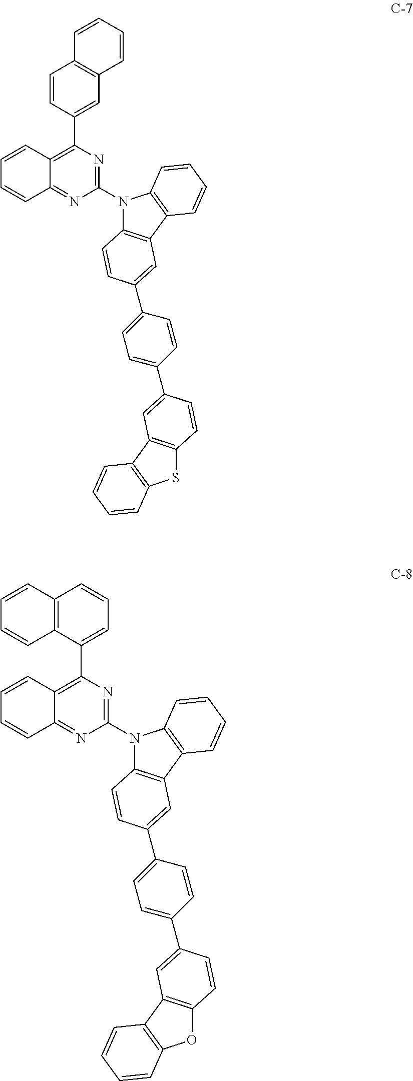 [SMILES: c1ccc2cc(-c3nc(-n4c5ccccc5c5cc(-c6ccc(-c7ccc8sc9ccccc9c8c7)cc6)ccc54)nc4ccccc34)ccc2c1 ; c1ccc2c(-c3nc(-n4c5ccccc5c5cc(-c6ccc(-c7ccc8oc9ccccc9c8c7)cc6)ccc54)nc4ccccc34)cccc2c1]

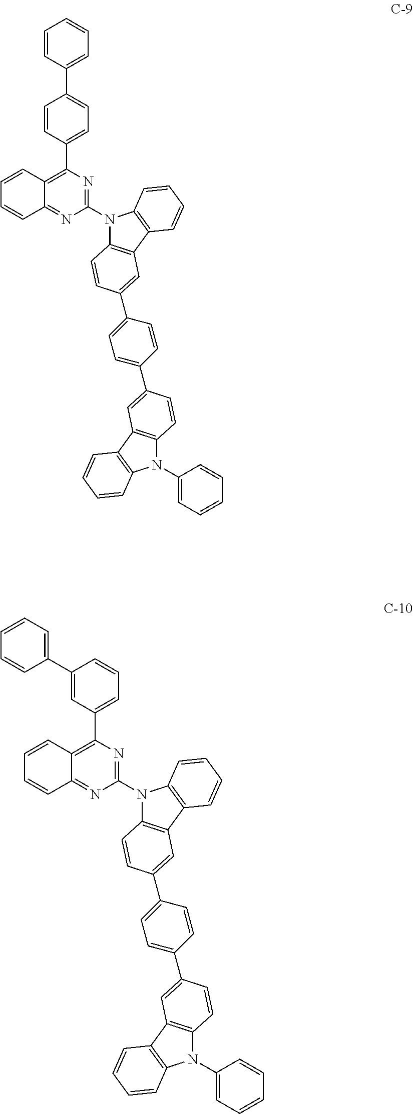 [SMILES: c1ccc(-c2ccc(-c3nc(-n4c5ccccc5c5cc(-c6ccc(-c7ccc8c(c7)c7ccccc7n8-c7ccccc7)cc6)ccc54)nc4ccccc34)cc2)cc1 ; c1ccc(-c2cccc(-c3nc(-n4c5ccccc5c5cc(-c6ccc(-c7ccc8c(c7)c7ccccc7n8-c7ccccc7)cc6)ccc54)nc4ccccc34)c2)cc1]

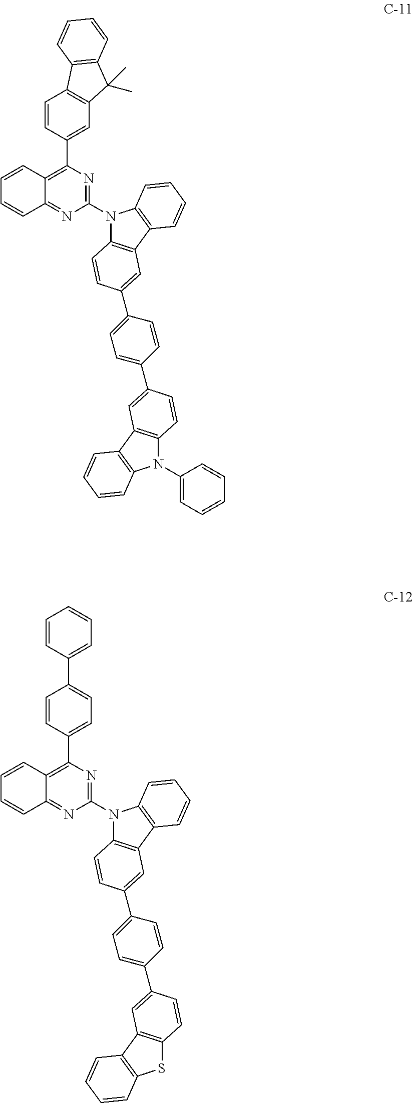 [SMILES: CC1(C)c2ccccc2-c2ccc(-c3nc(-n4c5ccccc5c5cc(-c6ccc(-c7ccc8c(c7)c7ccccc7n8-c7ccccc7)cc6)ccc54)nc4ccccc34)cc21 ; c1ccc(-c2ccc(-c3nc(-n4c5ccccc5c5cc(-c6ccc(-c7ccc8sc9ccccc9c8c7)cc6)ccc54)nc4ccccc34)cc2)cc1]

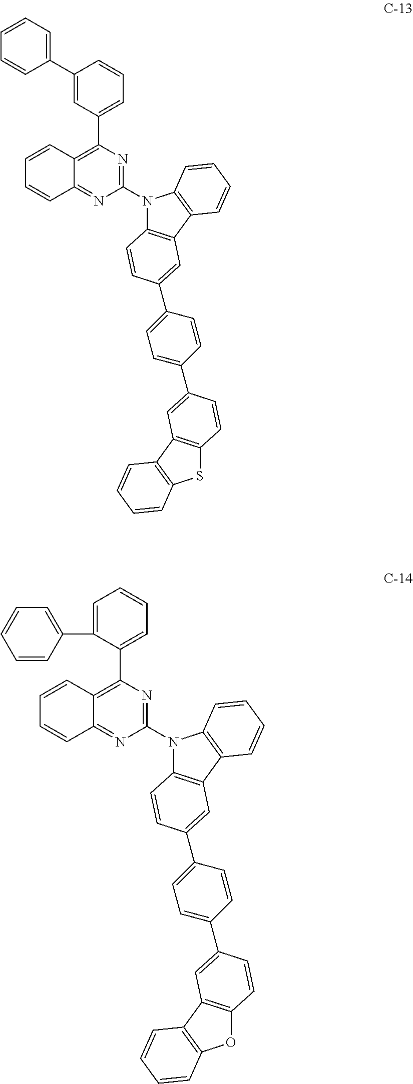 [SMILES: c1ccc(-c2cccc(-c3nc(-n4c5ccccc5c5cc(-c6ccc(-c7ccc8sc9ccccc9c8c7)cc6)ccc54)nc4ccccc34)c2)cc1 ; c1ccc(-c2ccccc2-c2nc(-n3c4ccccc4c4cc(-c5ccc(-c6ccc7oc8ccccc8c7c6)cc5)ccc43)nc3ccccc23)cc1]

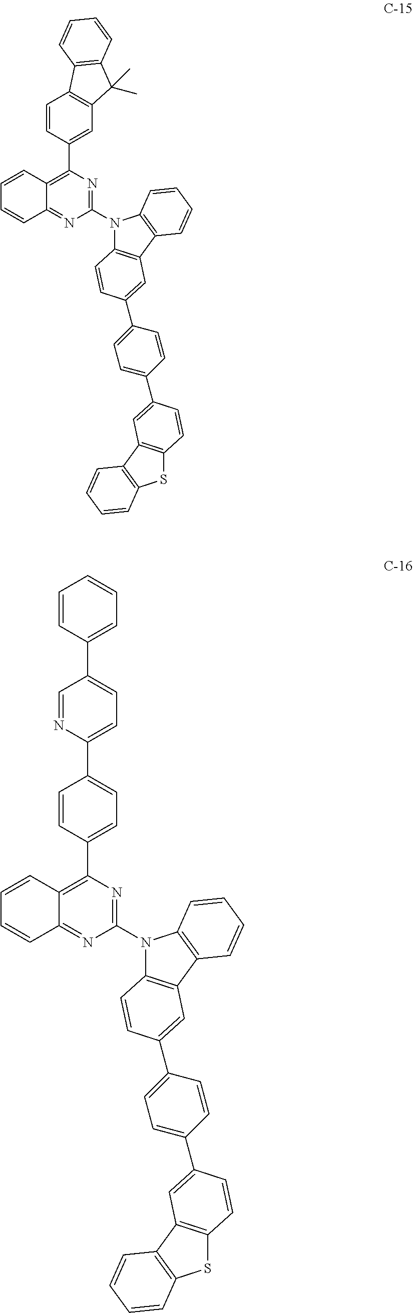 [SMILES: CC1(C)c2ccccc2-c2ccc(-c3nc(-n4c5ccccc5c5cc(-c6ccc(-c7ccc8sc9ccccc9c8c7)cc6)ccc54)nc4ccccc34)cc21 ; c1ccc(-c2ccc(-c3ccc(-c4nc(-n5c6ccccc6c6cc(-c7ccc(-c8ccc9sc%10ccccc%10c9c8)cc7)ccc65)nc5ccccc45)cc3)nc2)cc1]

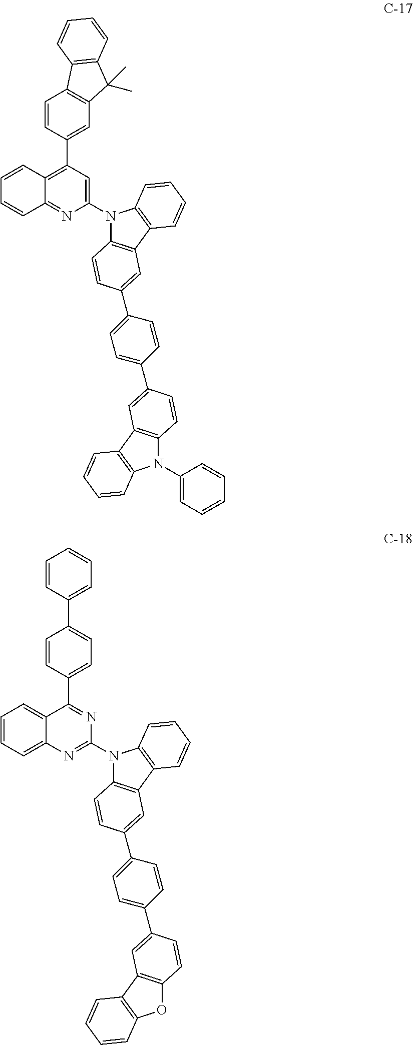 [SMILES: CC1(C)c2ccccc2-c2ccc(-c3cc(-n4c5ccccc5c5cc(-c6ccc(-c7ccc8c(c7)c7ccccc7n8-c7ccccc7)cc6)ccc54)nc4ccccc34)cc21 ; c1ccc(-c2ccc(-c3nc(-n4c5ccccc5c5cc(-c6ccc(-c7ccc8oc9ccccc9c8c7)cc6)ccc54)nc4ccccc34)cc2)cc1]

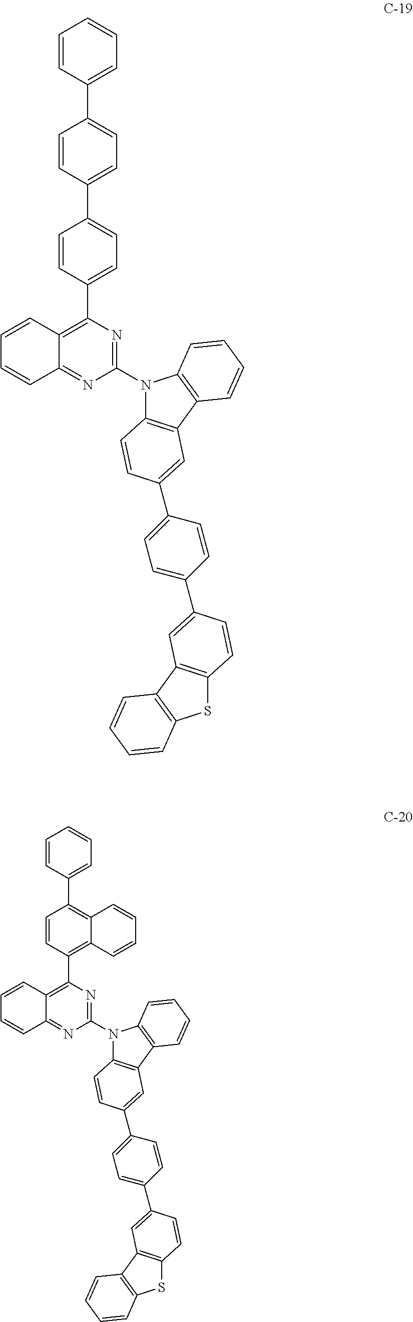 [SMILES: c1ccc(-c2ccc(-c3ccc(-c4nc(-n5c6ccccc6c6cc(-c7ccc(-c8ccc9sc%10ccccc%10c9c8)cc7)ccc65)nc5ccccc45)cc3)cc2)cc1 ; c1ccc(-c2ccc(-c3nc(-n4c5ccccc5c5cc(-c6ccc(-c7ccc8sc9ccccc9c8c7)cc6)ccc54)nc4ccccc34)c3ccccc23)cc1]

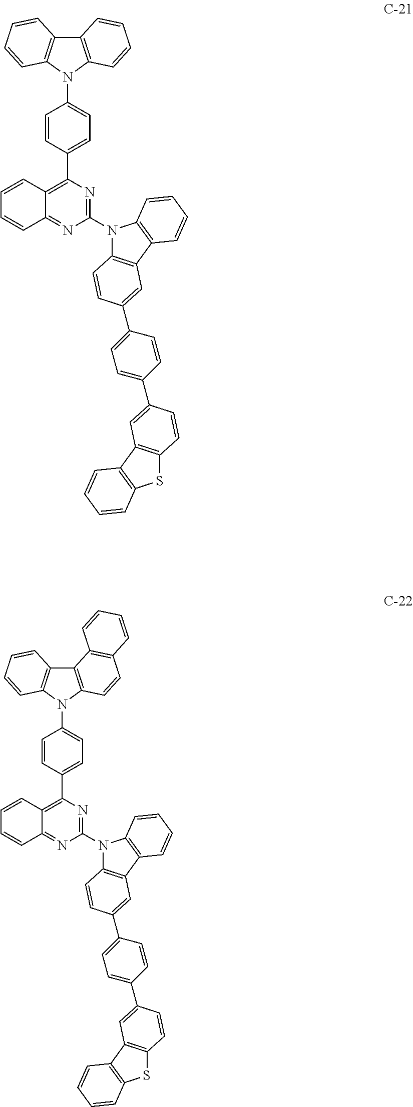 [SMILES: c1ccc2c(-c3ccc(-n4c5ccccc5c5ccccc54)cc3)nc(-n3c4ccccc4c4cc(-c5ccc(-c6ccc7sc8ccccc8c7c6)cc5)ccc43)nc2c1 ; c1ccc2c(c1)ccc1c2c2ccccc2n1-c1ccc(-c2nc(-n3c4ccccc4c4cc(-c5ccc(-c6ccc7sc8ccccc8c7c6)cc5)ccc43)nc3ccccc23)cc1]

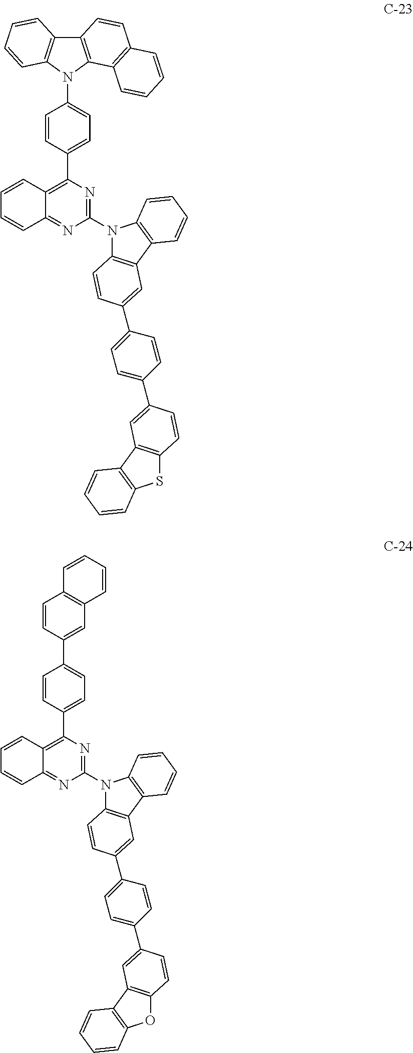 [SMILES: c1ccc2c(c1)ccc1c3ccccc3n(-c3ccc(-c4nc(-n5c6ccccc6c6cc(-c7ccc(-c8ccc9sc%10ccccc%10c9c8)cc7)ccc65)nc5ccccc45)cc3)c21 ; c1ccc2cc(-c3ccc(-c4nc(-n5c6ccccc6c6cc(-c7ccc(-c8ccc9oc%10ccccc%10c9c8)cc7)ccc65)nc5ccccc45)cc3)ccc2c1]

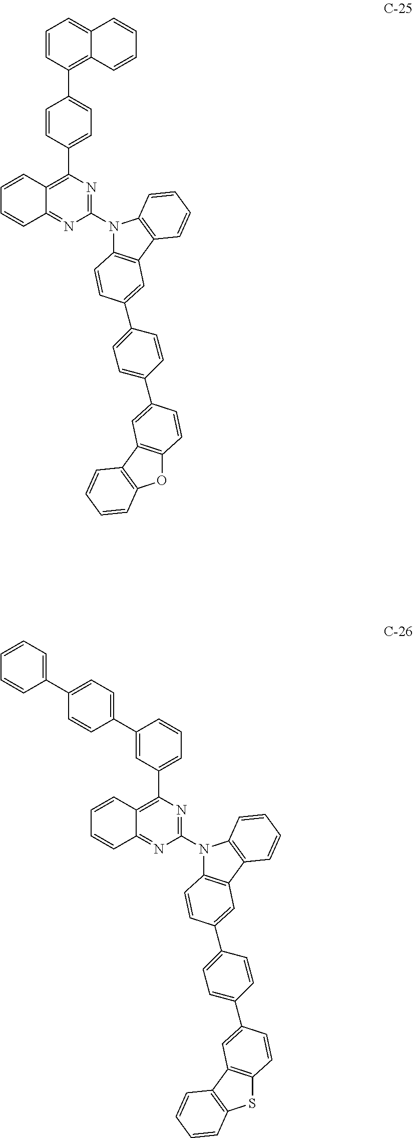 [SMILES: c1ccc2c(-c3ccc(-c4nc(-n5c6ccccc6c6cc(-c7ccc(-c8ccc9oc%10ccccc%10c9c8)cc7)ccc65)nc5ccccc45)cc3)cccc2c1 ; c1ccc(-c2ccc(-c3cccc(-c4nc(-n5c6ccccc6c6cc(-c7ccc(-c8ccc9sc%10ccccc%10c9c8)cc7)ccc65)nc5ccccc45)c3)cc2)cc1]

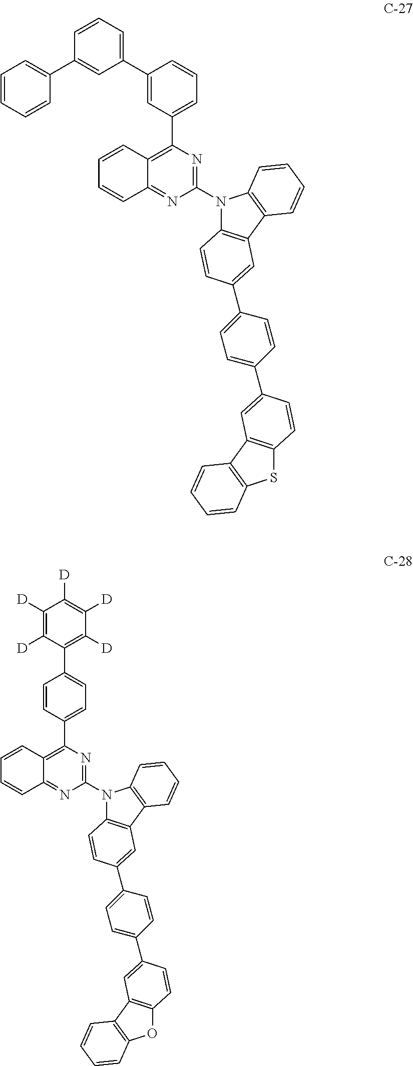 [SMILES: c1ccc(-c2cccc(-c3cccc(-c4nc(-n5c6ccccc6c6cc(-c7ccc(-c8ccc9sc%10ccccc%10c9c8)cc7)ccc65)nc5ccccc45)c3)c2)cc1 ; [2H]c1c([2H])c([2H])c(-c2ccc(-c3nc(-n4c5ccccc5c5cc(-c6ccc(-c7ccc8oc9ccccc9c8c7)cc6)ccc54)nc4ccccc34)cc2)c([2H])c1[2H]]

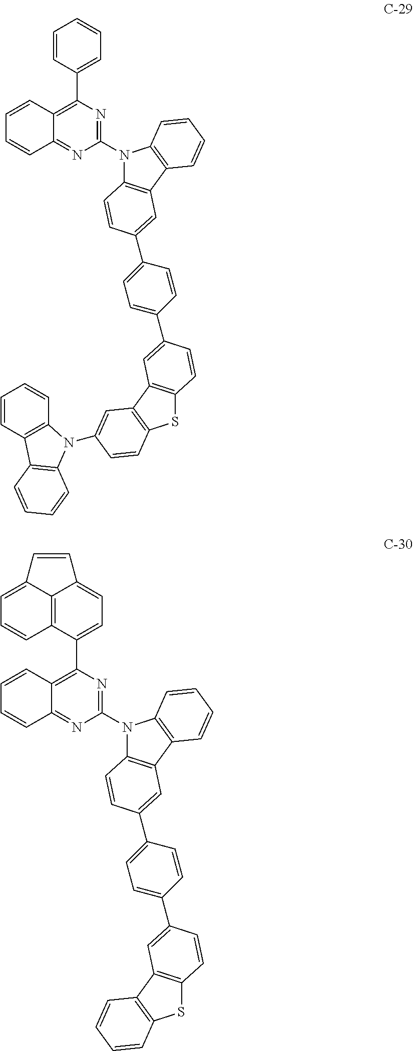 [SMILES: c1ccc(-c2nc(-n3c4ccccc4c4cc(-c5ccc(-c6ccc7sc8ccc(-n9c%10ccccc%10c%10ccccc%109)cc8c7c6)cc5)ccc43)nc3ccccc23)cc1 ; C1=Cc2ccc(-c3nc(-n4c5ccccc5c5cc(-c6ccc(-c7ccc8sc9ccccc9c8c7)cc6)ccc54)nc4ccccc34)c3cccc1c23]

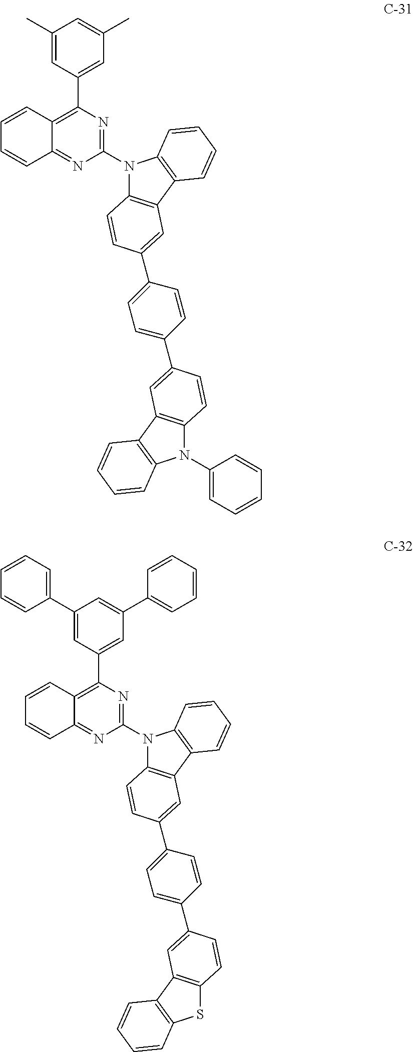 [SMILES: Cc1cc(C)cc(-c2nc(-n3c4ccccc4c4cc(-c5ccc(-c6ccc7c(c6)c6ccccc6n7-c6ccccc6)cc5)ccc43)nc3ccccc23)c1 ; c1ccc(-c2cc(-c3ccccc3)cc(-c3nc(-n4c5ccccc5c5cc(-c6ccc(-c7ccc8sc9ccccc9c8c7)cc6)ccc54)nc4ccccc34)c2)cc1]

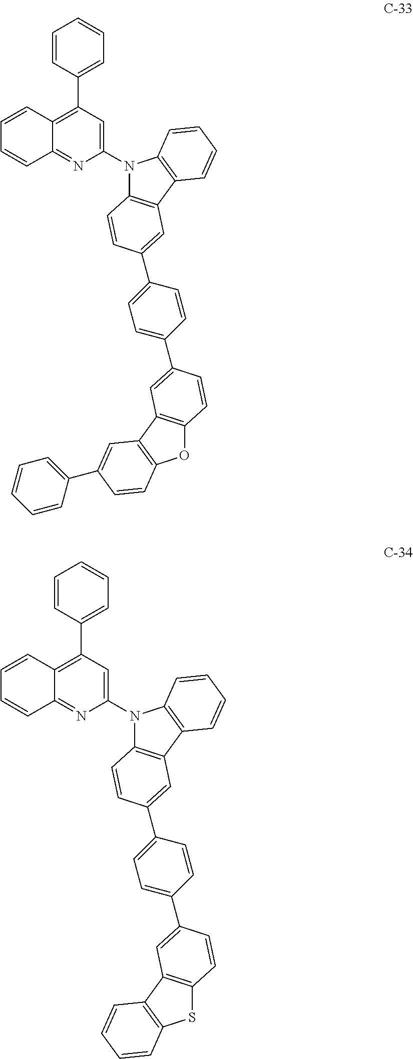 [SMILES: c1ccc(-c2ccc3oc4ccc(-c5ccc(-c6ccc7c(c6)c6ccccc6n7-c6cc(-c7ccccc7)c7ccccc7n6)cc5)cc4c3c2)cc1 ; c1ccc(-c2cc(-n3c4ccccc4c4cc(-c5ccc(-c6ccc7sc8ccccc8c7c6)cc5)ccc43)nc3ccccc23)cc1]

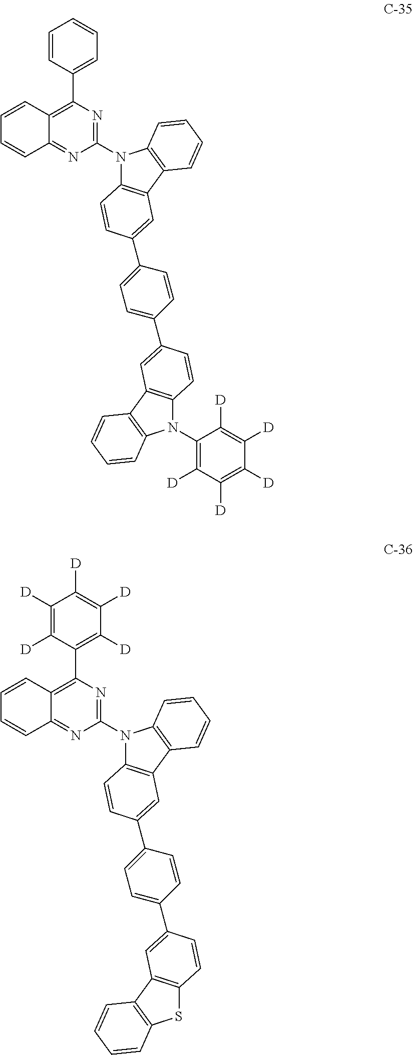 [SMILES: [2H]c1c([2H])c([2H])c(-n2c3ccccc3c3cc(-c4ccc(-c5ccc6c(c5)c5ccccc5n6-c5nc(-c6ccccc6)c6ccccc6n5)cc4)ccc32)c([2H])c1[2H] ; [2H]c1c([2H])c([2H])c(-c2nc(-n3c4ccccc4c4cc(-c5ccc(-c6ccc7sc8ccccc8c7c6)cc5)ccc43)nc3ccccc23)c([2H])c1[2H]]

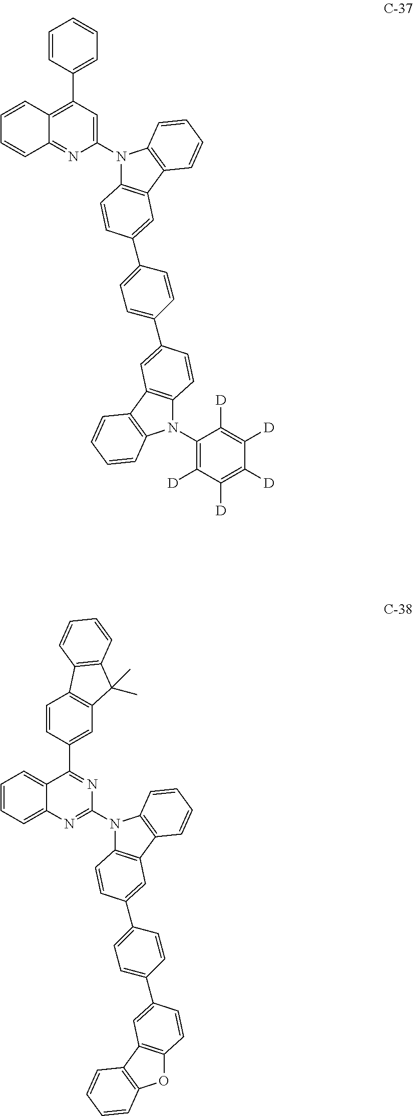 [SMILES: [2H]c1c([2H])c([2H])c(-n2c3ccccc3c3cc(-c4ccc(-c5ccc6c(c5)c5ccccc5n6-c5cc(-c6ccccc6)c6ccccc6n5)cc4)ccc32)c([2H])c1[2H] ; CC1(C)c2ccccc2-c2ccc(-c3nc(-n4c5ccccc5c5cc(-c6ccc(-c7ccc8oc9ccccc9c8c7)cc6)ccc54)nc4ccccc34)cc21]

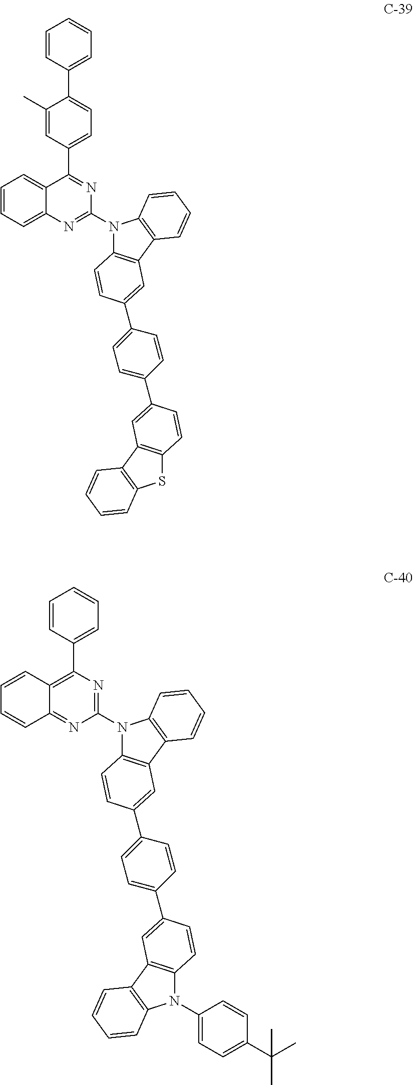 [SMILES: Cc1cc(-c2nc(-n3c4ccccc4c4cc(-c5ccc(-c6ccc7sc8ccccc8c7c6)cc5)ccc43)nc3ccccc23)ccc1-c1ccccc1 ; CC(C)(C)c1ccc(-n2c3ccccc3c3cc(-c4ccc(-c5ccc6c(c5)c5ccccc5n6-c5nc(-c6ccccc6)c6ccccc6n5)cc4)ccc32)cc1]

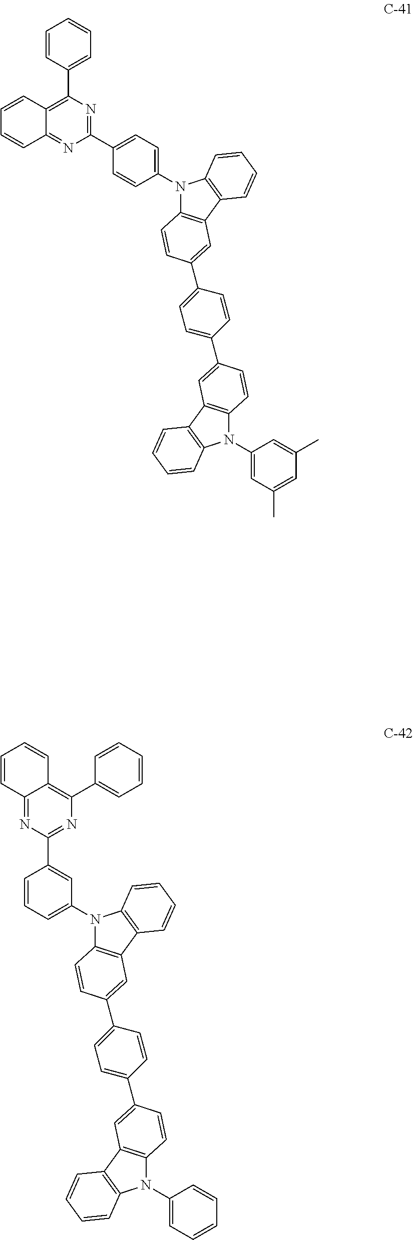 [SMILES: Cc1cc(C)cc(-n2c3ccccc3c3cc(-c4ccc(-c5ccc6c(c5)c5ccccc5n6-c5ccc(-c6nc(-c7ccccc7)c7ccccc7n6)cc5)cc4)ccc32)c1 ; c1ccc(-c2nc(-c3cccc(-n4c5ccccc5c5cc(-c6ccc(-c7ccc8c(c7)c7ccccc7n8-c7ccccc7)cc6)ccc54)c3)nc3ccccc23)cc1]

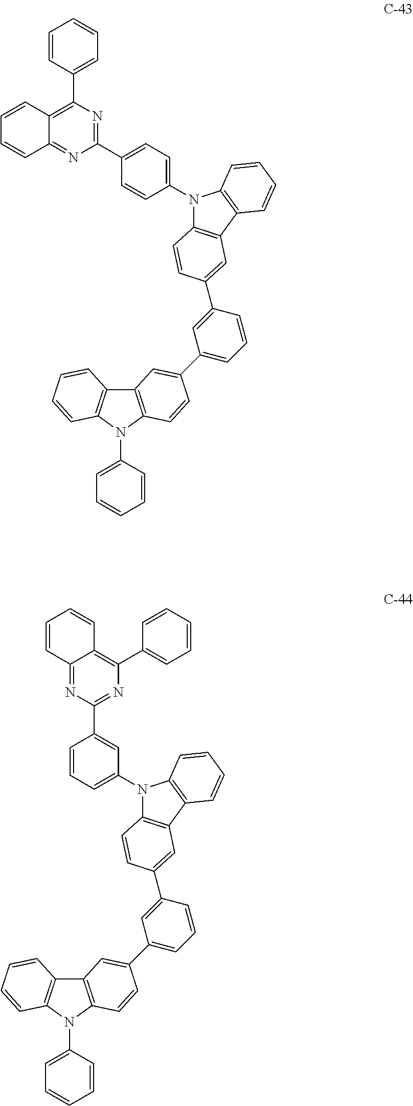 [SMILES: c1ccc(-c2nc(-c3ccc(-n4c5ccccc5c5cc(-c6cccc(-c7ccc8c(c7)c7ccccc7n8-c7ccccc7)c6)ccc54)cc3)nc3ccccc23)cc1 ; c1ccc(-c2nc(-c3cccc(-n4c5ccccc5c5cc(-c6cccc(-c7ccc8c(c7)c7ccccc7n8-c7ccccc7)c6)ccc54)c3)nc3ccccc23)cc1]

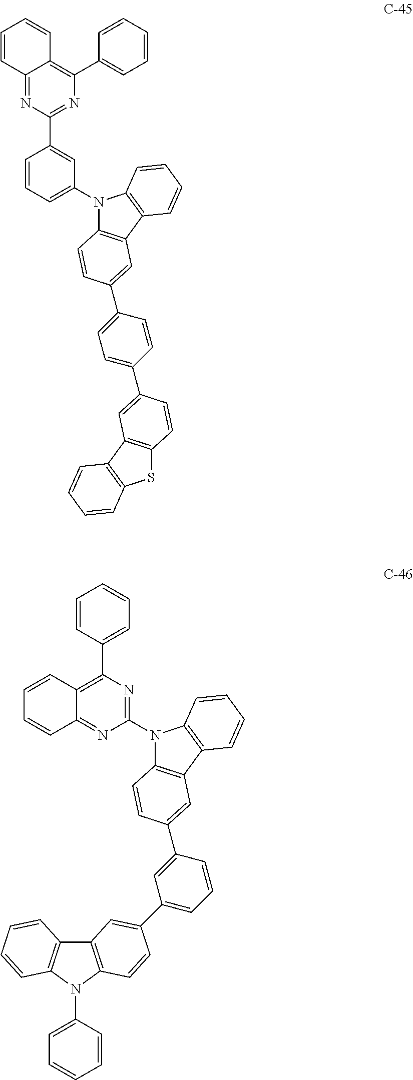 [SMILES: c1ccc(-c2nc(-c3cccc(-n4c5ccccc5c5cc(-c6ccc(-c7ccc8sc9ccccc9c8c7)cc6)ccc54)c3)nc3ccccc23)cc1 ; c1ccc(-c2nc(-n3c4ccccc4c4cc(-c5cccc(-c6ccc7c(c6)c6ccccc6n7-c6ccccc6)c5)ccc43)nc3ccccc23)cc1]

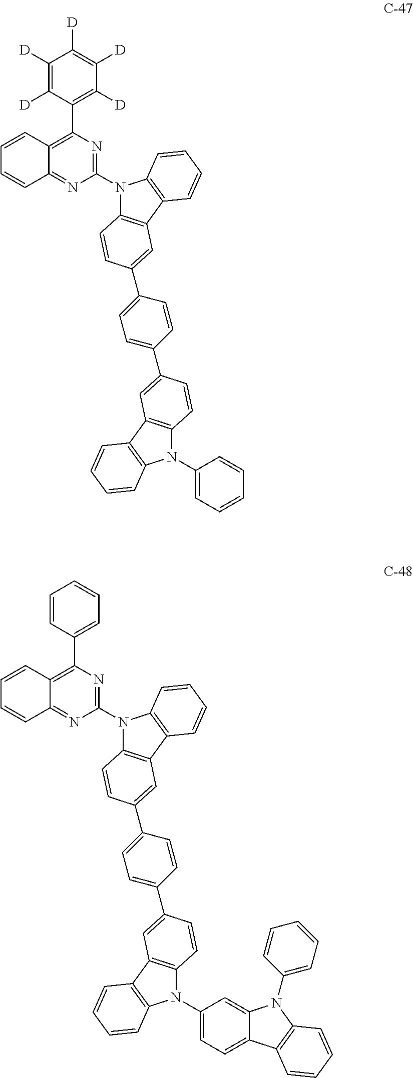 [SMILES: [2H]c1c([2H])c([2H])c(-c2nc(-n3c4ccccc4c4cc(-c5ccc(-c6ccc7c(c6)c6ccccc6n7-c6ccccc6)cc5)ccc43)nc3ccccc23)c([2H])c1[2H] ; c1ccc(-c2nc(-n3c4ccccc4c4cc(-c5ccc(-c6ccc7c(c6)c6ccccc6n7-c6ccc7c8ccccc8n(-c8ccccc8)c7c6)cc5)ccc43)nc3ccccc23)cc1]

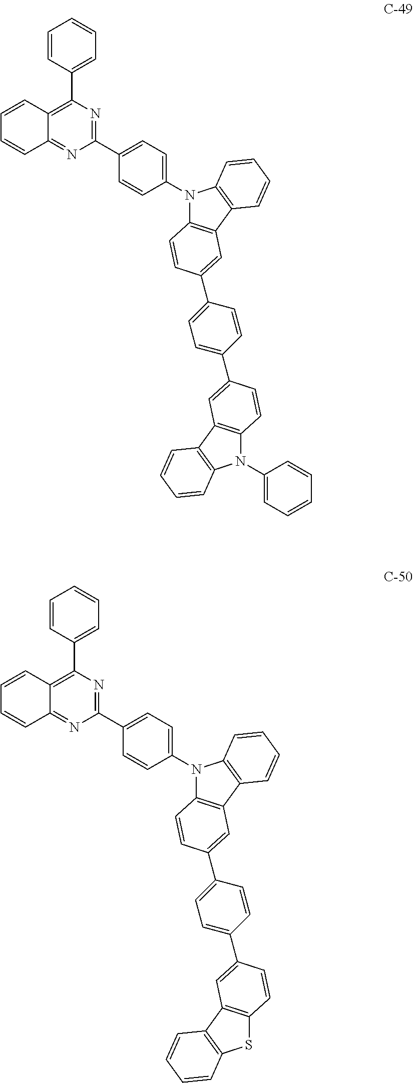 [SMILES: c1ccc(-c2nc(-c3ccc(-n4c5ccccc5c5cc(-c6ccc(-c7ccc8c(c7)c7ccccc7n8-c7ccccc7)cc6)ccc54)cc3)nc3ccccc23)cc1 ; c1ccc(-c2nc(-c3ccc(-n4c5ccccc5c5cc(-c6ccc(-c7ccc8sc9ccccc9c8c7)cc6)ccc54)cc3)nc3ccccc23)cc1]

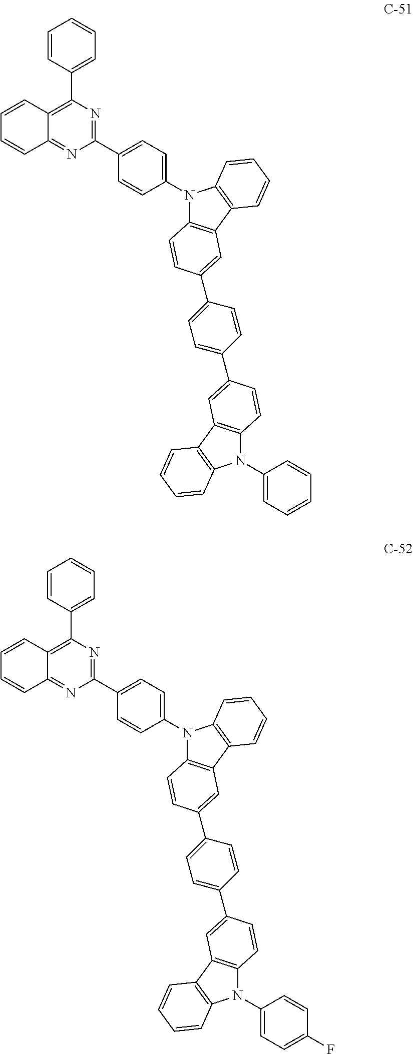 [SMILES: c1ccc(-c2nc(-c3ccc(-n4c5ccccc5c5cc(-c6ccc(-c7ccc8c(c7)c7ccccc7n8-c7ccccc7)cc6)ccc54)cc3)nc3ccccc23)cc1 ; Fc1ccc(-n2c3ccccc3c3cc(-c4ccc(-c5ccc6c(c5)c5ccccc5n6-c5ccc(-c6nc(-c7ccccc7)c7ccccc7n6)cc5)cc4)ccc32)cc1]

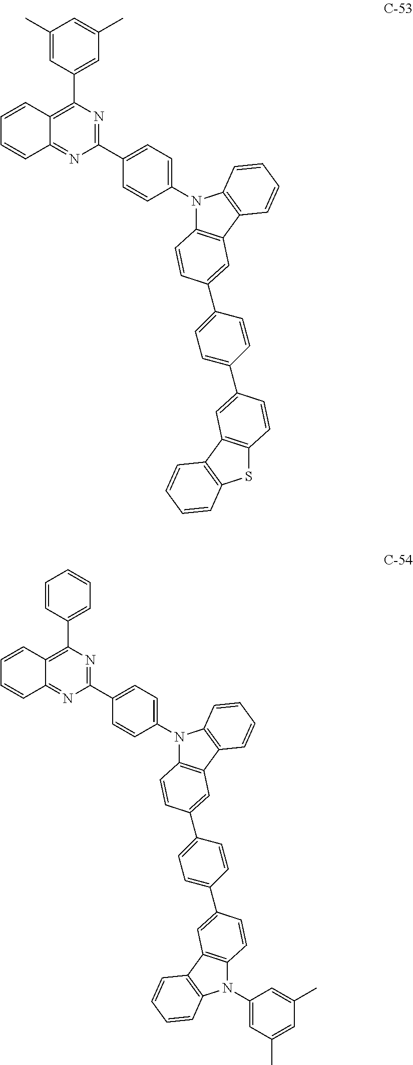 [SMILES: Cc1cc(C)cc(-c2nc(-c3ccc(-n4c5ccccc5c5cc(-c6ccc(-c7ccc8sc9ccccc9c8c7)cc6)ccc54)cc3)nc3ccccc23)c1 ; Cc1cc(C)cc(-n2c3ccccc3c3cc(-c4ccc(-c5ccc6c(c5)c5ccccc5n6-c5ccc(-c6nc(-c7ccccc7)c7ccccc7n6)cc5)cc4)ccc32)c1]

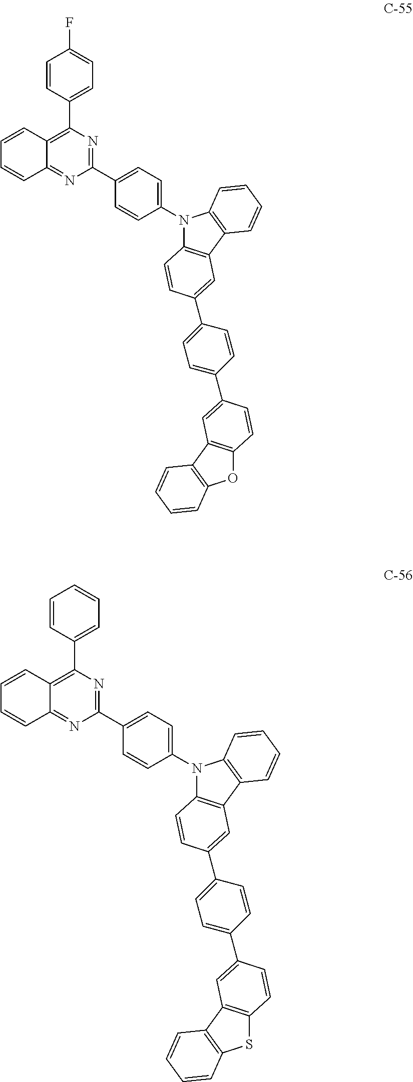 [SMILES: Fc1ccc(-c2nc(-c3ccc(-n4c5ccccc5c5cc(-c6ccc(-c7ccc8oc9ccccc9c8c7)cc6)ccc54)cc3)nc3ccccc23)cc1 ; c1ccc(-c2nc(-c3ccc(-n4c5ccccc5c5cc(-c6ccc(-c7ccc8sc9ccccc9c8c7)cc6)ccc54)cc3)nc3ccccc23)cc1]

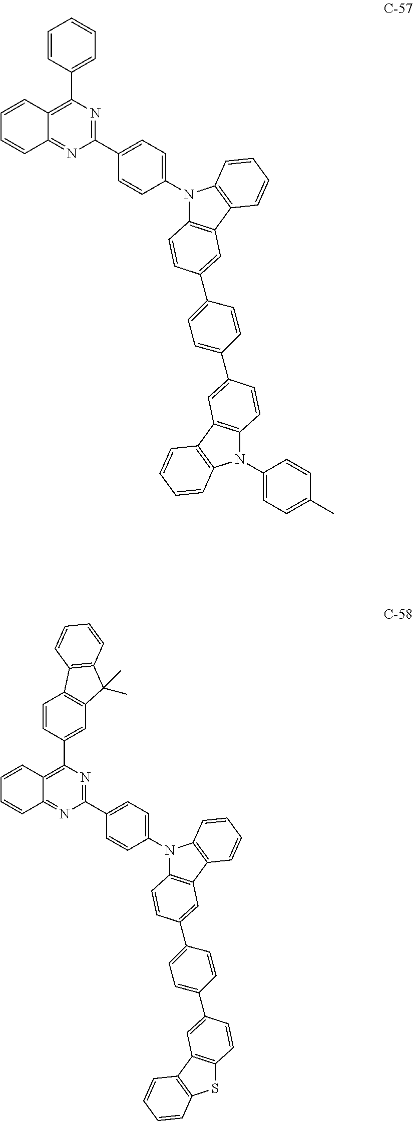 [SMILES: Cc1ccc(-n2c3ccccc3c3cc(-c4ccc(-c5ccc6c(c5)c5ccccc5n6-c5ccc(-c6nc(-c7ccccc7)c7ccccc7n6)cc5)cc4)ccc32)cc1 ; CC1(C)c2ccccc2-c2ccc(-c3nc(-c4ccc(-n5c6ccccc6c6cc(-c7ccc(-c8ccc9sc%10ccccc%10c9c8)cc7)ccc65)cc4)nc4ccccc34)cc21]

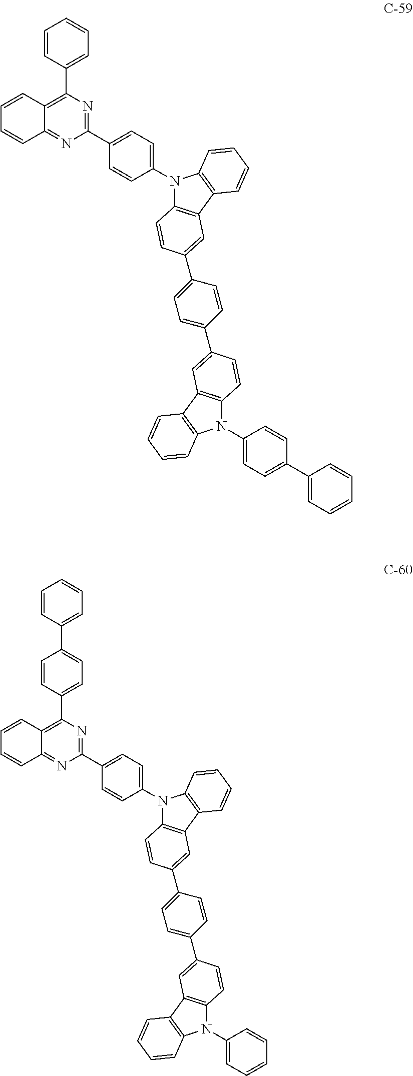 [SMILES: c1ccc(-c2ccc(-n3c4ccccc4c4cc(-c5ccc(-c6ccc7c(c6)c6ccccc6n7-c6ccc(-c7nc(-c8ccccc8)c8ccccc8n7)cc6)cc5)ccc43)cc2)cc1 ; c1ccc(-c2ccc(-c3nc(-c4ccc(-n5c6ccccc6c6cc(-c7ccc(-c8ccc9c(c8)c8ccccc8n9-c8ccccc8)cc7)ccc65)cc4)nc4ccccc34)cc2)cc1]

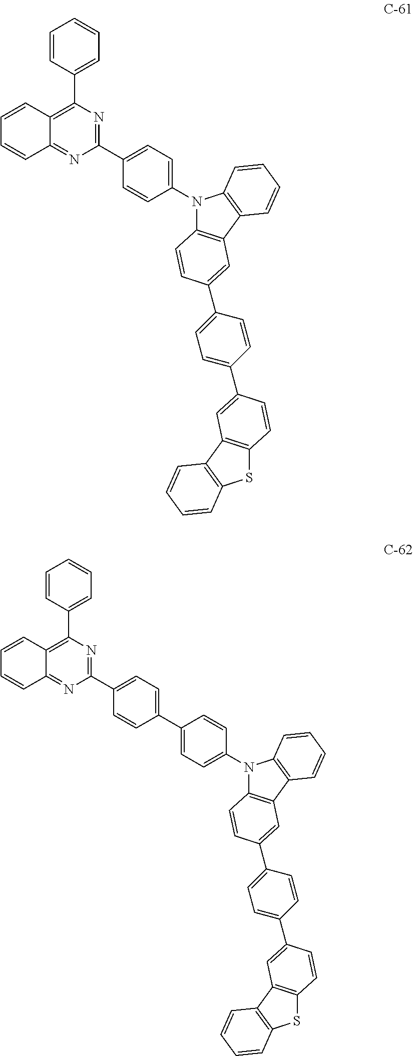 [SMILES: c1ccc(-c2nc(-c3ccc(-n4c5ccccc5c5cc(-c6ccc(-c7ccc8sc9ccccc9c8c7)cc6)ccc54)cc3)nc3ccccc23)cc1 ; c1ccc(-c2nc(-c3ccc(-c4ccc(-n5c6ccccc6c6cc(-c7ccc(-c8ccc9sc%10ccccc%10c9c8)cc7)ccc65)cc4)cc3)nc3ccccc23)cc1]

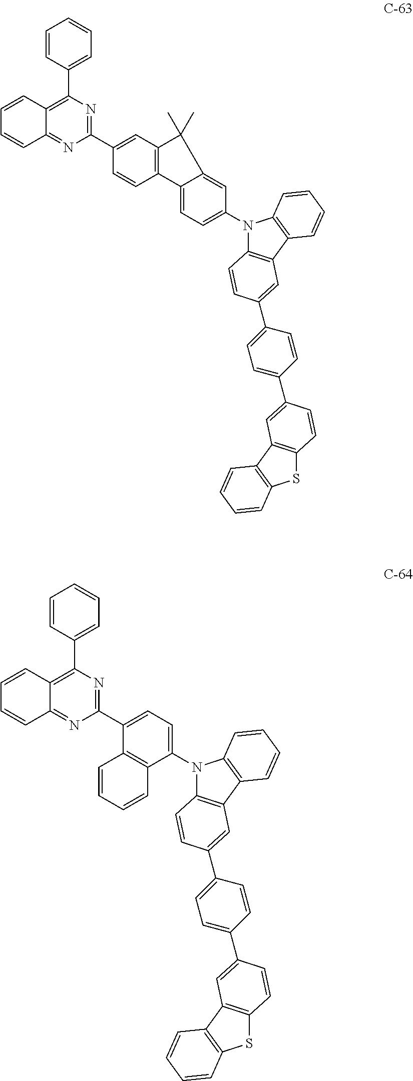 [SMILES: CC1(C)c2cc(-c3nc(-c4ccccc4)c4ccccc4n3)ccc2-c2ccc(-n3c4ccccc4c4cc(-c5ccc(-c6ccc7sc8ccccc8c7c6)cc5)ccc43)cc21 ; c1ccc(-c2nc(-c3ccc(-n4c5ccccc5c5cc(-c6ccc(-c7ccc8sc9ccccc9c8c7)cc6)ccc54)c4ccccc34)nc3ccccc23)cc1]

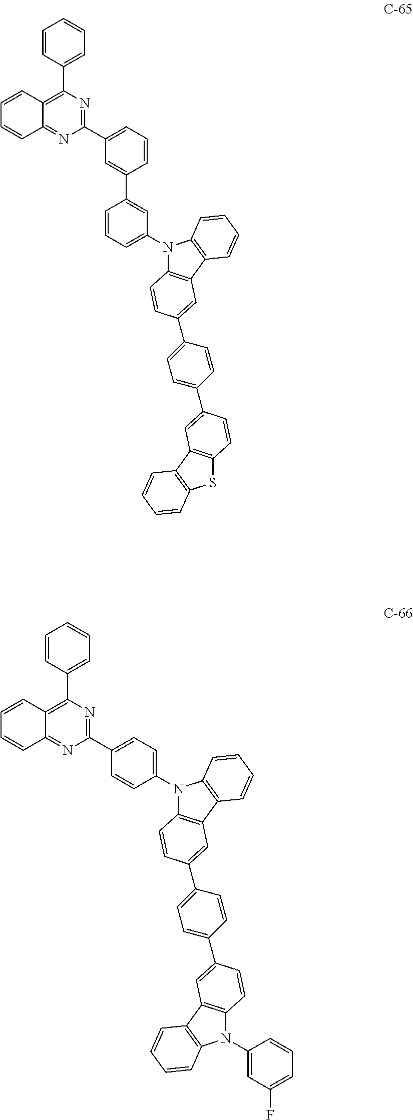 [SMILES: c1ccc(-c2nc(-c3cccc(-c4cccc(-n5c6ccccc6c6cc(-c7ccc(-c8ccc9sc%10ccccc%10c9c8)cc7)ccc65)c4)c3)nc3ccccc23)cc1 ; Fc1cccc(-n2c3ccccc3c3cc(-c4ccc(-c5ccc6c(c5)c5ccccc5n6-c5ccc(-c6nc(-c7ccccc7)c7ccccc7n6)cc5)cc4)ccc32)c1]

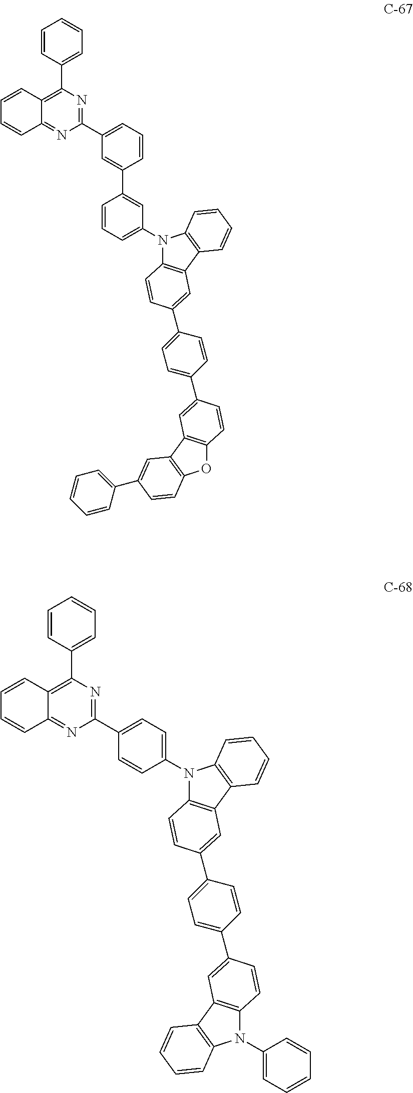 [SMILES: c1ccc(-c2ccc3oc4ccc(-c5ccc(-c6ccc7c(c6)c6ccccc6n7-c6cccc(-c7cccc(-c8nc(-c9ccccc9)c9ccccc9n8)c7)c6)cc5)cc4c3c2)cc1 ; c1ccc(-c2nc(-c3ccc(-n4c5ccccc5c5cc(-c6ccc(-c7ccc8c(c7)c7ccccc7n8-c7ccccc7)cc6)ccc54)cc3)nc3ccccc23)cc1]

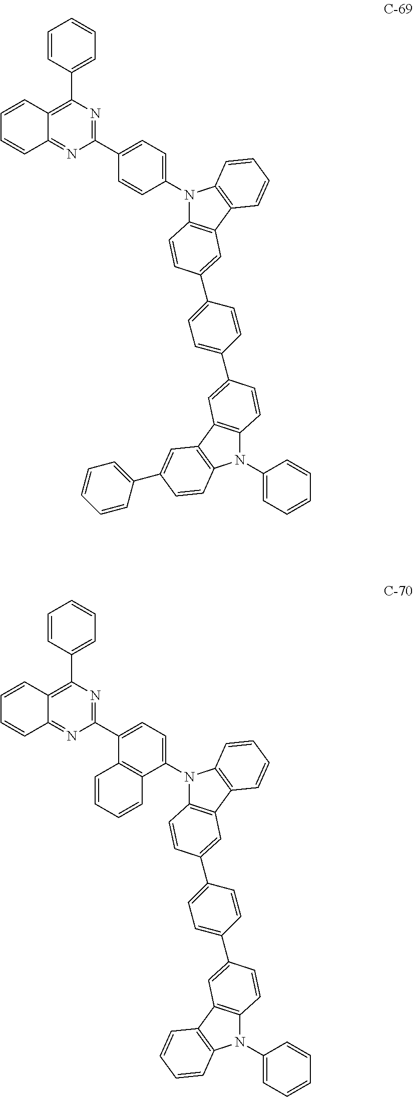 [SMILES: c1ccc(-c2ccc3c(c2)c2cc(-c4ccc(-c5ccc6c(c5)c5ccccc5n6-c5ccc(-c6nc(-c7ccccc7)c7ccccc7n6)cc5)cc4)ccc2n3-c2ccccc2)cc1 ; c1ccc(-c2nc(-c3ccc(-n4c5ccccc5c5cc(-c6ccc(-c7ccc8c(c7)c7ccccc7n8-c7ccccc7)cc6)ccc54)c4ccccc34)nc3ccccc23)cc1]

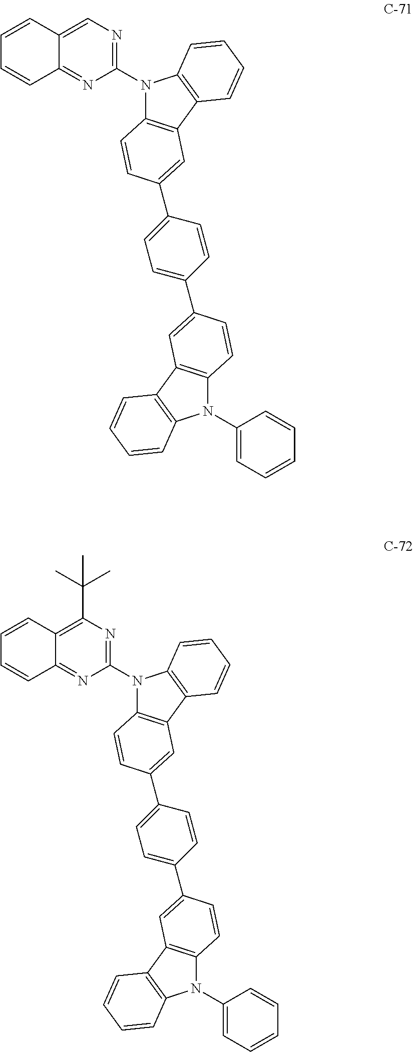 [SMILES: c1ccc(-n2c3ccccc3c3cc(-c4ccc(-c5ccc6c(c5)c5ccccc5n6-c5ncc6ccccc6n5)cc4)ccc32)cc1 ; CC(C)(C)c1nc(-n2c3ccccc3c3cc(-c4ccc(-c5ccc6c(c5)c5ccccc5n6-c5ccccc5)cc4)ccc32)nc2ccccc12]

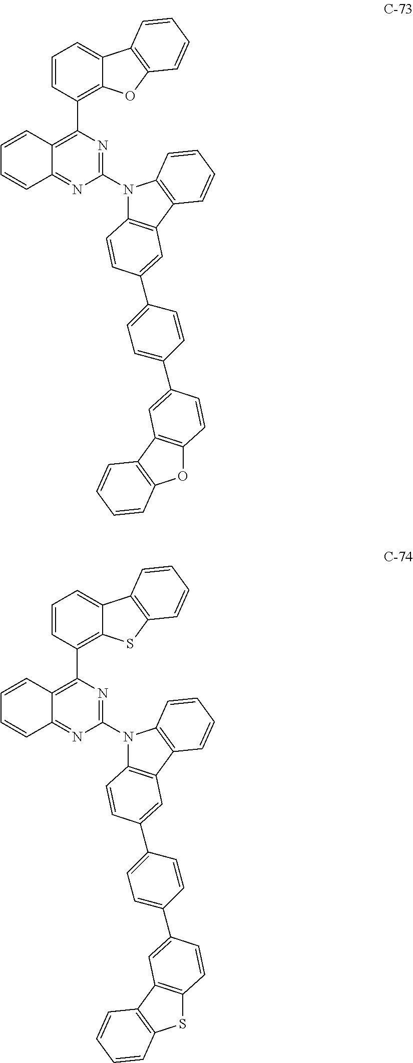 [SMILES: c1ccc2c(-c3cccc4c3oc3ccccc34)nc(-n3c4ccccc4c4cc(-c5ccc(-c6ccc7oc8ccccc8c7c6)cc5)ccc43)nc2c1 ; c1ccc2c(-c3cccc4c3sc3ccccc34)nc(-n3c4ccccc4c4cc(-c5ccc(-c6ccc7sc8ccccc8c7c6)cc5)ccc43)nc2c1]

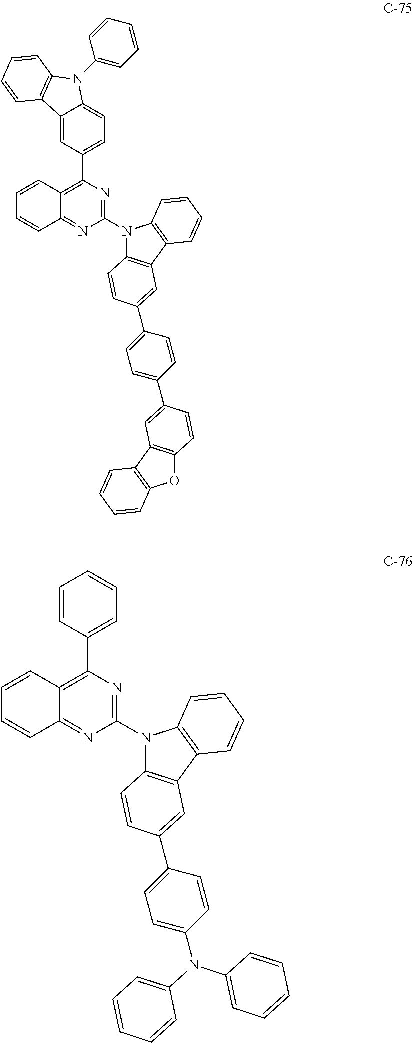 [SMILES: c1ccc(-n2c3ccccc3c3cc(-c4nc(-n5c6ccccc6c6cc(-c7ccc(-c8ccc9oc%10ccccc%10c9c8)cc7)ccc65)nc5ccccc45)ccc32)cc1 ; c1ccc(-c2nc(-n3c4ccccc4c4cc(-c5ccc(N(c6ccccc6)c6ccccc6)cc5)ccc43)nc3ccccc23)cc1]

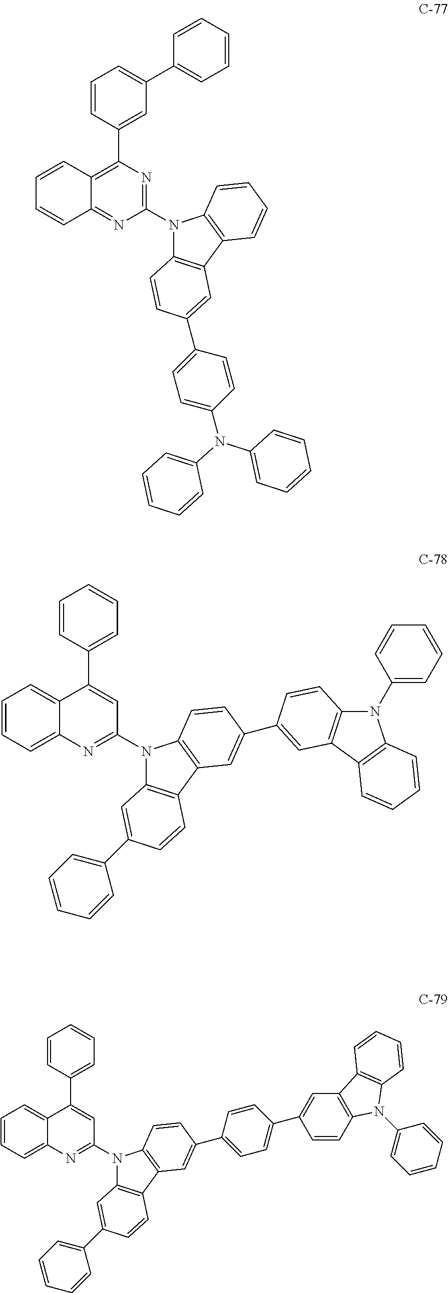 [SMILES: c1ccc(-c2cccc(-c3nc(-n4c5ccccc5c5cc(-c6ccc(N(c7ccccc7)c7ccccc7)cc6)ccc54)nc4ccccc34)c2)cc1 ; c1ccc(-c2ccc3c4cc(-c5ccc6c(c5)c5ccccc5n6-c5ccccc5)ccc4n(-c4cc(-c5ccccc5)c5ccccc5n4)c3c2)cc1 ; c1ccc(-c2ccc3c4cc(-c5ccc(-c6ccc7c(c6)c6ccccc6n7-c6ccccc6)cc5)ccc4n(-c4cc(-c5ccccc5)c5ccccc5n4)c3c2)cc1]

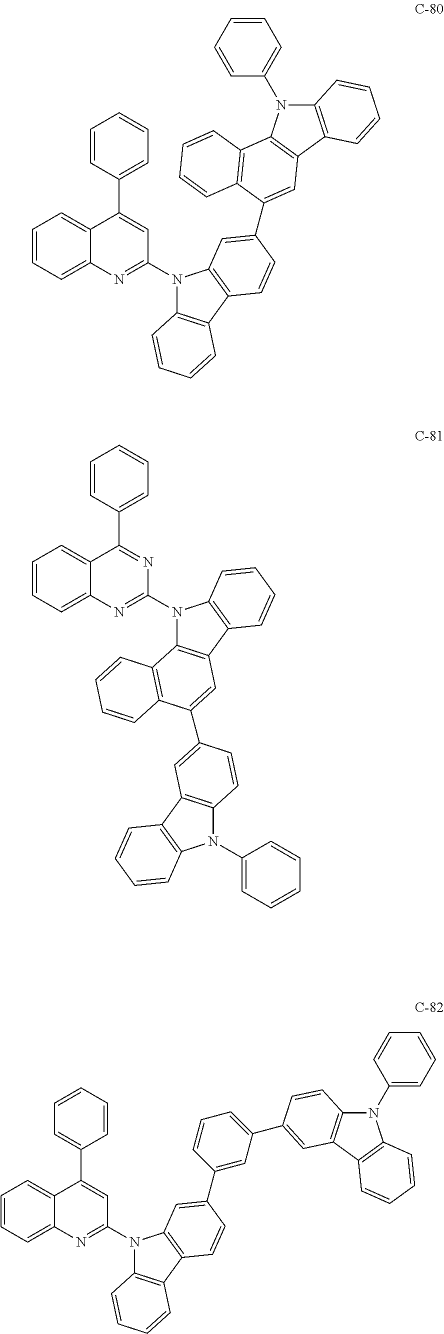 [SMILES: c1ccc(-c2cc(-n3c4ccccc4c4ccc(-c5cc6c7ccccc7n(-c7ccccc7)c6c6ccccc56)cc43)nc3ccccc23)cc1 ; c1ccc(-c2nc(-n3c4ccccc4c4cc(-c5ccc6c(c5)c5ccccc5n6-c5ccccc5)c5ccccc5c43)nc3ccccc23)cc1 ; c1ccc(-c2cc(-n3c4ccccc4c4ccc(-c5cccc(-c6ccc7c(c6)c6ccccc6n7-c6ccccc6)c5)cc43)nc3ccccc23)cc1]

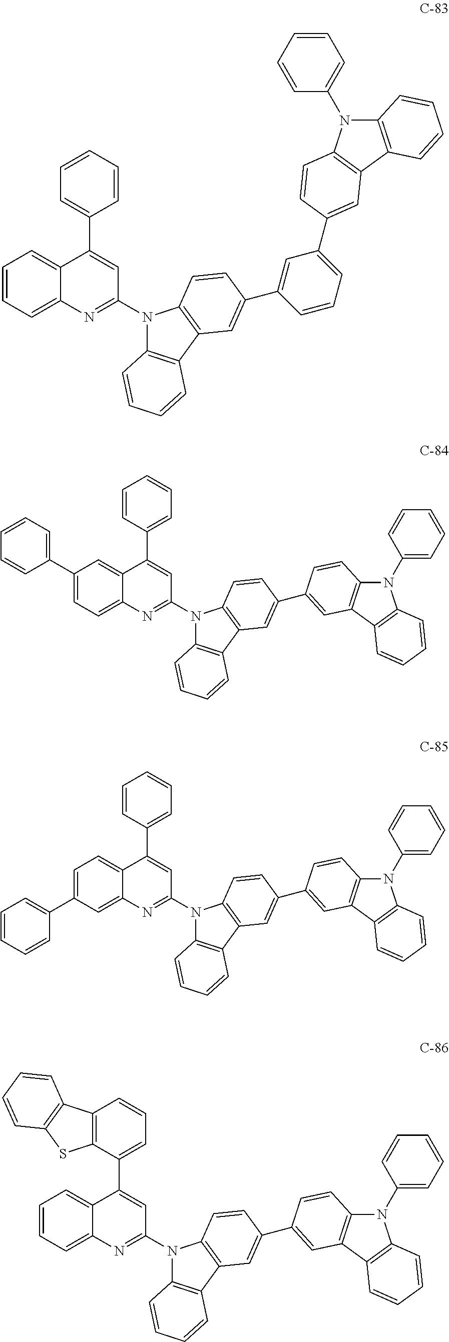 [SMILES: c1ccc(-c2cc(-n3c4ccccc4c4cc(-c5cccc(-c6ccc7c(c6)c6ccccc6n7-c6ccccc6)c5)ccc43)nc3ccccc23)cc1 ; c1ccc(-c2ccc3nc(-n4c5ccccc5c5cc(-c6ccc7c(c6)c6ccccc6n7-c6ccccc6)ccc54)cc(-c4ccccc4)c3c2)cc1 ; c1ccc(-c2ccc3c(-c4ccccc4)cc(-n4c5ccccc5c5cc(-c6ccc7c(c6)c6ccccc6n7-c6ccccc6)ccc54)nc3c2)cc1 ; c1ccc(-n2c3ccccc3c3cc(-c4ccc5c(c4)c4ccccc4n5-c4cc(-c5cccc6c5sc5ccccc56)c5ccccc5n4)ccc32)cc1]

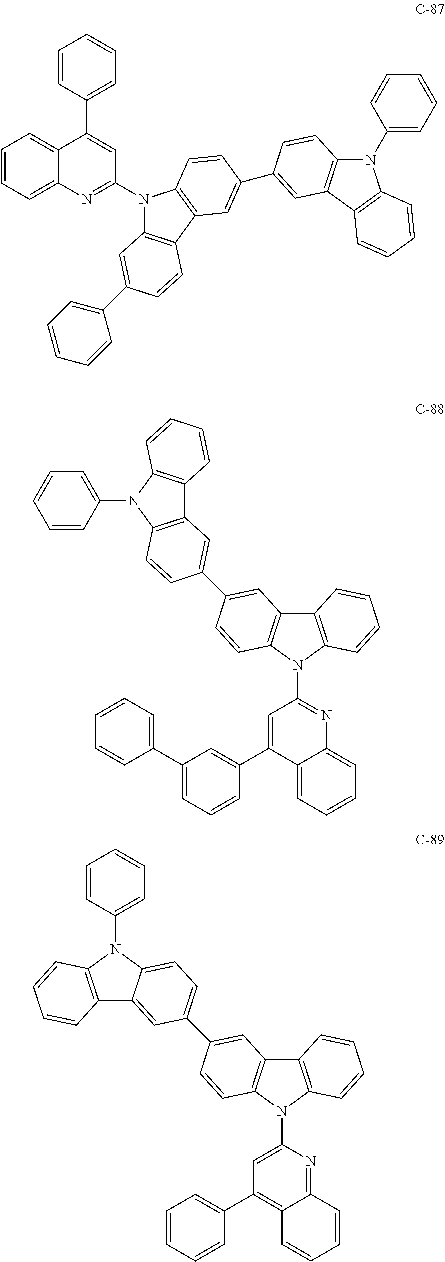 [SMILES: c1ccc(-c2ccc3c4cc(-c5ccc6c(c5)c5ccccc5n6-c5ccccc5)ccc4n(-c4cc(-c5ccccc5)c5ccccc5n4)c3c2)cc1 ; c1ccc(-c2cccc(-c3cc(-n4c5ccccc5c5cc(-c6ccc7c(c6)c6ccccc6n7-c6ccccc6)ccc54)nc4ccccc34)c2)cc1 ; c1ccc(-c2cc(-n3c4ccccc4c4cc(-c5ccc6c(c5)c5ccccc5n6-c5ccccc5)ccc43)nc3ccccc23)cc1]

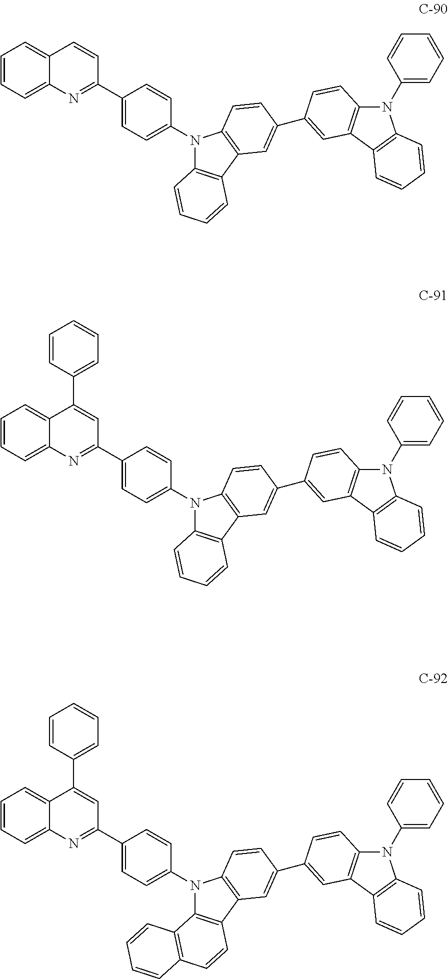 [SMILES: c1ccc(-n2c3ccccc3c3cc(-c4ccc5c(c4)c4ccccc4n5-c4ccc(-c5ccc6ccccc6n5)cc4)ccc32)cc1 ; c1ccc(-c2cc(-c3ccc(-n4c5ccccc5c5cc(-c6ccc7c(c6)c6ccccc6n7-c6ccccc6)ccc54)cc3)nc3ccccc23)cc1 ; c1ccc(-c2cc(-c3ccc(-n4c5ccc(-c6ccc7c(c6)c6ccccc6n7-c6ccccc6)cc5c5ccc6ccccc6c54)cc3)nc3ccccc23)cc1]

The organic electroluminescent compounds according to the present invention can be prepared according to the following reaction scheme 1.

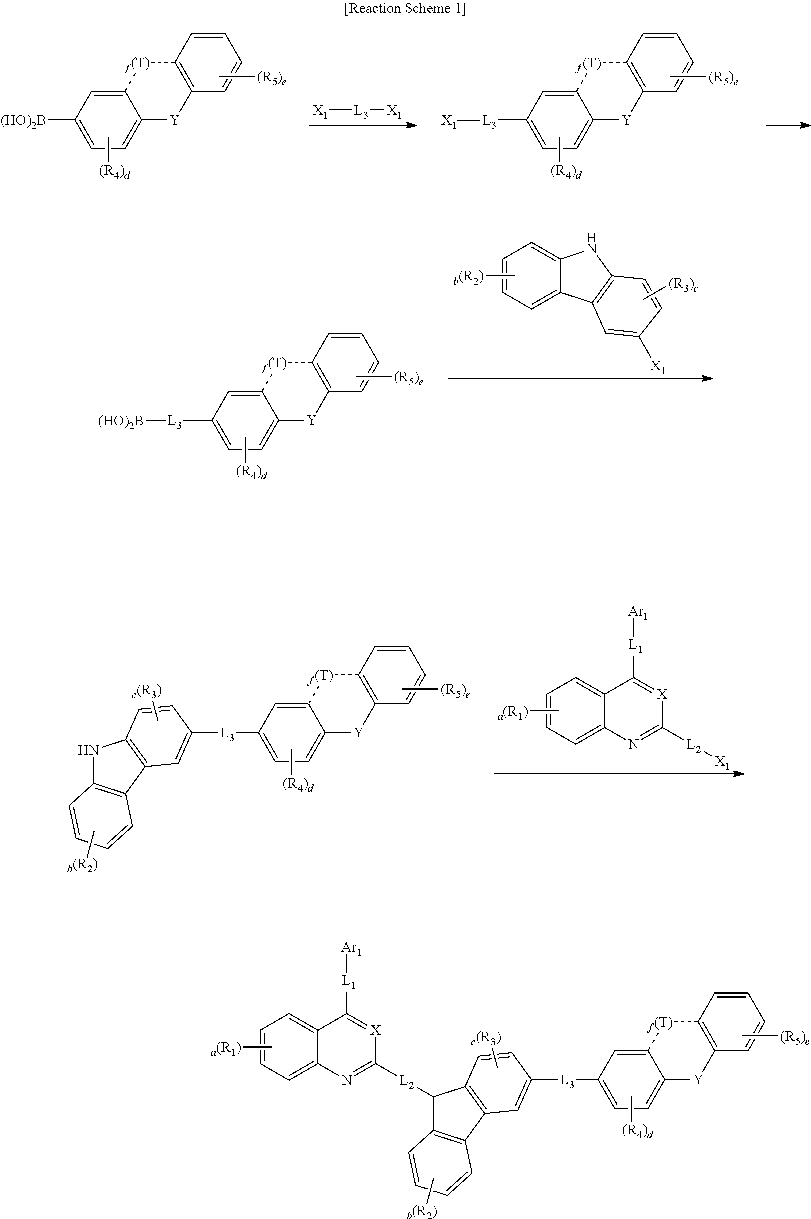

wherein Ar₁, R₁ to R₅, Y, X, T, L₁, L₂, L₃, a, b, c, d, e and f are as defined in formula 1, and X₁ represents a halogen.

In addition, the present invention provides an organic electroluminescent material comprising the organic electroluminescent compound of formula 1, and an organic electroluminescent device comprising the material. The material can be comprised of the organic electroluminescent compound according to the present invention alone, or can further include conventional materials generally used in organic electroluminescent materials. The organic electroluminescent device according to the present invention comprises a first electrode, a second electrode, and at least one organic layer between said first and second electrodes. The organic layer comprises at least one compound of formula 1. Further, the organic layer comprises a light-emitting layer in which the organic electroluminescent compound of formula 1 may be used as a host material.

When the organic electroluminescent compounds of formula 1 is used as a host in the light-emitting layer, the light-emitting layer may comprise at least one phosphorescent dopant. The phosphorescent dopant for an organic electroluminescent device of the present invention is not specifically limited, but is preferably selected from compounds represented by the following formula 2:

M¹L¹⁰¹L¹⁰²L¹⁰³ (2)

wherein M¹ is selected from the group consisting of Ir, Pt, Pd and Os;

ligand L¹⁰¹, L¹⁰² and L¹⁰³ are each independently selected from the following structures:

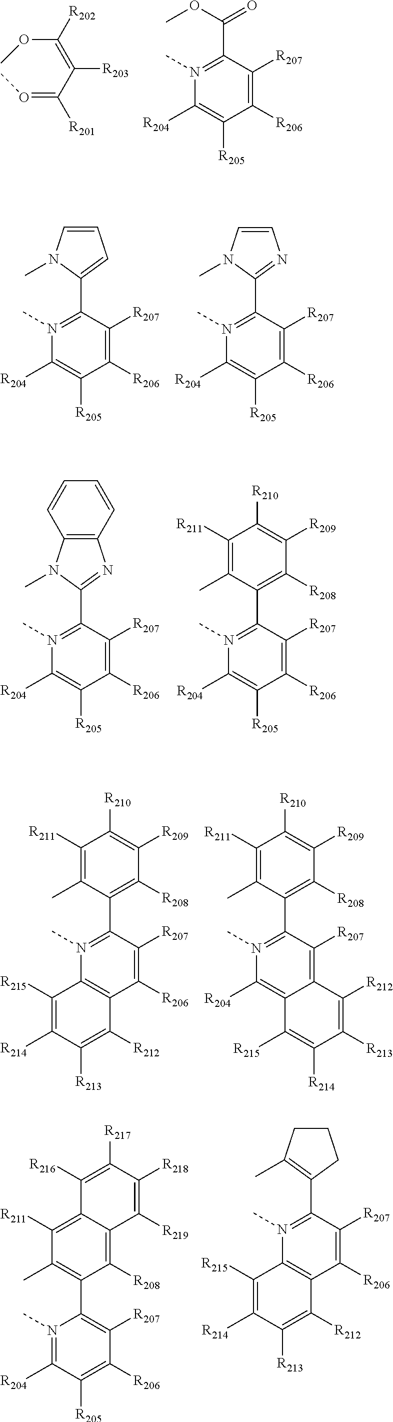

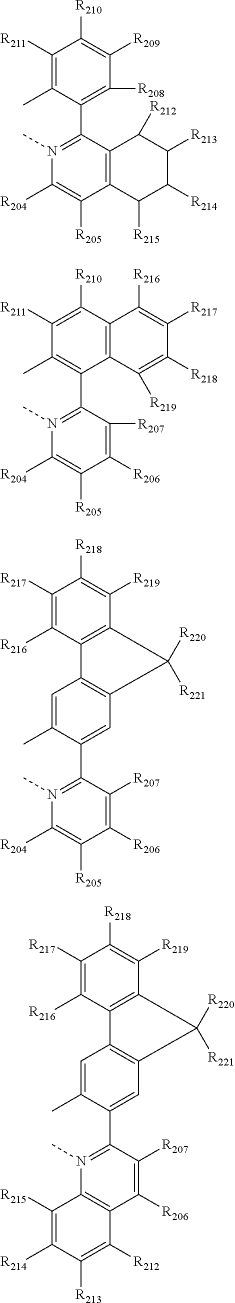

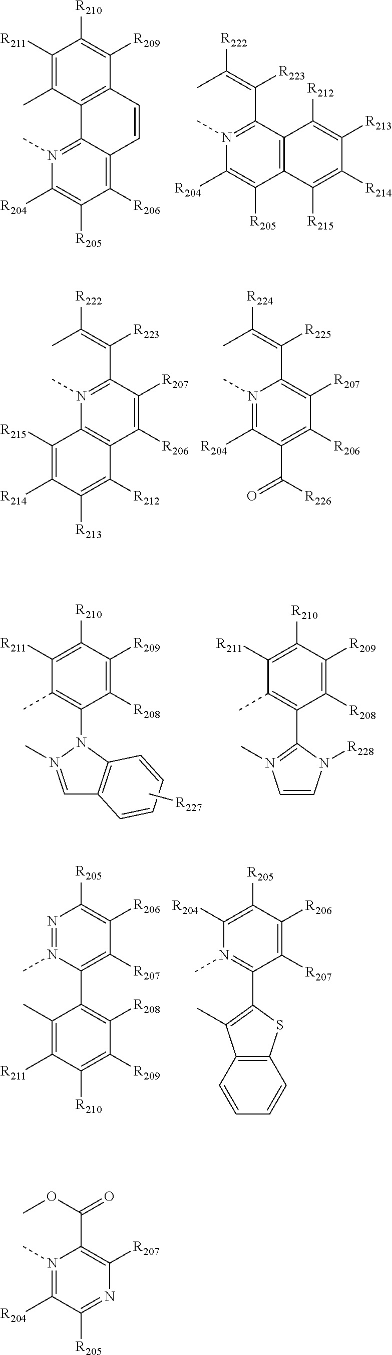

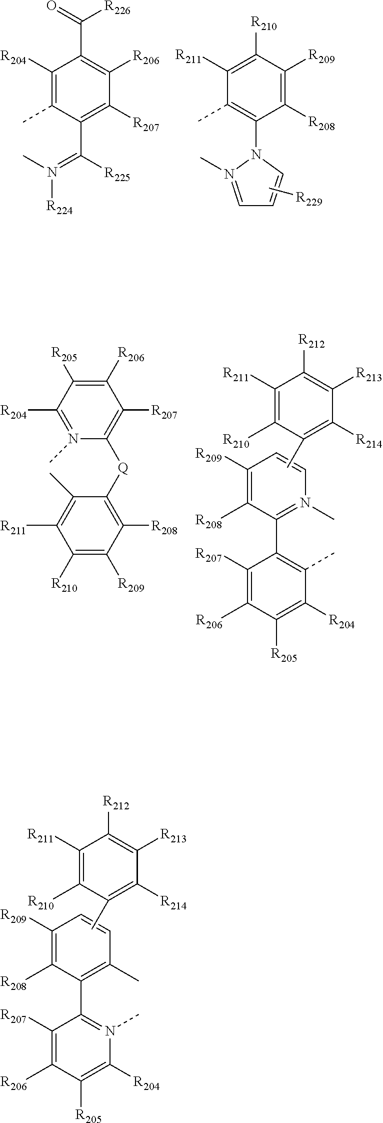

R₂₀₁ to R₂₀₃ each independently represent hydrogen, deuterium, a (C1-C30)alkyl group which is unsubstituted or substituted with a halogen, a substituted or unsubstituted (C3-C30)cycloalkyl group, a (C6-C30)aryl group which is unsubstituted or substituted with a (C1-C30)alkyl group, or a halogen;

R₂₀₄ to R₂₁₉ each independently represent hydrogen, deuterium, a substituted or unsubstituted (C1-C30)alkyl group, a substituted or unsubstituted (C1-C30)alkoxy group, a substituted or unsubstituted (C3-C30)cycloalkyl group, a substituted or unsubstituted (C2-C30)alkenyl group, a substituted or unsubstituted (C6-C30)aryl group, a substituted or unsubstituted mono- or di-(C1-C30)alkylamino group, a substituted or unsubstituted mono- or di-(C6-C30)arylamino group, SF₅, a substituted or unsubstituted tri(C1-C30)alkylsilyl group, a substituted or unsubstituted di(C1-C30)alkyl(C6-C30)arylsilyl group, a substituted or unsubstituted tri(C6-C30)arylsilyl group, a cyano group or a halogen;

R₂₂₀ to R₂₂₃ each independently represent hydrogen, deuterium, a (C1-C30)alkyl group which is unsubstituted or substituted with a halogen, or a (C6-C30)aryl group unsubstituted or substituted with a (C1-C30)alkyl group;

R₂₂₄ and R₂₂₅ each independently represent hydrogen, deuterium, a substituted or unsubstituted (C1-C30)alkyl group, a substituted or unsubstituted (C6-C30)aryl group, or a halogen, or R₂₂₄ and R₂₂₅ are linked to each other to form a mono- or polycyclic, (C5-C30)alicyclic or aromatic ring;

R₂₂₆ represents a substituted or unsubstituted (C1-C30)alkyl group, a substituted or unsubstituted (C6-C30)aryl group, a substituted or unsubstituted 3- or 30-membered heteroaryl group or a halogen;

R₂₂₇ to R₂₂₉ each independently represent hydrogen, deuterium, a substituted or unsubstituted (C1-C30)alkyl group, a substituted or unsubstituted (C6-C30)aryl group or a halogen;

Q represents

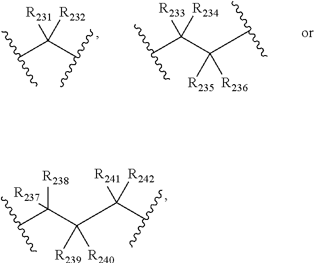

wherein R₂₃₁ to R₂₄₂ each independently represent hydrogen, deuterium, a (C1-C30)alkyl group which is unsubstituted or substituted with a halogen, a (C1-C30)alkoxy group, a halogen, a substituted or unsubstituted (C6-C30)aryl group, a cyano group, or a substituted or unsubstituted (C5-C30)cycloalkyl group, or each of R₂₃₁ to R₂₄₂ may be linked to an adjacent substituent(s) to form a spiro ring or a fused ring or may be linked to R₂₀₇ or R₂₀₈ to form a saturated or unsaturated fused ring.

The dopants of formula 2 include the following compounds, but are not limited thereto:

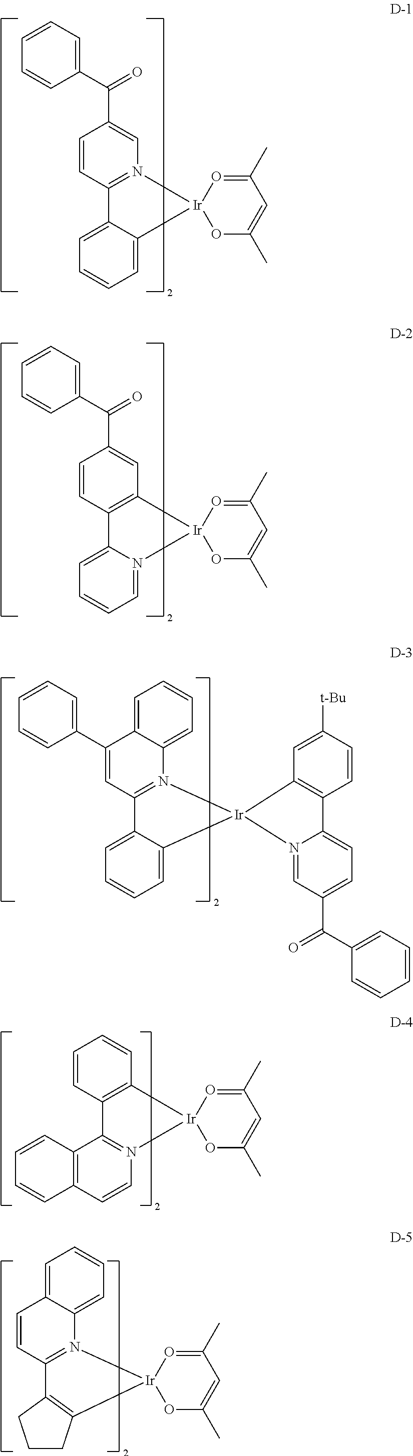

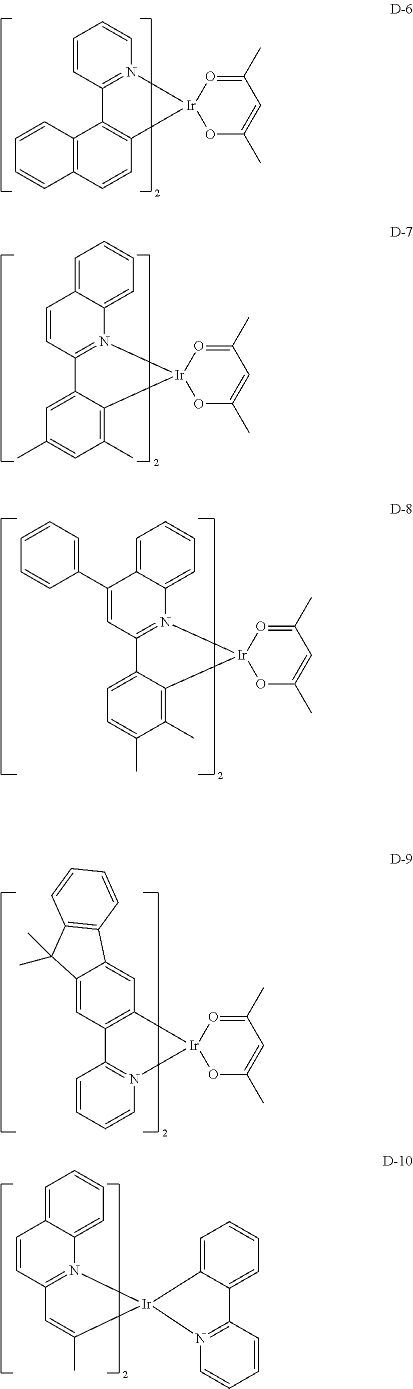

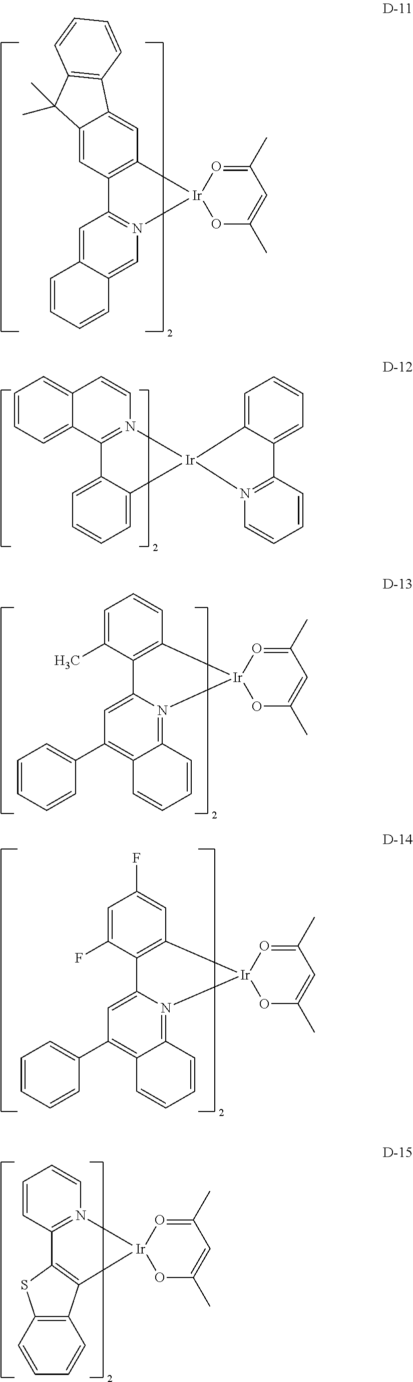

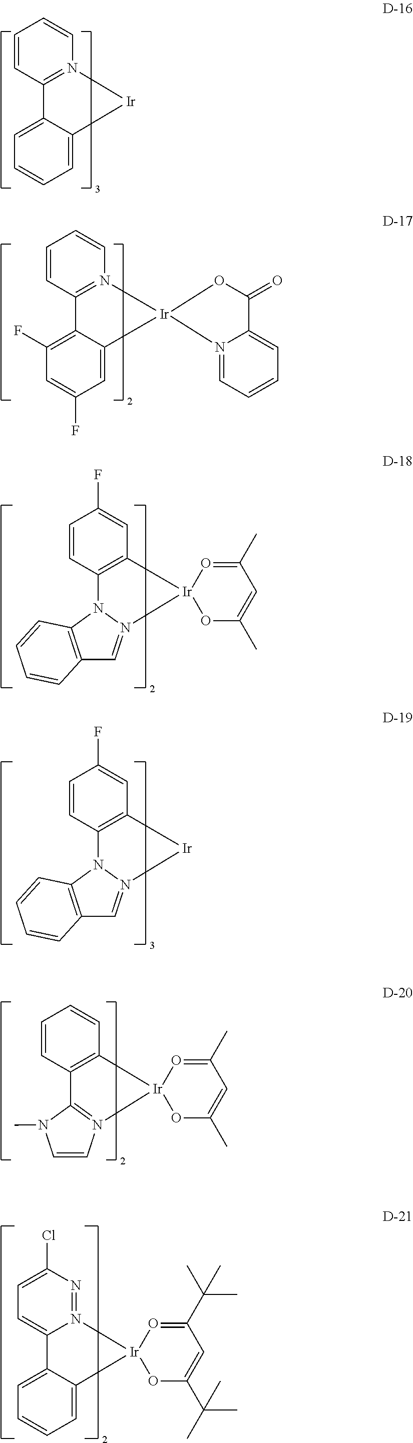

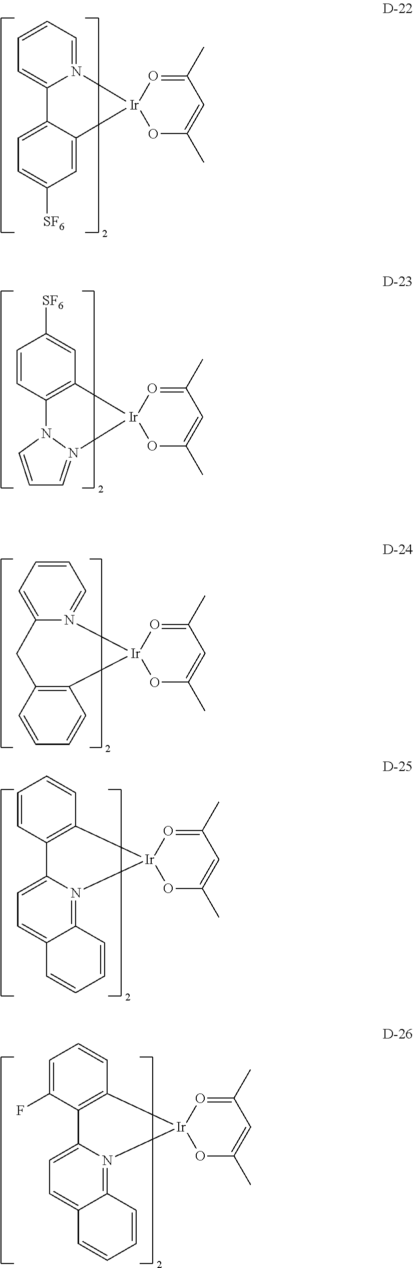

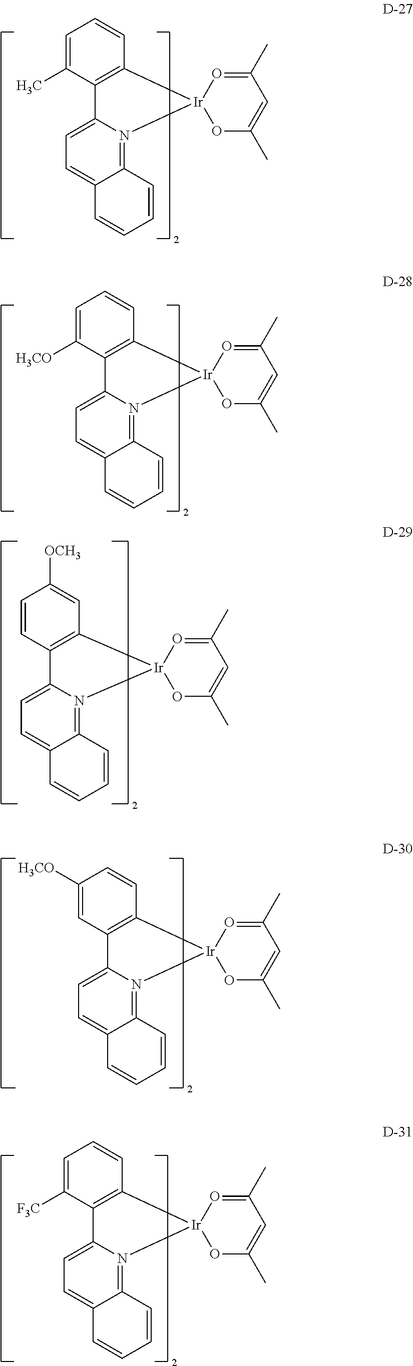

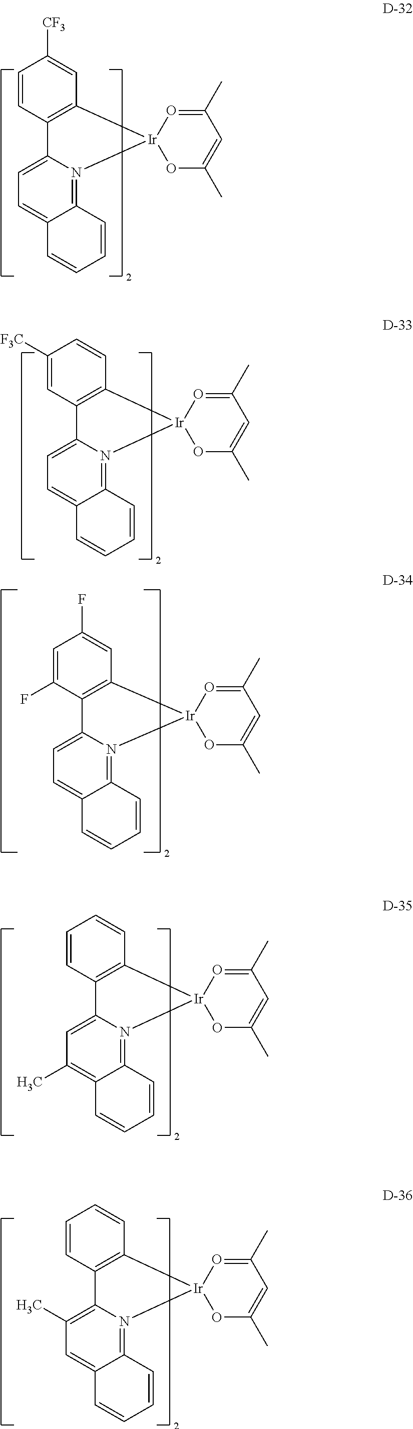

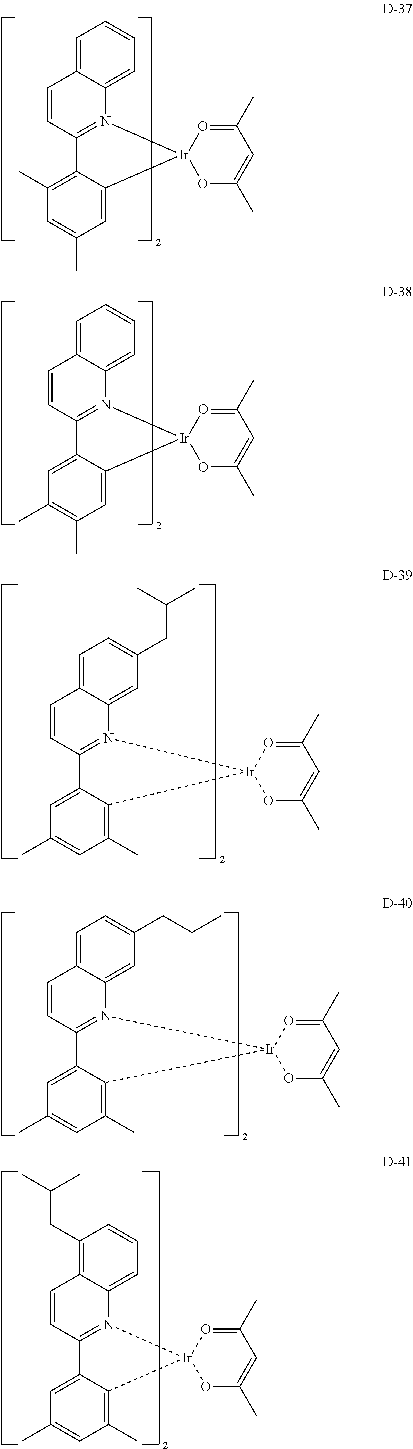

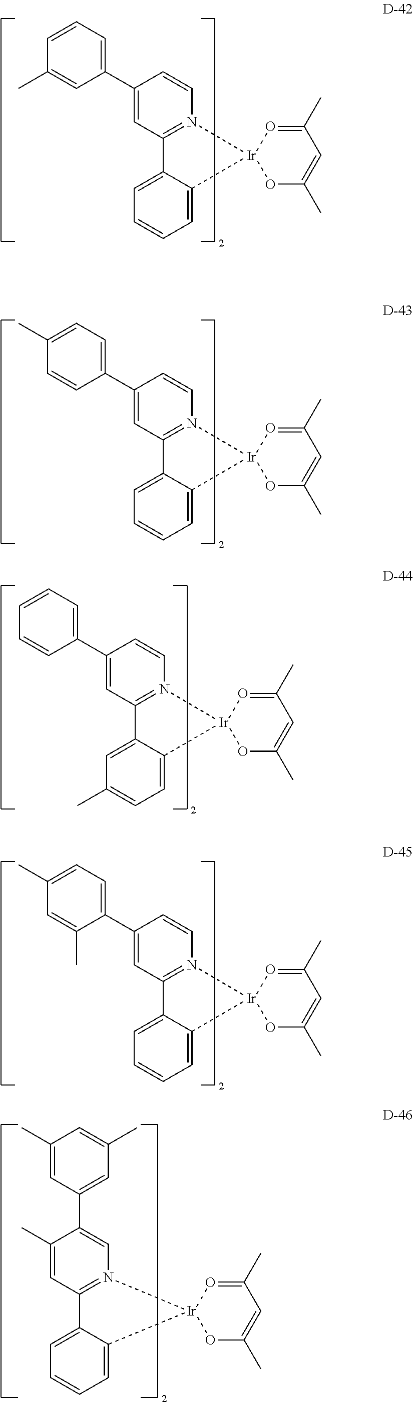

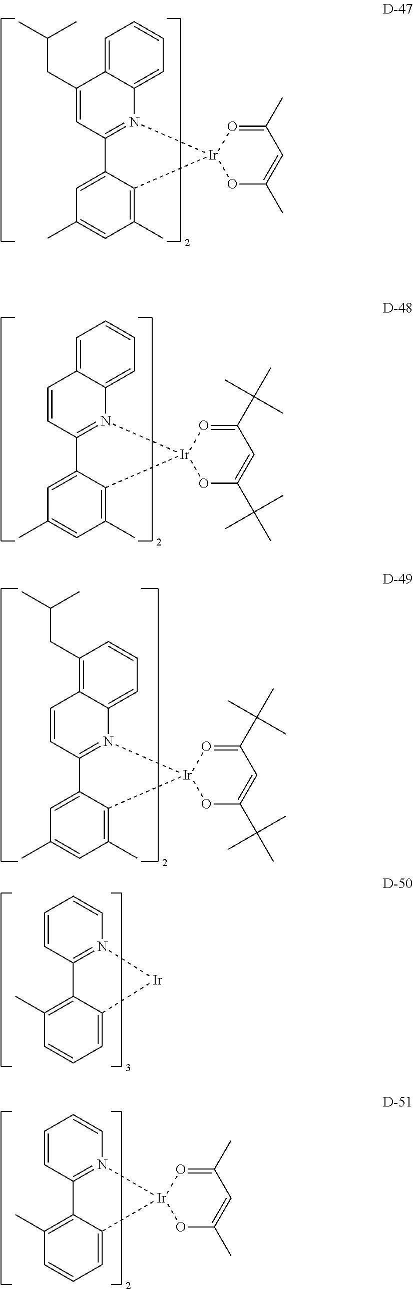

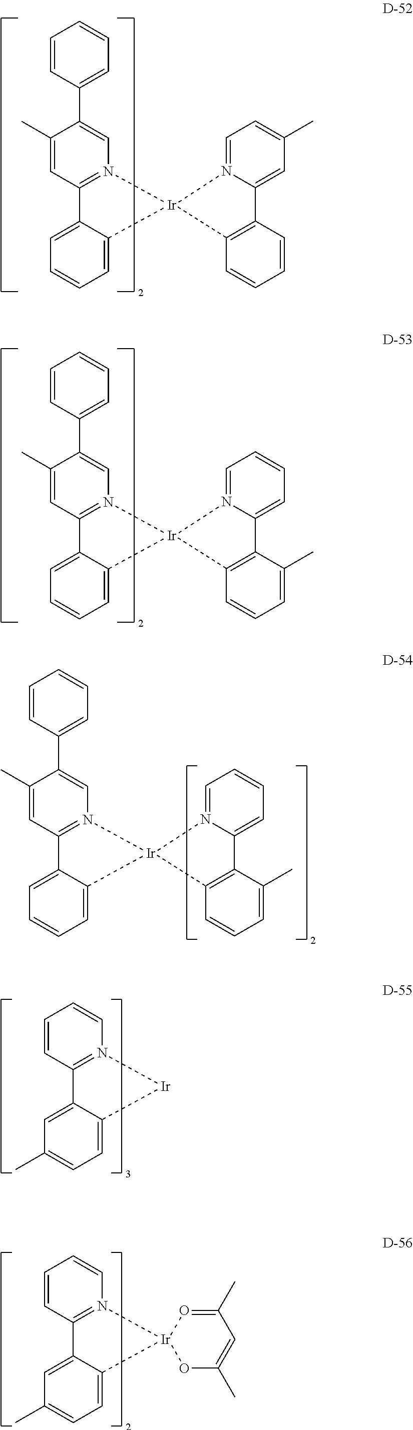

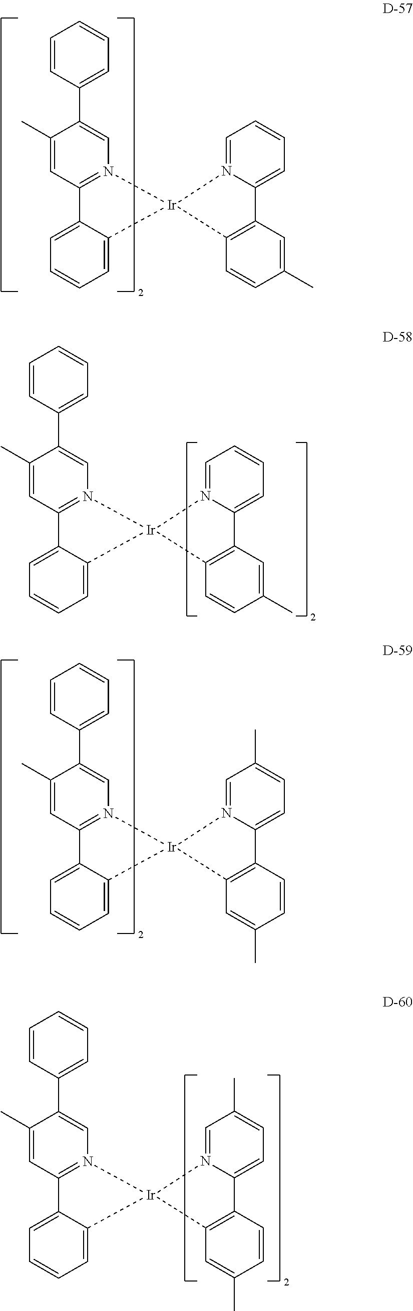

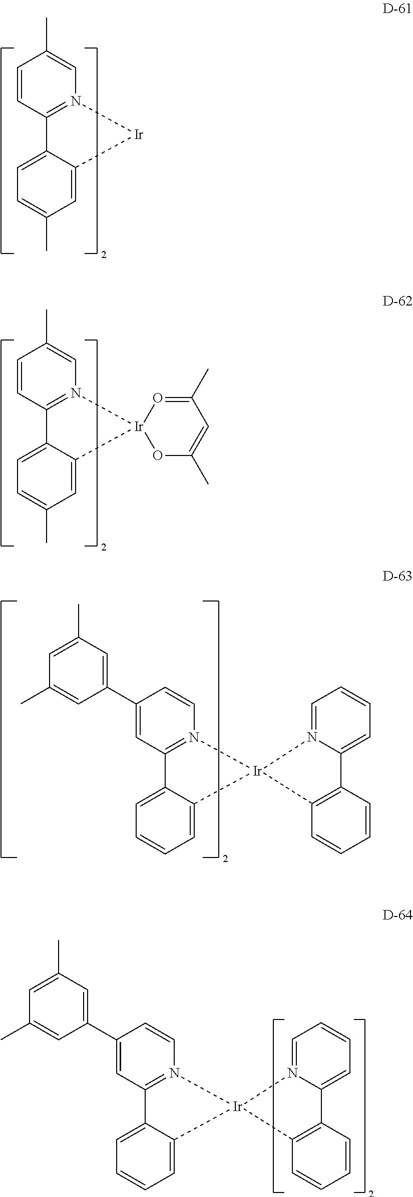

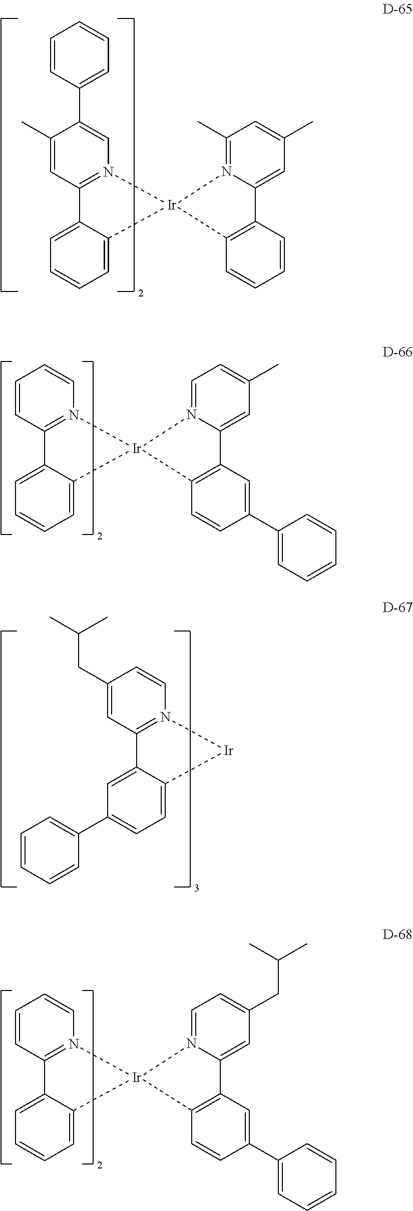

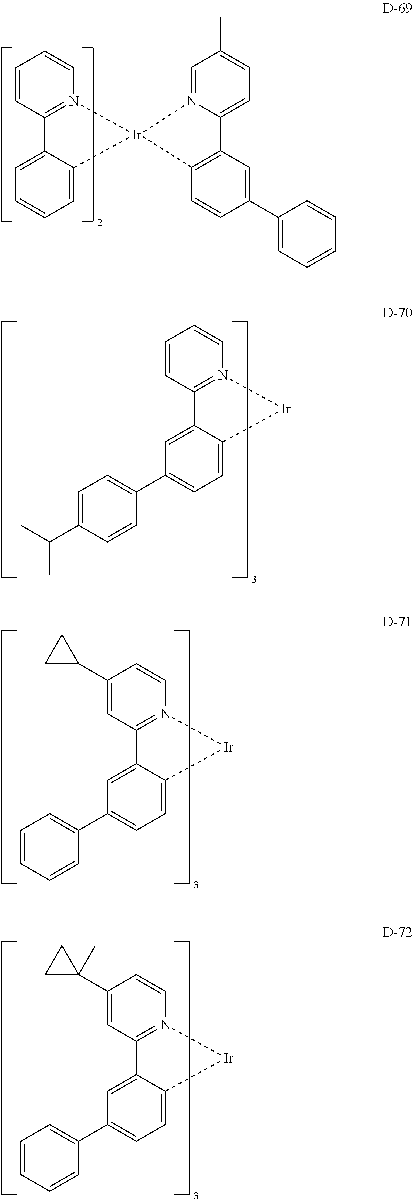

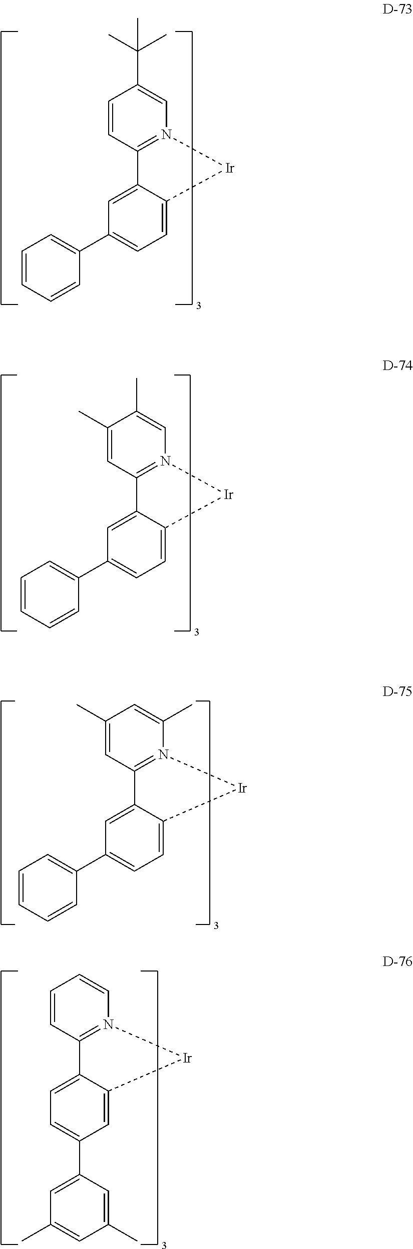

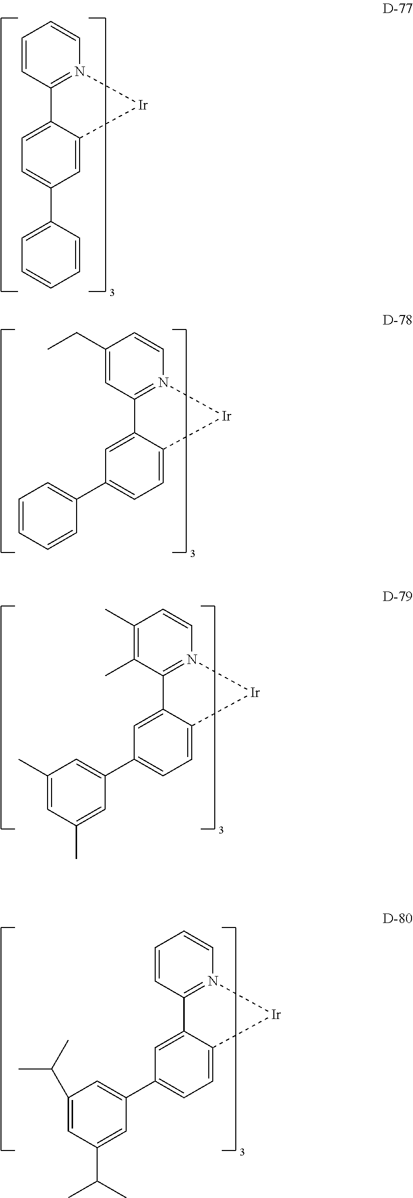

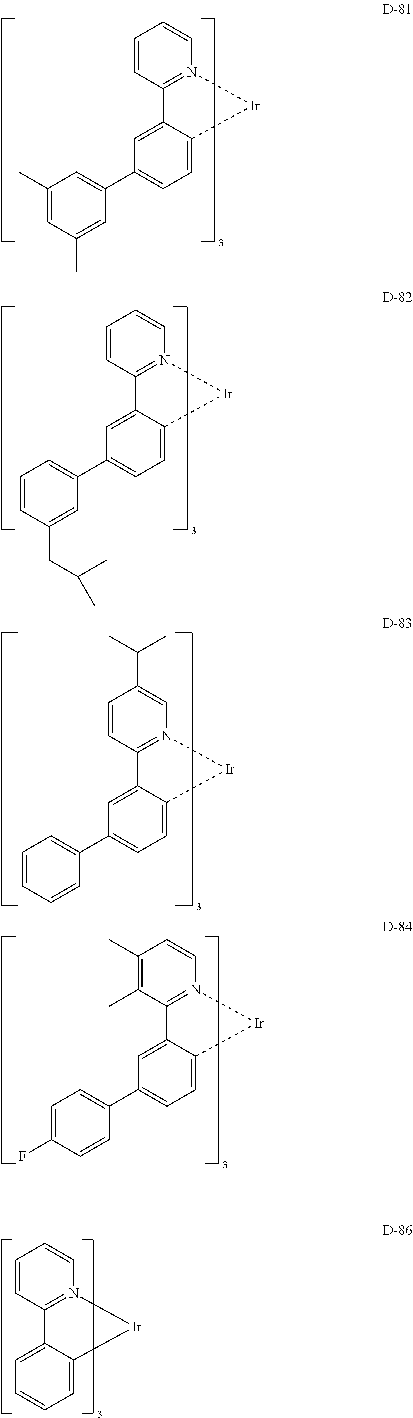

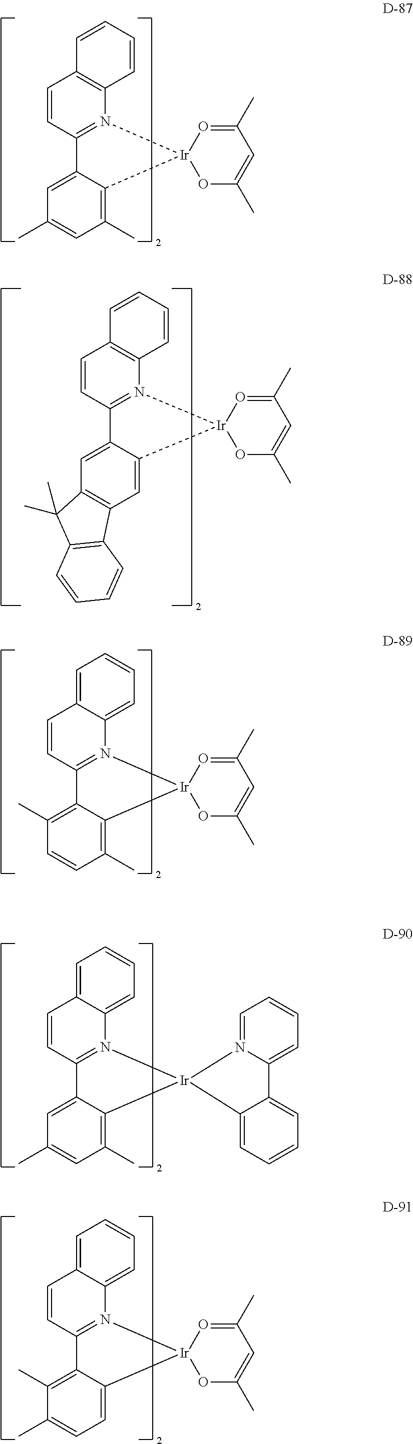

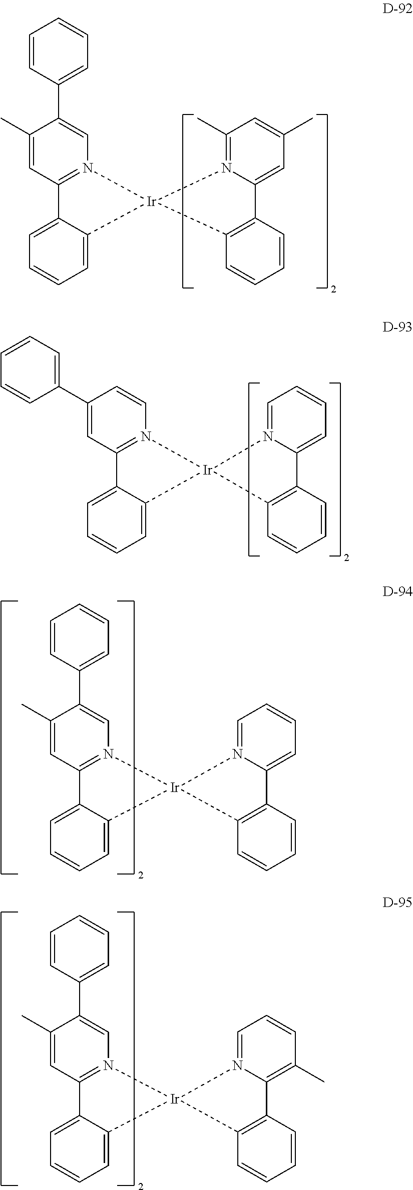

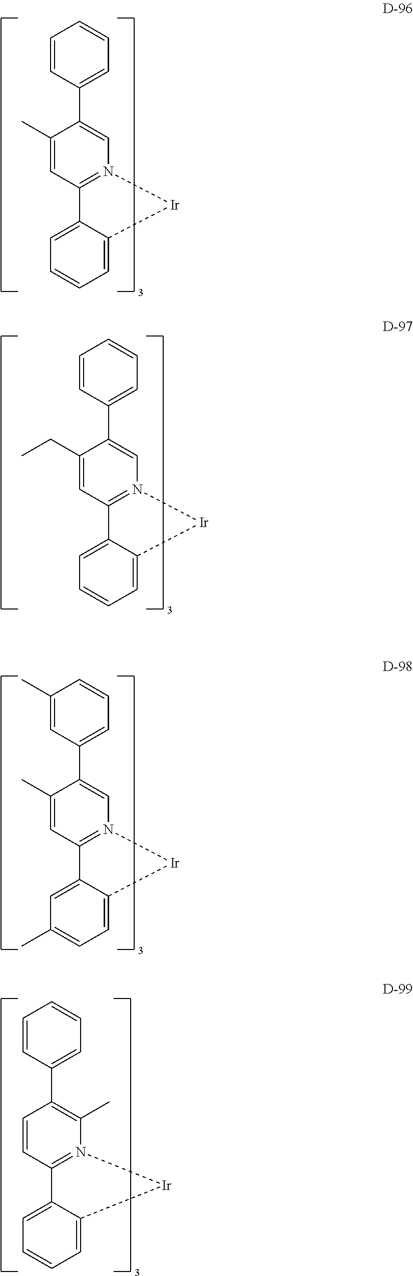

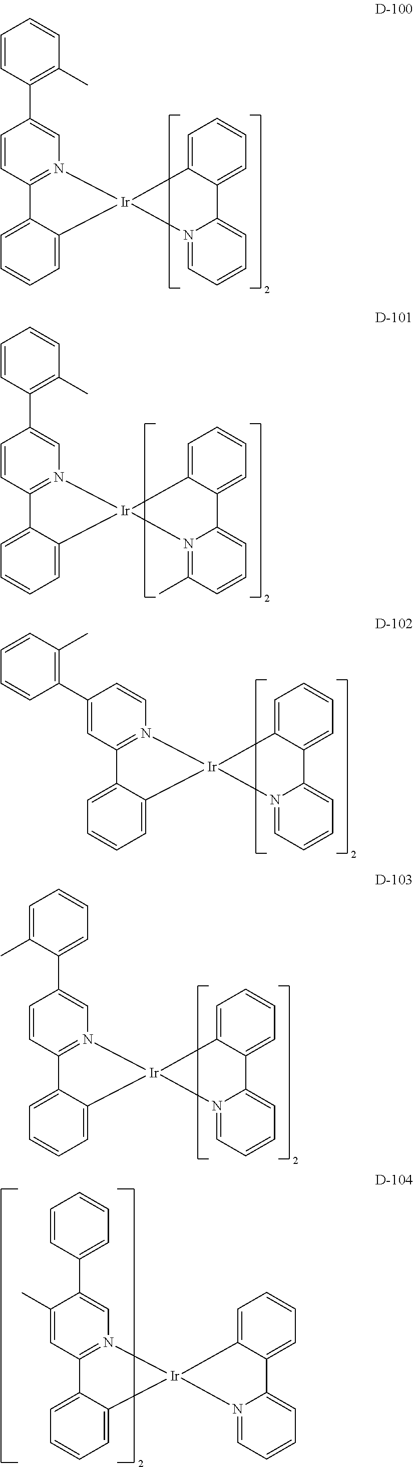

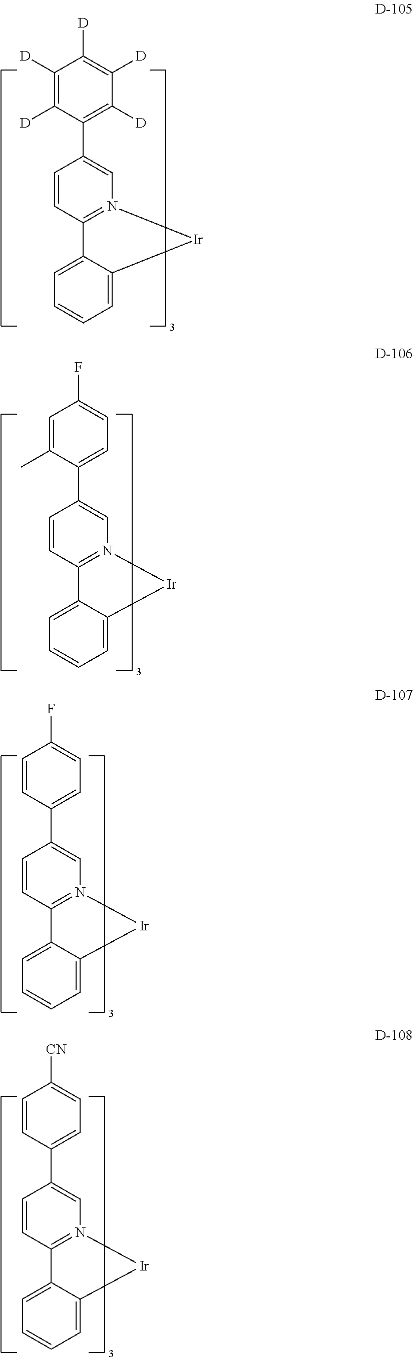

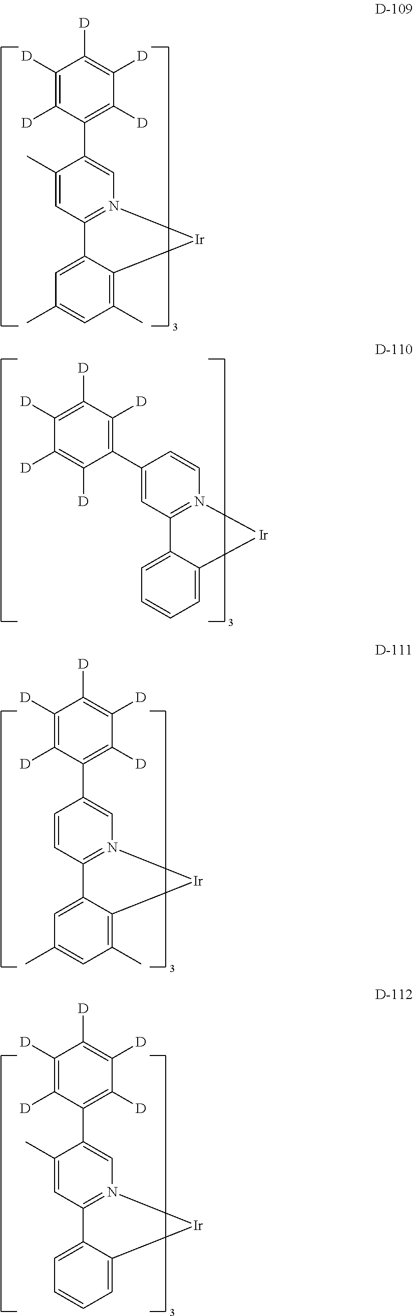

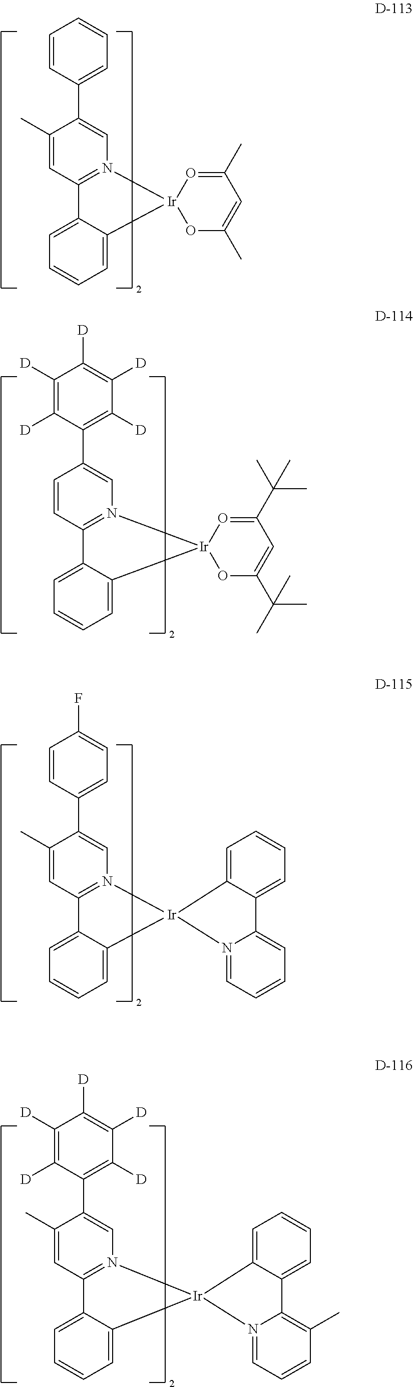

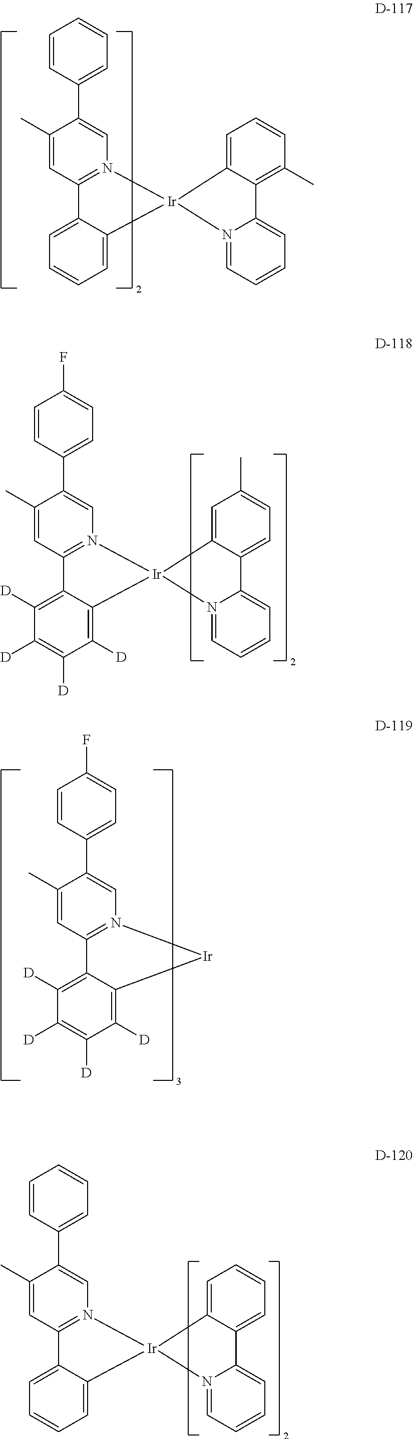

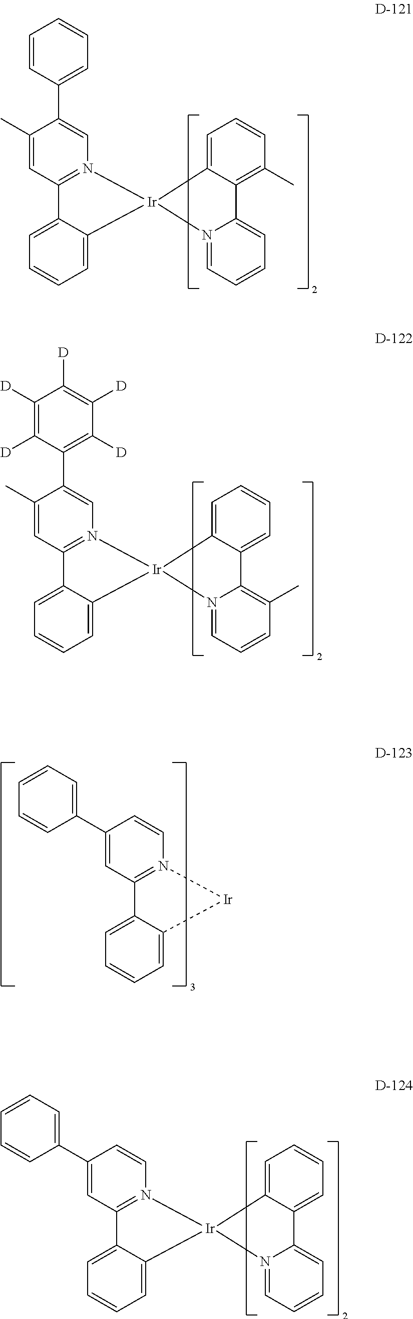

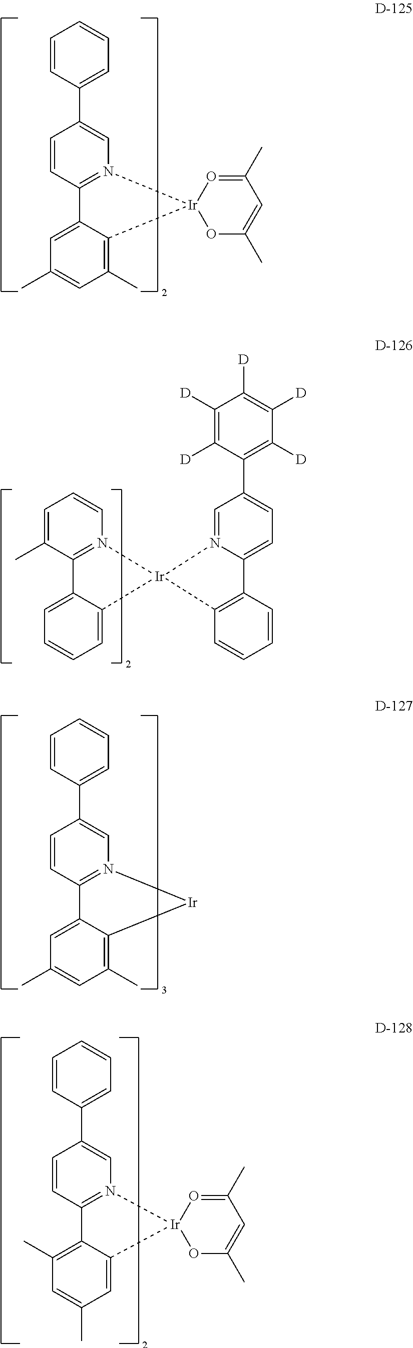

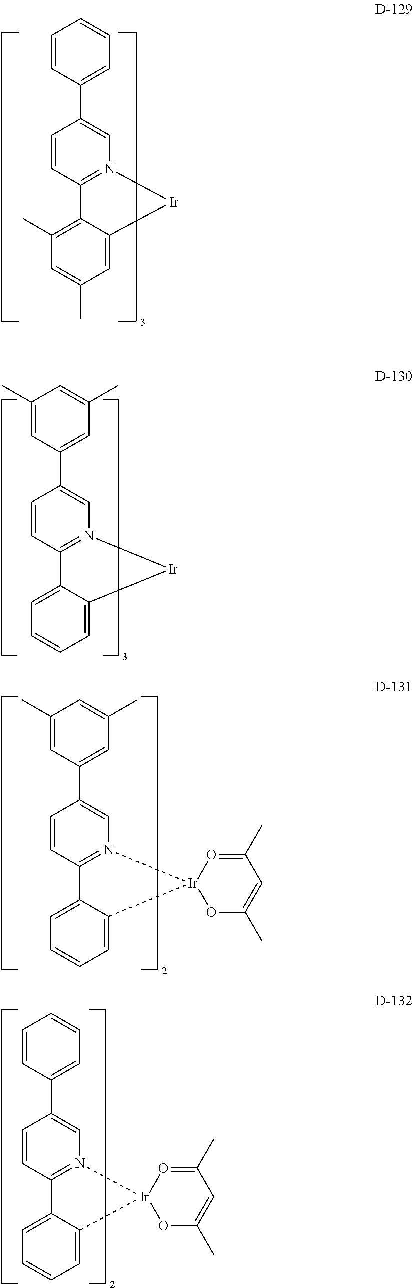

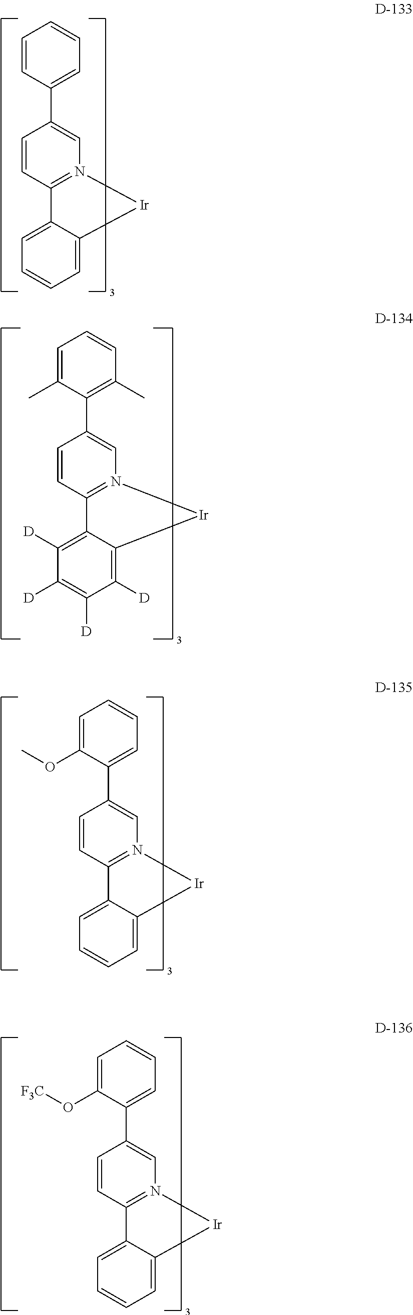

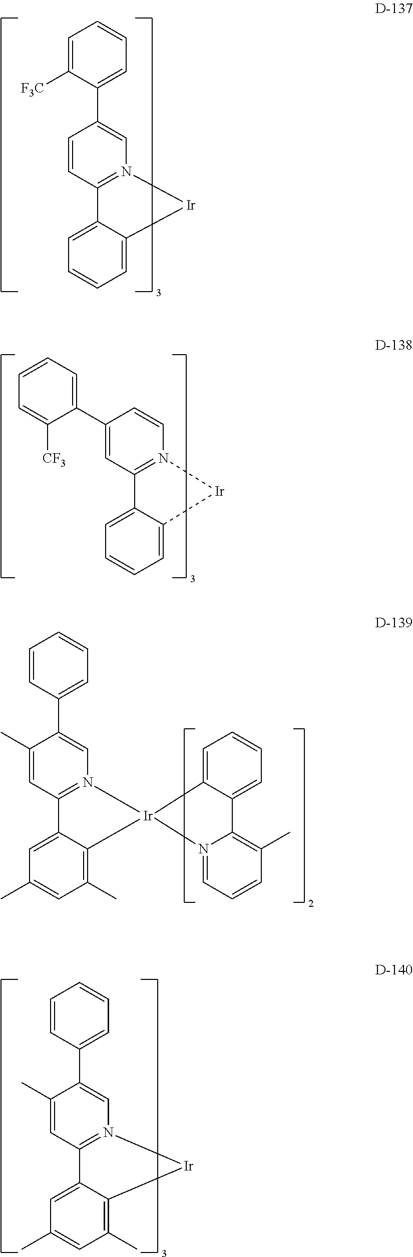

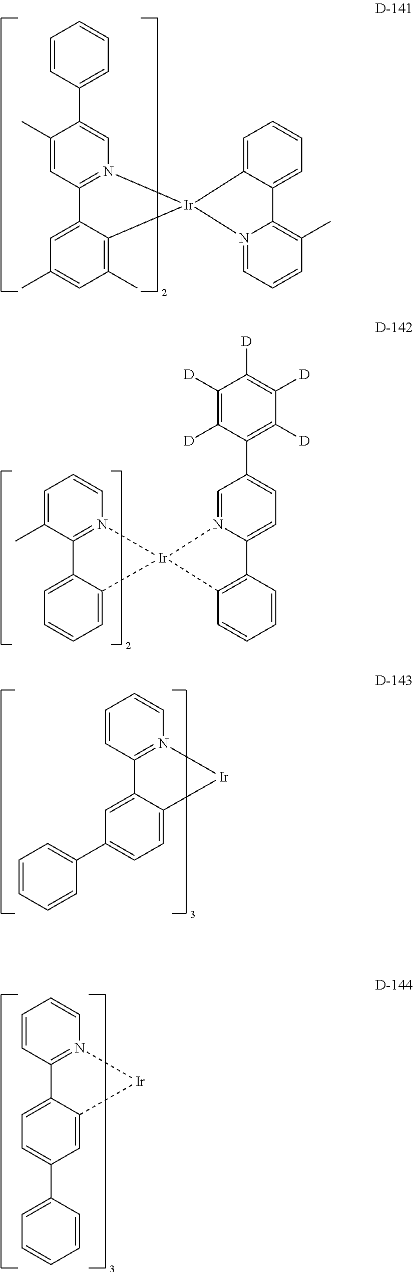

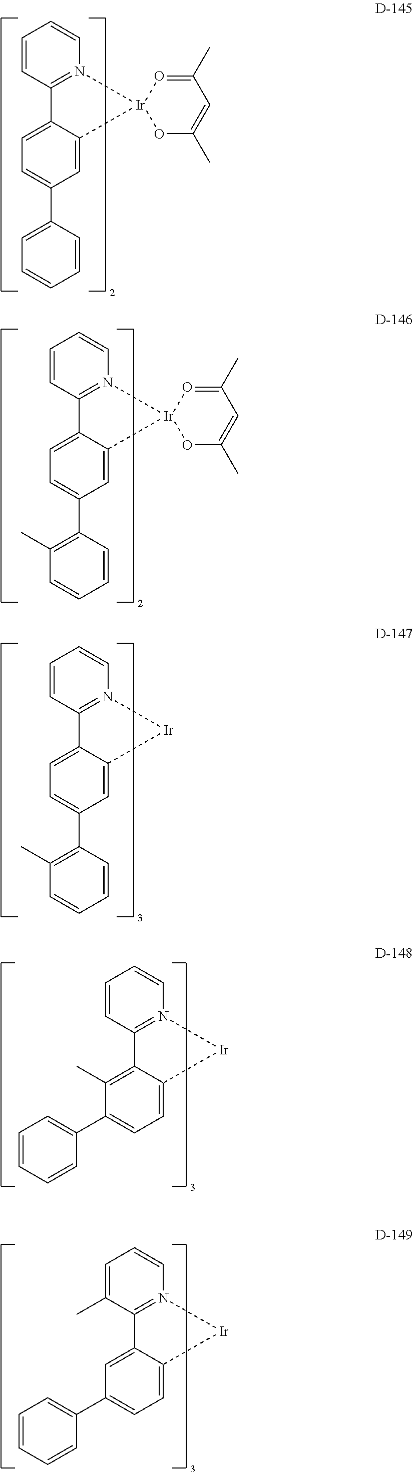

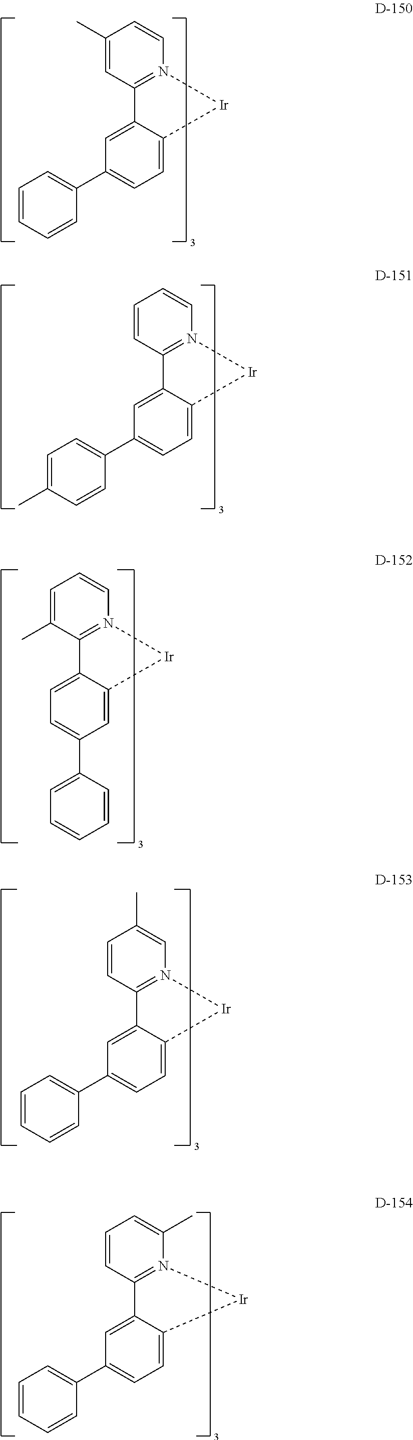

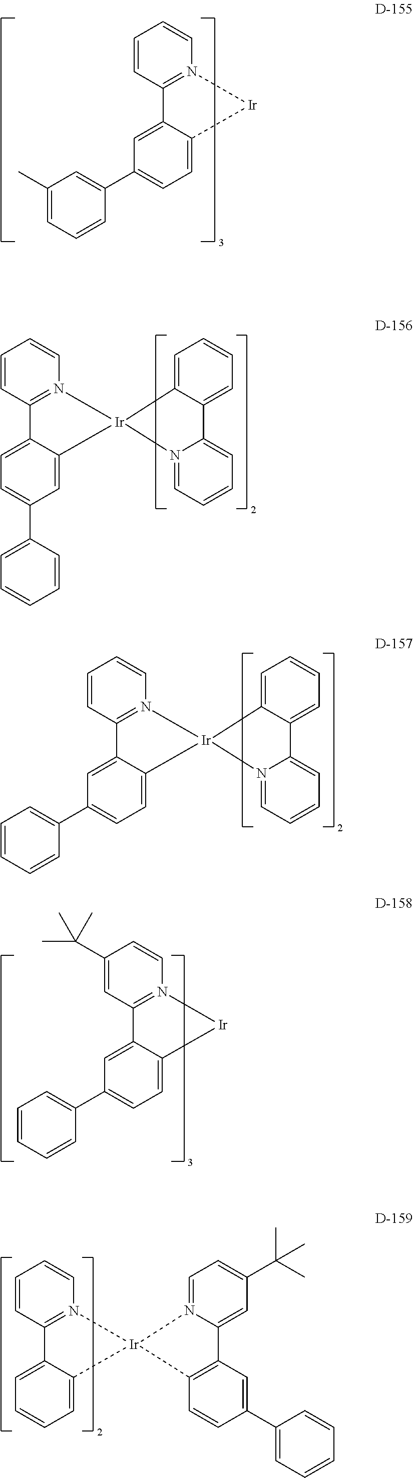

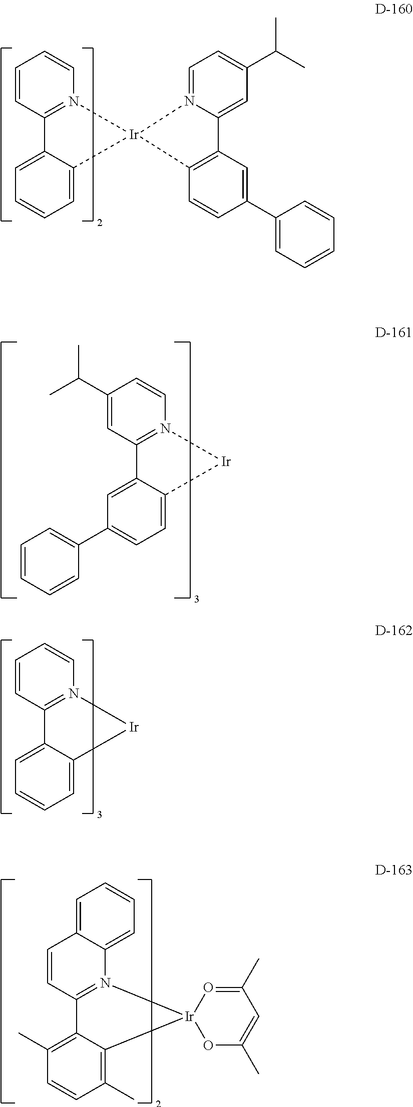

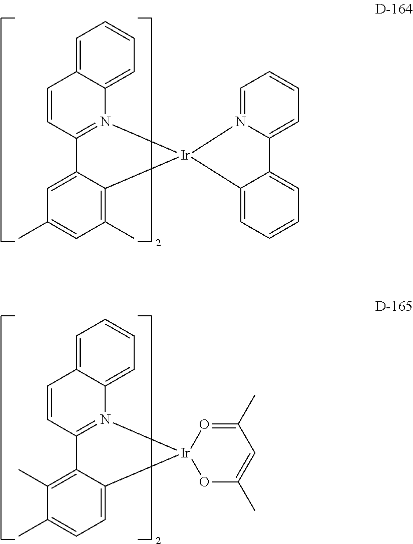

The organic electroluminescent device of the present invention may further comprise, in addition to the organic electroluminescent compounds represented by formula 1, at least one compound selected from the group consisting of arylamine-based compounds and styrylarylamine-based compounds.

In the organic electroluminescent device of the present invention, the organic layer may further comprise, in addition to the organic electroluminescent compounds represented by formula 1, at least one metal selected from the group consisting of metals of Group 1, metals of Group 2, transition metals of the 4^{th} period, transition metals of the 5^{th} period, lanthanides, and organic metals of d-transition elements of the Periodic Table, or at least one complex compound comprising the metal. The organic layer may further comprise a light-emitting layer or a charge generating layer.

In addition, the organic electroluminescent device of the present invention may emit white light by further comprising at least one light-emitting layer which comprises a blue electroluminescent compound, a red electroluminescent compound or a green electroluminescent compound which is known in the art, besides the organic electroluminescent compound of formula 1; and may further include a yellow or orange light-emitting layer, if necessary.

Preferably, in the organic electroluminescent device according to the present invention, at least one layer (hereinafter, “a surface layer”) selected from a chalcogenide layer, a metal halide layer and a metal oxide layer may be placed on an inner surface(s) of one or both electrode(s). Specifically, it is preferred that a chalcogenide (includes oxides) layer of silicon or aluminum is placed on an anode surface of an electroluminescent medium layer, and a metal halide layer or metal oxide layer is placed on a cathode surface of an electroluminescent medium layer. The surface layer provides operating stability for the organic electroluminescent device. Preferably, the chalcogenide includes SiO_{X}(1≦X≦2), AlO_{X}(1≦X≦1.5), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

Preferably, in the organic electroluminescent device according to the present invention, a mixed region of an electron transport compound and an reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Further, the hole transport compound is oxidized to a cation, and thus it becomes easier to inject and transport holes from the mixed region to the electroluminescent medium. Preferably, the oxidative dopant includes various Lewis acids and acceptor compounds; and the reductive dopant includes alkali metals, alkali metal compounds, alkaline earth metals, rare-earth metals, and mixtures thereof. A reductive dopant layer may be employed as a charge generating layer to prepare an organic electroluminescent device having two or more light-emitting layers and emitting white light.

In order to form each layer constituting the organic electroluminescent device according to the present invention, dry film-forming methods, such as vacuum evaporation, sputtering, plasma, ion plating methods, etc., or wet film-forming methods, such as spin coating, dip coating, flow coating methods, etc., can be used.

When using a wet film-forming method, a thin film is formed by dissolving or dispersing the material constituting each layer in suitable solvents, such as ethanol, chloroform, tetrahydrofuran, dioxane, etc. The solvents are not specifically limited as long as the material constituting each layer is soluble or dispersible in the solvents, which do not cause any problems in forming a layer.

Hereinafter, the organic electroluminescent compound of the present invention, the preparation method of the compound, and the luminescent properties of the device comprising the compound will be explained in detail with reference to the following examples:

### Example 1

### Preparation of Compound C-1

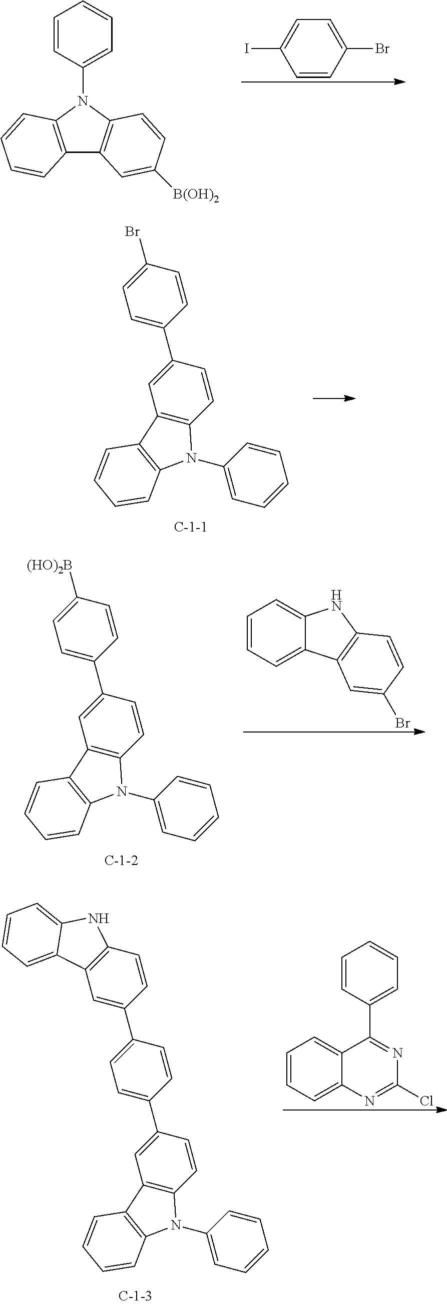

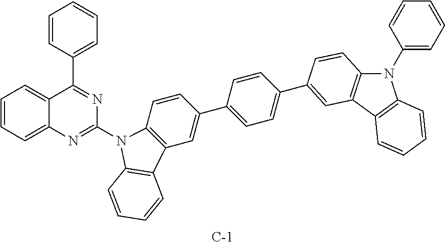

Preparation of Compound C-1-1

After adding N-phenylcarbazole-3-boronic acid (30.0 g, 105.0 mmol), 4-bromoiodobenzene (44.0 g, 157.0 mmol), tetrakis(triphenylphosphine)palladium(0) [Pd(PPh₃)₄] (3.5 g, 3.14 mmol), Na₂CO₃ (33.0 g, 313.0 mmol), toluene (600 mL), ethanol (EtOH) (150 mL) and distilled water (150 mL) to a 2 L round-bottom flask (RBF), the reaction mixture was stirred for 2 hours at 110° C. The reaction mixture was worked-up by using ethyl acetate (EA)/H₂O, was dried over MgSO₄ to remove moisture, was distilled under reduced pressure, and was separated through column chromatography by using methylene chloride (MC) and hexane to obtain compound C-1-1 (30.0 g, 72%) as a yellow solid.

Preparation of Compound C-1-2

After adding compound C-1-1 (30.0 g, 75.3 mmol) to a 1 L RBF and substituting it with nitrogen, tetrahydrofuran (THF) (400.0 mL) was added to the flask. The solution was cooled to −78° C., n-BuLi (36 mL, 2.5 M in hexane, 90.4 mmol) was added to the solution, and the mixture was stirred for one hour. After adding triisopropyl borate (26.0 mL, 113 mmol) to the mixture, the mixture was stirred for 24 hours. The mixture was quenched with 2M HCl, was extracted with EA/H₂O, was dried over MgSO₄ to remove moisture, was distilled under reduced pressure, and was recrystallized by using MC and hexane to obtain compound C-1-2 (22.0 g, 80%).

Preparation of Compound C-1-3

After adding compound C-1-2 (20.0 g, 55.0 mmol), 3-bromocarbazole (11.0 g, 44.7 mmol), Pd(PPh₃)₄ (2.6 g, 2.2 mmol), K₂CO₃ (15.0 g, 107.0 mmol), toluene (150 mL), EtOH (50 mL) and distilled water (50 mL) to a 1 L RBF, the reaction mixture was stirred for 24 hours at 110° C. The reaction mixture was extracted by using EA/H₂O, was dried over MgSO₄ to remove moisture, was distilled under reduced pressure, and was separated through column chromatography by using MC and hexane to obtain compound C-1-3 (15.0 g, 69%) as a yellow solid.

Preparation of Compound C-1

After adding compound C-1-3 (8.0 g, 16.5 mmol) and dimethylformamide (DMF) (200 mL) to a 250 mL RBF and dissolving the mixture by stirring, NaH (0.85 g, 60% dispersion in a mineral oil, 21.5 mmol) was added to the mixture and the reaction mixture was stirred for 30 minutes. 2-Chloro-4-phenylquinazoline (4.0 g, 16.5 mmol) was slowly added to the reaction mixture. After the addition, the reaction mixture was stirred for 2 hours at 50° C. The reaction mixture was quenched with methanol and was filtered to obtain a solid. The obtained solid was dried in a vacuum oven and was separated through column chromatography by using MC and hexane to obtain compound C-1 (6.7 g, 60%) as a yellow solid.

### Example 2

### Preparation of Compound C-76

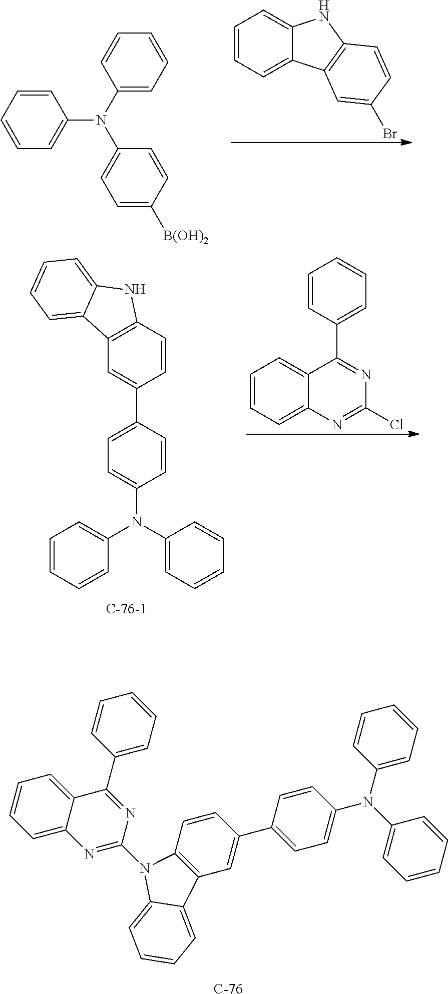

Preparation of Compound C-76-1

After adding 4-(diphenylamino)phenylboronic acid (14.0 g, 48.4 mmol), 3-bromocarbazole (10.0 g, 40.3 mmol), Pd(PPh₃)₄ (2.4 g, 2.0 mmol), K₂CO₃ (13.0 g, 96.8 mmol), toluene (200 mL), EtOH (50 mL) and distilled water (50 mL) to a 1 L RBF, the reaction mixture was stirred for 24 hours at 110° C. The reaction mixture was extracted by using EA/H₂O, was dried over MgSO₄ to remove moisture, was distilled under reduced pressure, and was separated through column chromatography by using MC and hexane to obtain compound C-76-1 (14.0 g, 84%) as a yellow solid.

Preparation of Compound C-76

Compound C-76-1 (6.0 g, 14.6 mmol) and DMF (75 mL) were added to a 250 mL RBF and were dissolved by stirring. NaH (0.9 g, 60% dispersion in a mineral oil, 21.9 mmol) was added to the mixture and the reaction mixture was stirred for 30 minutes. 2-Chloro-4-phenylquinazoline (4.0 g, 17.5 mmol) was slowly added to the reaction mixture, After the addition, the reaction mixture was stirred for 2 hours at 50° C. The reaction mixture was quenched with methanol and was filtered to obtain a solid. The obtained solid was dried in a vacuum oven and was separated through column chromatography by using MC and hexane to obtain compound C-76 (5.2 g, 58%) as a yellow solid.

### Example 3

### Preparation of Compound C-77

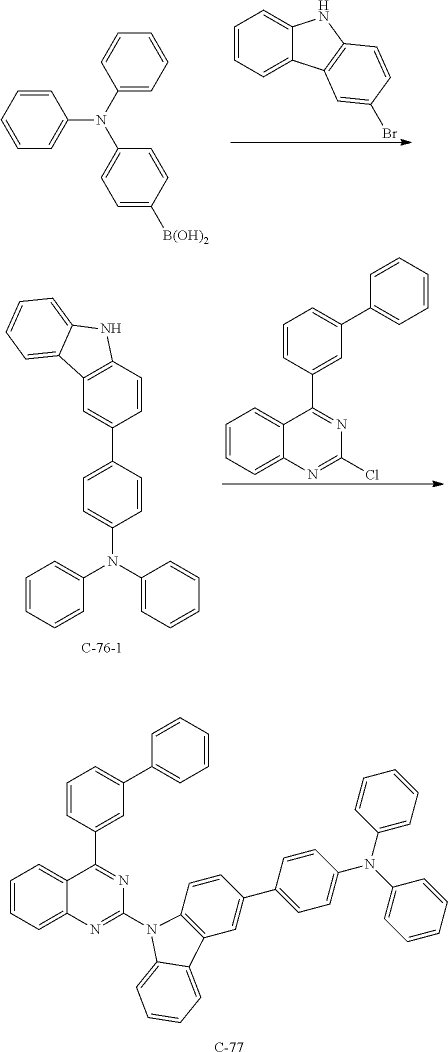

Compound C-76-1 (4.0 g, 10.2 mmol) and DMF (50 mL) were added to a 250 mL RBF and were dissolved by stirring. NaH (0.6 g, 60% dispersion in a mineral oil, 15.4 mmol) was added to the mixture and then the reaction mixture was stirred for 30 minutes. 4-([1,1′-biphenyl]-3-yl)-2-chloroquinazoline (4.0 g, 12.3 mmol) was slowly added to the reaction mixture. After the addition, the reaction mixture was stirred for 2 hours at 50° C. The reaction mixture was quenched with methanol and was filtered to obtain a solid. The obtained solid was dried in a vacuum oven, and was separated through column chromatography by using MC and hexane to obtain compound C-77 (2.1 g, 30%) as a yellow solid.

The physical properties of the compounds of the present invention, which were prepared in Examples 1 to 3, are provided in the Table 1 below:

TABLE 1

| | | PL | | | |
|---|---|---|---|---|---|
| Compound | Yield | (in Tol, | | MS/EIMS | |
| Nos. | (%) | nm) | MP (° C.) | measured | calculated |
| C-1 | 60 | 489 | 257 | 688.20 | 719.26 |
| C-76 | 58 | 535 | 236 | 614.05 | 614.25 |
| C-77 | 30 | 543 | 200 | 690.04 | 690.28 |

### Device Example 1

### Production of an OLED Device Using the Organic Electroluminescent Compound According to the Present Invention

An OLED device was produced using the light-emitting material according to the present invention. A transparent electrode indium tin oxide (ITO) thin film (15 Ω/sq) on a glass substrate for an organic light-emitting diode (OLED) device (Samsung Corning, Republic of Korea) was subjected to an ultrasonic washing with trichloroethylene, acetone, ethanol and distilled water, sequentially, and then was stored in isopropanol. Then, the ITO substrate was mounted on a substrate holder of a vacuum vapor depositing apparatus. N¹,N^{1′}-([1,1′-biphenyl]-4,4′-diyl)bis(N¹-(naphthalen-1-yl)-N⁴,N⁴-diphenylbenzene-1,4-diamine) was introduced into a cell of the vacuum vapor depositing apparatus, and then the pressure in the chamber of the apparatus was controlled to 10⁻⁶ torr. Thereafter, an electric current was applied to the cell to evaporate the introduced material, thereby forming a hole injection layer having a thickness of 60 nm on the ITO substrate. Then, N,N′-di(4-biphenyl)-N,N′-di(4-biphenyl)-4,4′-diaminobiphenyl was introduced into another cell of the vacuum vapor depositing apparatus, and was evaporated by applying electric current to the cell, thereby forming a hole transport layer having a thickness of 20 nm on the hole injection layer. Thereafter, compound C-1 of the present invention as a host was introduced into one cell of the vacuum vapor depositing apparatus, and compound D-11 as a dopant was introduced into another cell. The two materials were evaporated at different rates and deposited in a doping amount of 4 wt % of the dopant, based on the total weight of the host and dopant, to form a light-emitting layer having a thickness of 30 nm on the hole transport layer. Then, 2-(4-(9,10-di(naphthalen-2-yl)anthracen-2-yl)phenyl)-1-phenyl-1H-benzo[d]imidazole was introduced into one cell and lithium quinolate (Liq) was introduced into another cell. The two materials were evaporated at the same rate and were respectively deposited in a doping amount of 50 wt % to form an electron transport layer having a thickness of 30 nm on the light-emitting layer. Then, after depositing lithium quinolate as an electron injection layer having a thickness of 2 nm on the electron transport layer, an Al cathode having a thickness of 150 nm was deposited by another vacuum vapor deposition apparatus on the electron injection layer. Thus, an OLED device was produced. All the materials used for producing the OLED device were purified by vacuum sublimation at 10⁻⁶ torr prior to use.

The produced OLED device showed red emission having a luminance of 1020 cd/m² and a current density of 11.8 mA/cm² at a driving voltage of 4.2 V. Further, the time taken to be reduced to 90% of the luminance at a luminance of 5,000 nit was at least 120 hours.

### Device Example 2

### Production of an OLED Device Using the Organic Electroluminescent Compound According to the Present Invention

An OLED device was produced in the same manner as in Device Example 1, except for using compound C-76 of the present invention as a host and compound D-7 as a dopant in a light-emitting material.

The produced OLED device showed red emission having a luminance of 1040 cd/m² and a current density of 10.2 mA/cm² at a driving voltage of 4.5 V. Further, the time taken to be reduced to 90% of the luminance at a luminance of 5,000 nit was at least 80 hours.

### Device Example 3

### Production of an OLED Device Using the Organic Electroluminescent Compounds According to the Present Invention

An OLED device was produced in the same manner as in Device Example 1, except for using compound C-77 of the present invention as a host and compound D-11 as a dopant in a light-emitting material.

The produced OLED device showed red emission having a luminance of 1050 cd/m² and a current density of 15.5 mA/cm² at a driving voltage of 4.7 V. Further, the time taken to be reduced to 90% of the luminance at a luminance of 5,000 nit was at least 50 hours.

### Comparative Example 1

### Production of an OLED Device Using a Conventional Light-Emitting Material

An OLED device was produced in the same manner as in Device Example 1, except that a light-emitting layer having a thickness of 30 nm was deposited on the hole transport layer by using 4,4′-N,N′-dicarbazol-biphenyl as a host and compound D-11 as a dopant in a light-emitting material and a hole blocking layer having a thickness of 10 nm was deposited on the light-emitting by using aluminum(III) bis(2-methyl-8-quinolinato)-4-phenyl phenolate.

The produced OLED device showed red emission having a luminance of 1000 cd/m² and a current density of 20.0 mA/cm² at a driving voltage of 8.2 V. Further, the time taken to be reduced to 90% of the luminance at a luminance of 5,000 nit was at least 10 hours.

The organic electroluminescent compounds according to the present invention have high electron transmission efficiency, and thus can prevent crystallization in the production of a device; and are suitable for forming a layer, and thus can improve the current features of the device thereby reducing driving voltage of the device. The organic electroluminescent device having enhanced power efficiency can be prepared by using the organic electroluminescent compounds according to the present invention.

1. An organic electroluminescent compound represented by the following formula 1: 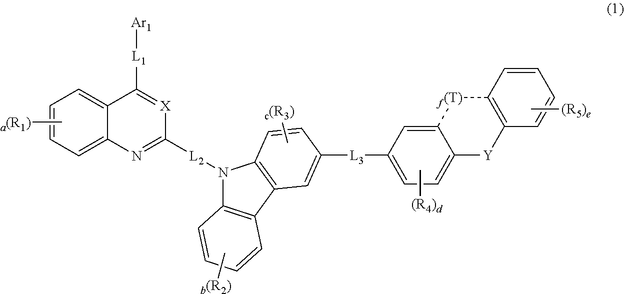
wherein
X represents CH or N;
L₁ and L₂ each independently represent a single bond, a substituted or unsubstituted 5- to 30-membered heteroarylene group, or a substituted or unsubstituted (C6-C30)arylene group;
L₃ represents a single bond, a substituted or unsubstituted (C1-C30)alkylene group, a substituted or unsubstituted (C6-C30)arylene group, or a substituted or unsubstituted 3- to 30-membered heteroarylene group;
Y represents —O—, —S—, —CR₁₁R₁₂— or —NR₁₃—;
T represents a chemical bond;
Ar₁ represents hydrogen, a halogen, deuterium, a substituted or unsubstituted 5- to 30-membered heteroaryl group, a substituted or unsubstituted (C6-C30)aryl group, or a substituted or unsubstituted (C1-C30)alkyl group;
R₁ to R₅ each independently represent hydrogen, deuterium, a halogen, a substituted or unsubstituted (C1-C30)alkyl group, a substituted or unsubstituted (C6-C30)aryl group, a substituted or unsubstituted 3- to 30-membered heteroaryl group, a substituted or unsubstituted (C3-C30)cycloalkyl group, a substituted or unsubstituted 5- to 7-membered heterocycloalkyl group, a substituted or unsubstituted (C6-C30)aryl(C1-C30)alkyl group, a substituted or unsubstituted (C6-C30)aryl group which is fused with at least one (C3-C30) aliphatic ring, a 5- to 7-membered heterocycloalkyl group which is fused with at least one substituted or unsubstituted (C6-C30) aromatic ring, a (C3-C30)cycloalkyl group which is fused with at least one substituted or unsubstituted (C6-C30) aromatic ring, —NR₁₄R₁₅, —SiR₁₆R₁₇R₁₈, —SR₁₉, —OR₂₀, a (C2-C30)alkenyl group, a (C2-C30)alkynyl group, a cyano group, a nitro group, or a hydroxyl group;
R₁₁ to R₂₀ are as defined in R₁ to R₅;
a, b and e each independently represent an integer of 1 to 4; where a, b or e is an integer of 2 or more, each of R₁, each of R₂ or each of R₅ is the same or different;
c and d each independently represent an integer of 1 to 3; where c or d is an integer of 2 or more, each of R₃ or each of R₄ is the same or different;
f represents an integer of 0 or 1; where f is 0, Y represents —NR₁₃—, wherein R₁₃ may be linked to R₅ to form a mono- or polycyclic, (C5-C30) alicyclic or aromatic ring whose carbon atom(s) may be replaced with at least one hetero atom selected from nitrogen, oxygen and sulfur; and
the heterocycloalkyl group and heteroaryl(ene) group contain at least one hetero atom selected from B, N, O, S, P(═O), Si and P.

2. The organic electroluminescent compound according to claim 1, wherein the substituents of the substituted alkyl(ene) group, the substituted aryl(ene) group, the substituted heteroaryl(ene) group, the substituted cycloalkyl group, and the substituted heterocycloalkyl group in L₁ to L₃, Ar₁, R₁ to R₅, and R₁₁ to R₂₀ each independently are at least one selected from the group consisting of deuterium; a halogen; a (C1-C30)alkyl group which is unsubstituted or substituted with a halogen; a (C6-C30)aryl group; a 3- to 30-membered heteroaryl group which is unsubstituted or substituted with a (C6-C30)aryl group; a 5- to 7-membered heterocycloalkyl group; a 5- to 7-membered heterocycloalkyl group which is fused with at least one (C6-C30) aromatic ring; a (C3-C30)cycloalkyl group; a (C6-C30)cycloalkyl group which is fused with at least one (C6-C30) aromatic ring; RₐR_{b}R_{c}Si—; a (C2-C30)alkenyl group; a (C2-C30)alkynyl group; a cyano group; a carbazolyl group; —NR_{d}Rₑ; —BR_{f}R_{g}; —PRₕRᵢ; —P(═O)RⱼRₖ; (C6-C30)aryl(C1-C30)alkyl group; (C1-C30)alkyl(C6-C30)aryl group; RₗZ—; RₘC(═O)—; RₘC(═O)O—; a carboxyl group; a nitro group; and a hydroxyl group, in which Rₐ to Rₗ each independently represent a (C1-C30)alkyl group, a (C6-C30)aryl group or a 3- to 30-membered heteroaryl group, or are linked to an adjacent substituent(s) to form a mono- or polycyclic, (C5-C30) alicyclic or aromatic ring whose carbon atom(s) may be replaced with at least one hetero atom selected from nitrogen, oxygen and sulfur, Z represents S or O, and Rₘ represents a (C1-C30)alkyl group, a (C1-C30)alkoxy group, a (C6-C30)aryl group or a (C6-C30)aryloxy group.

3. The organic electroluminescent compound according to claim 1, wherein L₁ and L₂ each independently represent a single bond, a substituted or unsubstituted 5- to 15-membered heteroarylene group, or a substituted or unsubstituted (C6-C15)arylene group;
L₃ represents a single bond, or a substituted or unsubstituted (C6-C15)arylene group;
Y represents —O—, —S—, —CR₁₁R₁₂— or —NR₁₃—, in which R₁₁ and R₁₂ each independently represent a substituted or unsubstituted (C1-C10)alkyl group, R₁₃ represents a substituted or unsubstituted (C6-C15)aryl group, or a substituted or unsubstituted 5- to 15-membered heteroaryl group;
T represents a single bond;
Ar₁ represents hydrogen, a substituted or unsubstituted 5- to 20-membered heteroaryl group, a substituted or unsubstituted (C6-C20)aryl group, or a substituted or unsubstituted (C1-C10)alkyl group; and
R₁ to R₅ each independently represent hydrogen, a substituted or unsubstituted (C6-C15)aryl group, or a substituted or unsubstituted 5- to 15-membered heteroaryl group.

4. The organic electroluminescent compound according to claim 3, wherein L₁ and L₂ each independently represent a single bond, an unsubstituted 5- to 15-membered heteroarylene group, or a (C6-C15)arylene group which is unsubstituted or substituted with a (C1-C6) alkyl group;
L₃ represents a single bond or an unsubstituted (C6-C15)arylene group;
Y represents —O—, —S—, —CR₁₁R₁₂— or —NR₁₃—, in which R₁₁ and R₁₂ each independently represent an unsubstituted (C1-C10)alkyl group, R₁₃ represents a (C6-C15)aryl group which is unsubstituted or substituted with deuterium, a halogen, a (C1-C6)alkyl group or a (C6-C15)aryl group, or a 5- to 15-membered heteroaryl group which is substituted with a (C6-C15)aryl group;
Ar₁ represents hydrogen; a 5- to 20-membered heteroaryl group which is unsubstituted or substituted with a (C6-C15)aryl group; a (C6-C20)aryl group which is unsubstituted or substituted with deuterium, a halogen, a (C1-C6)alkyl group, a (C6-C15)aryl group or a 5- to 15-membered heteroaryl group; or a (C1-C10)alkyl group which is unsubstituted or substituted with a (C1-C6)alkyl group; and
R₁ to R₅ each independently represent hydrogen, an unsubstituted (C6-C15)aryl group or an unsubstituted 5- to 15-membered heteroaryl group.

5. The organic electroluminescent compound according to claim 1, wherein the compound represented by formula 1 is selected from the group consisting of the following compounds:

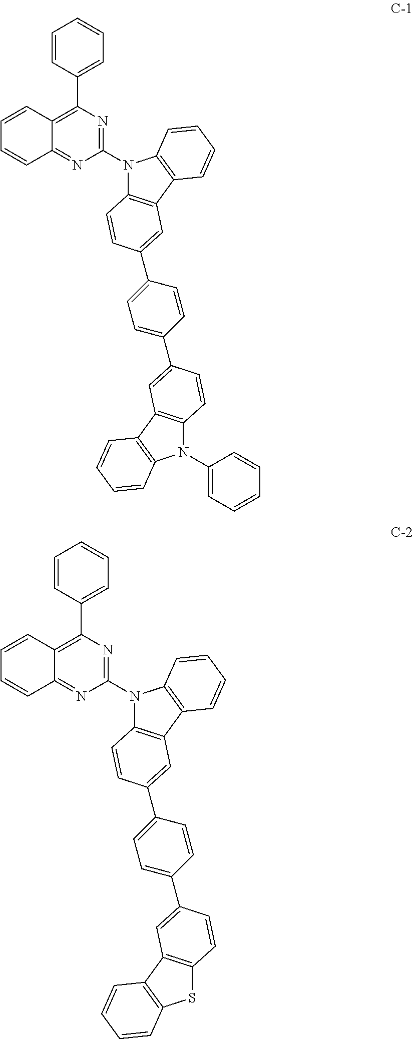 [SMILES: c1ccc(-c2nc(-n3c4ccccc4c4cc(-c5ccc(-c6ccc7c(c6)c6ccccc6n7-c6ccccc6)cc5)ccc43)nc3ccccc23)cc1 ; c1ccc(-c2nc(-n3c4ccccc4c4cc(-c5ccc(-c6ccc7sc8ccccc8c7c6)cc5)ccc43)nc3ccccc23)cc1]

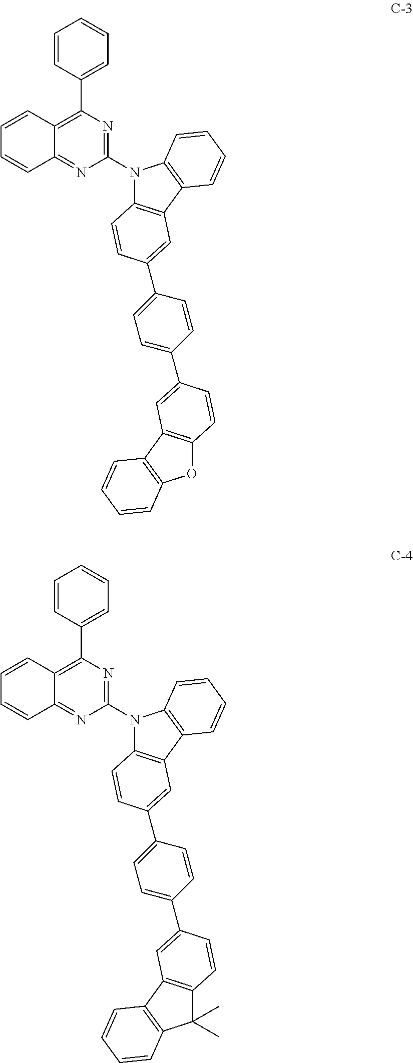 [SMILES: c1ccc(-c2nc(-n3c4ccccc4c4cc(-c5ccc(-c6ccc7oc8ccccc8c7c6)cc5)ccc43)nc3ccccc23)cc1 ; CC1(C)c2ccccc2-c2cc(-c3ccc(-c4ccc5c(c4)c4ccccc4n5-c4nc(-c5ccccc5)c5ccccc5n4)cc3)ccc21]

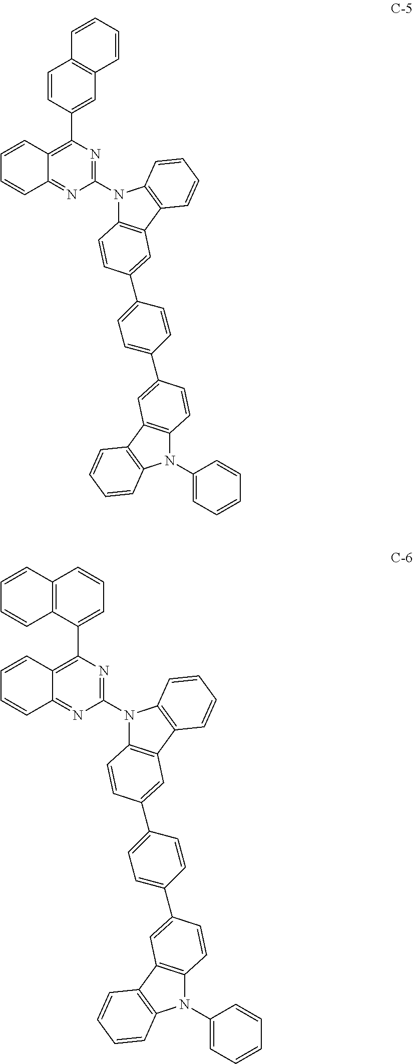 [SMILES: c1ccc(-n2c3ccccc3c3cc(-c4ccc(-c5ccc6c(c5)c5ccccc5n6-c5nc(-c6ccc7ccccc7c6)c6ccccc6n5)cc4)ccc32)cc1 ; c1ccc(-n2c3ccccc3c3cc(-c4ccc(-c5ccc6c(c5)c5ccccc5n6-c5nc(-c6cccc7ccccc67)c6ccccc6n5)cc4)ccc32)cc1]

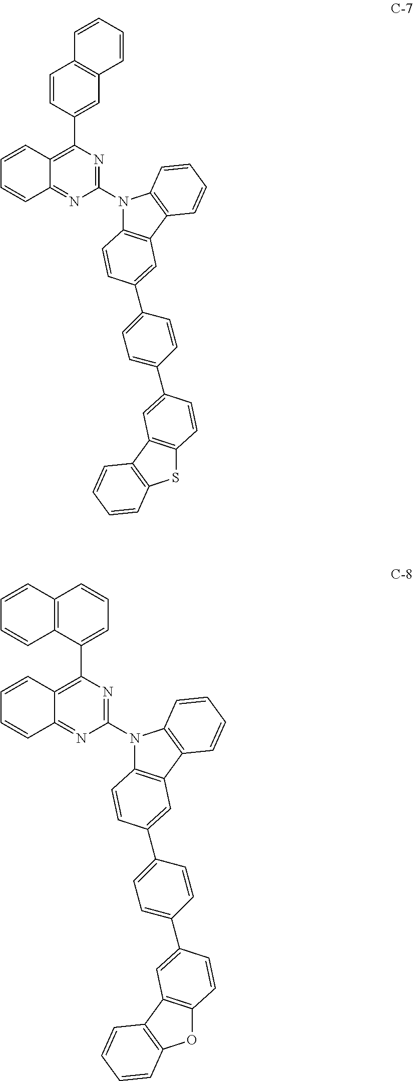 [SMILES: c1ccc2cc(-c3nc(-n4c5ccccc5c5cc(-c6ccc(-c7ccc8sc9ccccc9c8c7)cc6)ccc54)nc4ccccc34)ccc2c1 ; c1ccc2c(-c3nc(-n4c5ccccc5c5cc(-c6ccc(-c7ccc8oc9ccccc9c8c7)cc6)ccc54)nc4ccccc34)cccc2c1]

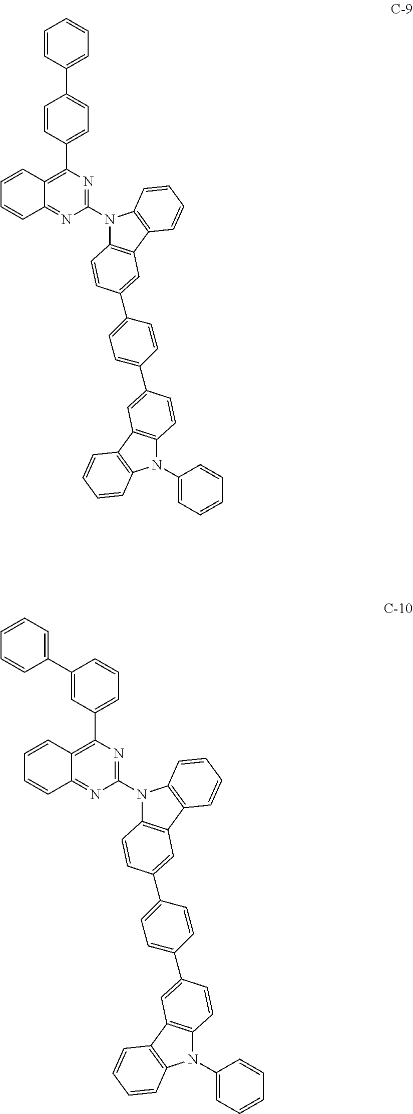 [SMILES: c1ccc(-c2ccc(-c3nc(-n4c5ccccc5c5cc(-c6ccc(-c7ccc8c(c7)c7ccccc7n8-c7ccccc7)cc6)ccc54)nc4ccccc34)cc2)cc1 ; c1ccc(-c2cccc(-c3nc(-n4c5ccccc5c5cc(-c6ccc(-c7ccc8c(c7)c7ccccc7n8-c7ccccc7)cc6)ccc54)nc4ccccc34)c2)cc1]

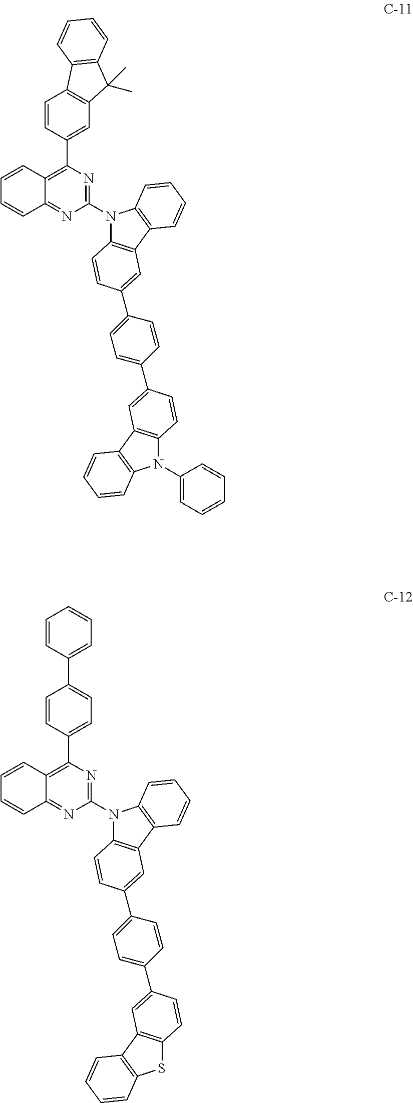 [SMILES: CC1(C)c2ccccc2-c2ccc(-c3nc(-n4c5ccccc5c5cc(-c6ccc(-c7ccc8c(c7)c7ccccc7n8-c7ccccc7)cc6)ccc54)nc4ccccc34)cc21 ; c1ccc(-c2ccc(-c3nc(-n4c5ccccc5c5cc(-c6ccc(-c7ccc8sc9ccccc9c8c7)cc6)ccc54)nc4ccccc34)cc2)cc1]

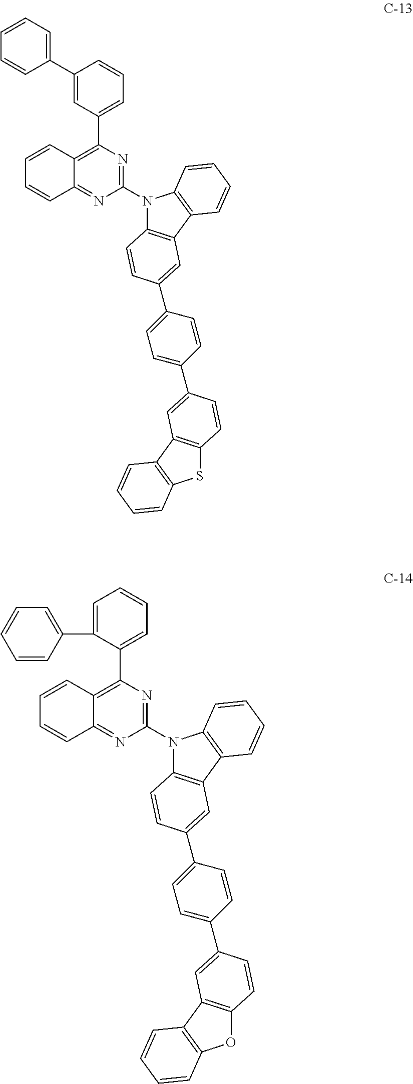 [SMILES: c1ccc(-c2cccc(-c3nc(-n4c5ccccc5c5cc(-c6ccc(-c7ccc8sc9ccccc9c8c7)cc6)ccc54)nc4ccccc34)c2)cc1 ; c1ccc(-c2ccccc2-c2nc(-n3c4ccccc4c4cc(-c5ccc(-c6ccc7oc8ccccc8c7c6)cc5)ccc43)nc3ccccc23)cc1]

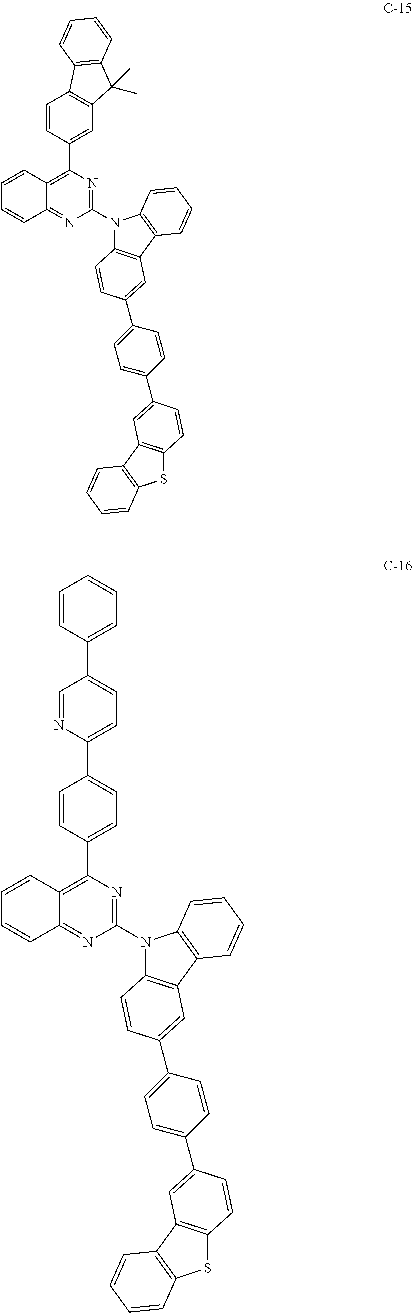 [SMILES: CC1(C)c2ccccc2-c2ccc(-c3nc(-n4c5ccccc5c5cc(-c6ccc(-c7ccc8sc9ccccc9c8c7)cc6)ccc54)nc4ccccc34)cc21 ; c1ccc(-c2ccc(-c3ccc(-c4nc(-n5c6ccccc6c6cc(-c7ccc(-c8ccc9sc%10ccccc%10c9c8)cc7)ccc65)nc5ccccc45)cc3)nc2)cc1]

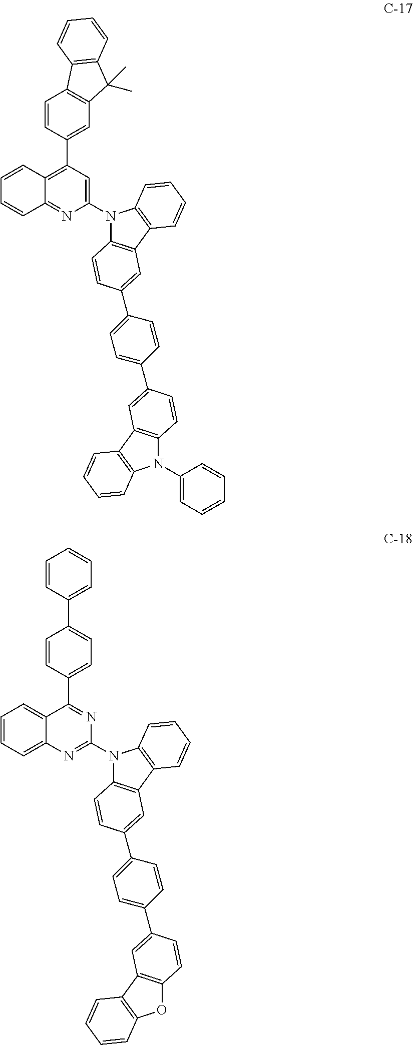 [SMILES: CC1(C)c2ccccc2-c2ccc(-c3cc(-n4c5ccccc5c5cc(-c6ccc(-c7ccc8c(c7)c7ccccc7n8-c7ccccc7)cc6)ccc54)nc4ccccc34)cc21 ; c1ccc(-c2ccc(-c3nc(-n4c5ccccc5c5cc(-c6ccc(-c7ccc8oc9ccccc9c8c7)cc6)ccc54)nc4ccccc34)cc2)cc1]

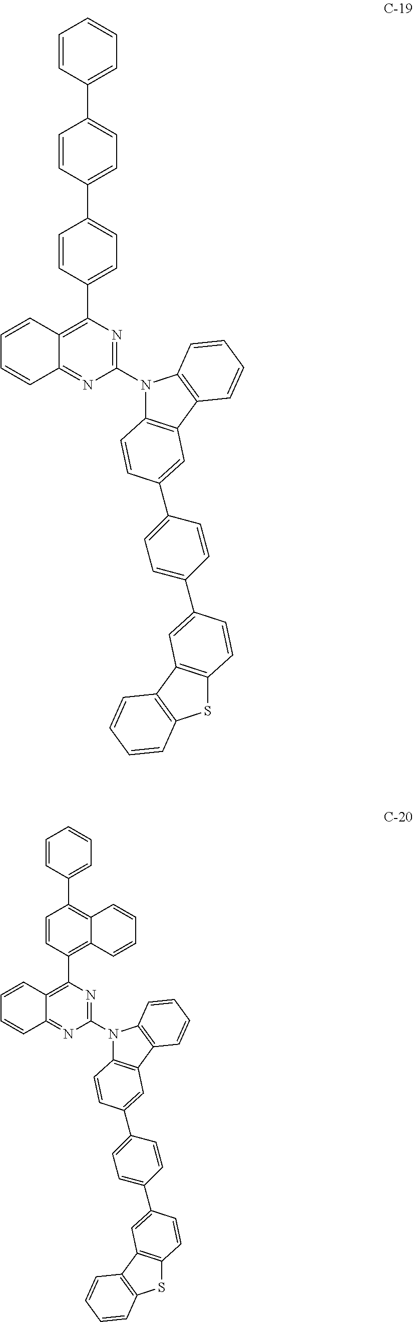 [SMILES: c1ccc(-c2ccc(-c3ccc(-c4nc(-n5c6ccccc6c6cc(-c7ccc(-c8ccc9sc%10ccccc%10c9c8)cc7)ccc65)nc5ccccc45)cc3)cc2)cc1 ; c1ccc(-c2ccc(-c3nc(-n4c5ccccc5c5cc(-c6ccc(-c7ccc8sc9ccccc9c8c7)cc6)ccc54)nc4ccccc34)c3ccccc23)cc1]

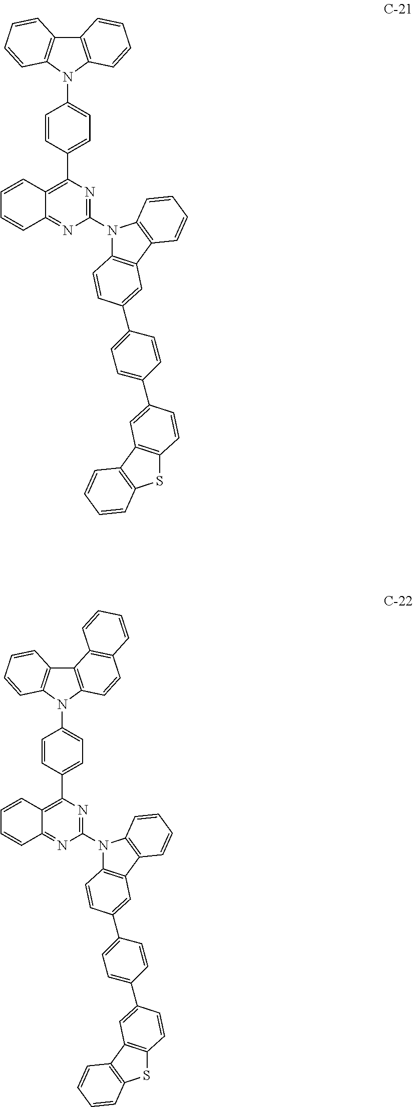 [SMILES: c1ccc2c(-c3ccc(-n4c5ccccc5c5ccccc54)cc3)nc(-n3c4ccccc4c4cc(-c5ccc(-c6ccc7sc8ccccc8c7c6)cc5)ccc43)nc2c1 ; c1ccc2c(c1)ccc1c2c2ccccc2n1-c1ccc(-c2nc(-n3c4ccccc4c4cc(-c5ccc(-c6ccc7sc8ccccc8c7c6)cc5)ccc43)nc3ccccc23)cc1]

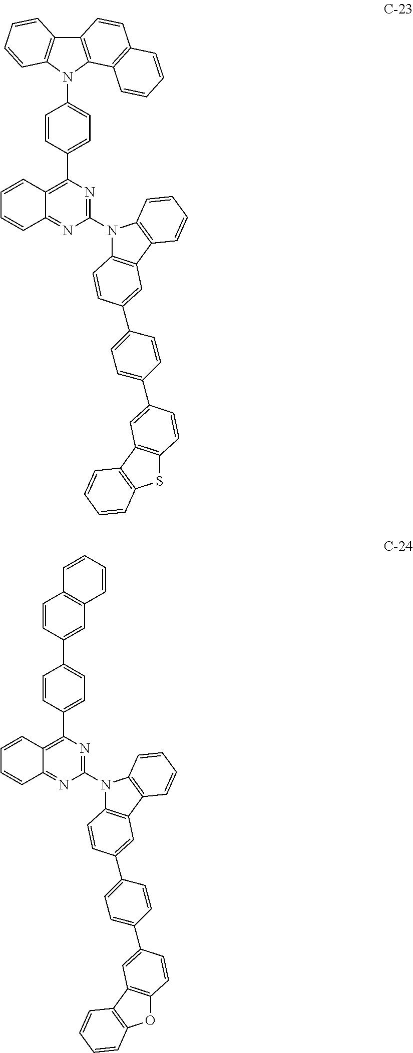 [SMILES: c1ccc2c(c1)ccc1c3ccccc3n(-c3ccc(-c4nc(-n5c6ccccc6c6cc(-c7ccc(-c8ccc9sc%10ccccc%10c9c8)cc7)ccc65)nc5ccccc45)cc3)c21 ; c1ccc2cc(-c3ccc(-c4nc(-n5c6ccccc6c6cc(-c7ccc(-c8ccc9oc%10ccccc%10c9c8)cc7)ccc65)nc5ccccc45)cc3)ccc2c1]

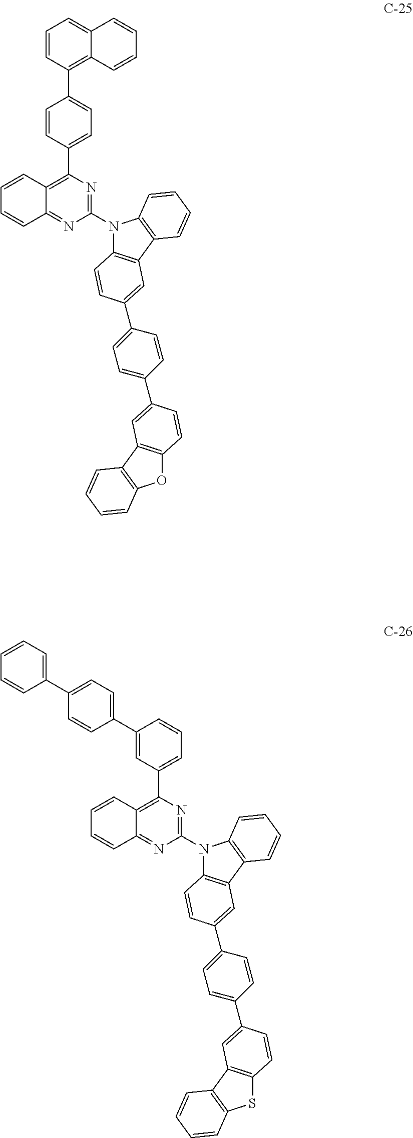 [SMILES: c1ccc2c(-c3ccc(-c4nc(-n5c6ccccc6c6cc(-c7ccc(-c8ccc9oc%10ccccc%10c9c8)cc7)ccc65)nc5ccccc45)cc3)cccc2c1 ; c1ccc(-c2ccc(-c3cccc(-c4nc(-n5c6ccccc6c6cc(-c7ccc(-c8ccc9sc%10ccccc%10c9c8)cc7)ccc65)nc5ccccc45)c3)cc2)cc1]

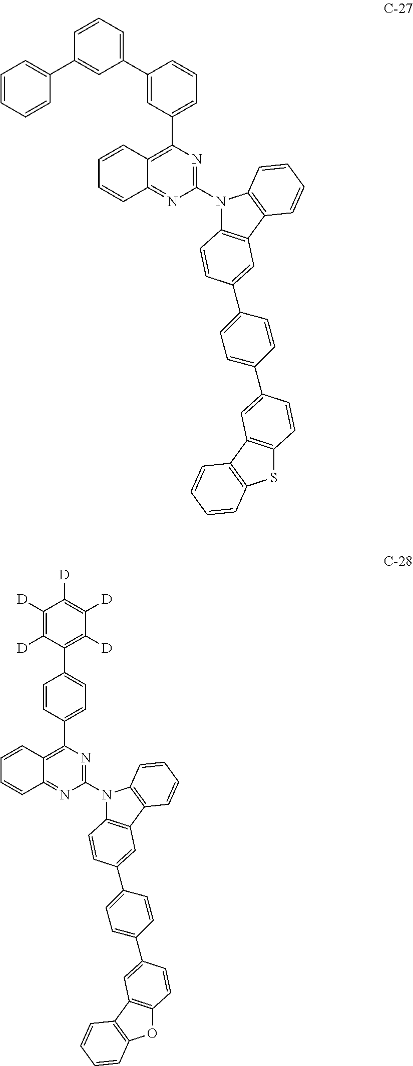 [SMILES: c1ccc(-c2cccc(-c3cccc(-c4nc(-n5c6ccccc6c6cc(-c7ccc(-c8ccc9sc%10ccccc%10c9c8)cc7)ccc65)nc5ccccc45)c3)c2)cc1 ; [2H]c1c([2H])c([2H])c(-c2ccc(-c3nc(-n4c5ccccc5c5cc(-c6ccc(-c7ccc8oc9ccccc9c8c7)cc6)ccc54)nc4ccccc34)cc2)c([2H])c1[2H]]

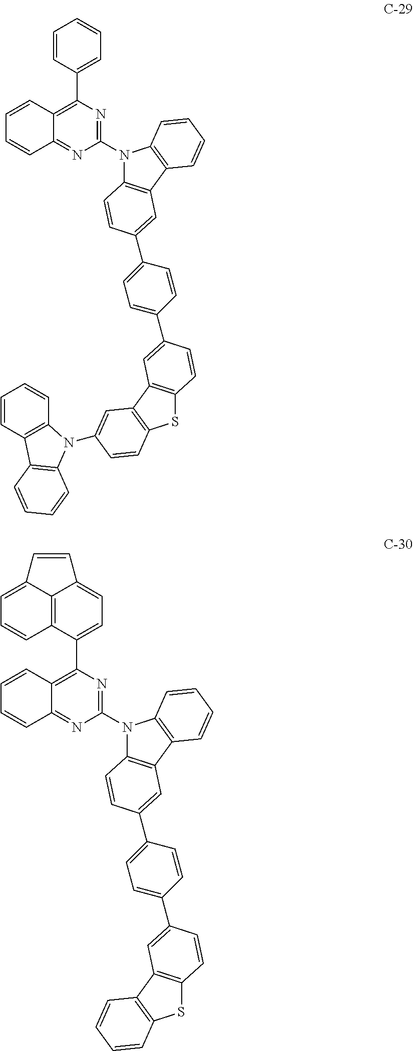 [SMILES: c1ccc(-c2nc(-n3c4ccccc4c4cc(-c5ccc(-c6ccc7sc8ccc(-n9c%10ccccc%10c%10ccccc%109)cc8c7c6)cc5)ccc43)nc3ccccc23)cc1 ; C1=Cc2ccc(-c3nc(-n4c5ccccc5c5cc(-c6ccc(-c7ccc8sc9ccccc9c8c7)cc6)ccc54)nc4ccccc34)c3cccc1c23]

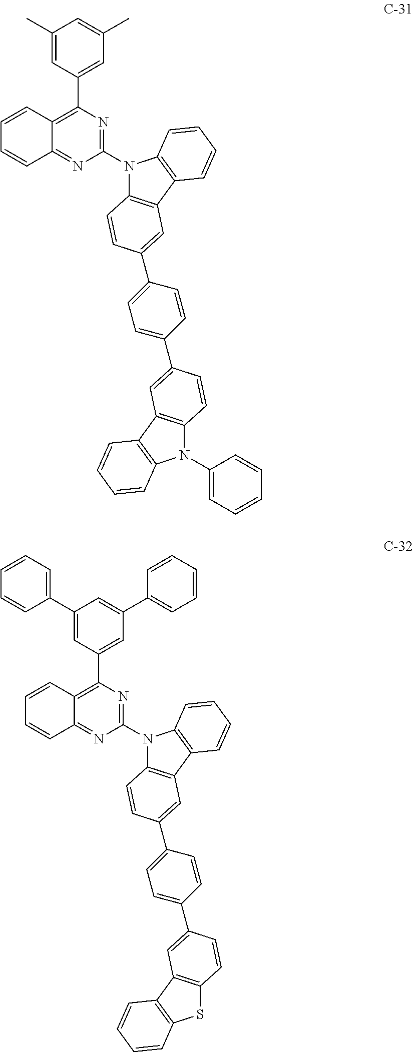 [SMILES: Cc1cc(C)cc(-c2nc(-n3c4ccccc4c4cc(-c5ccc(-c6ccc7c(c6)c6ccccc6n7-c6ccccc6)cc5)ccc43)nc3ccccc23)c1 ; c1ccc(-c2cc(-c3ccccc3)cc(-c3nc(-n4c5ccccc5c5cc(-c6ccc(-c7ccc8sc9ccccc9c8c7)cc6)ccc54)nc4ccccc34)c2)cc1]

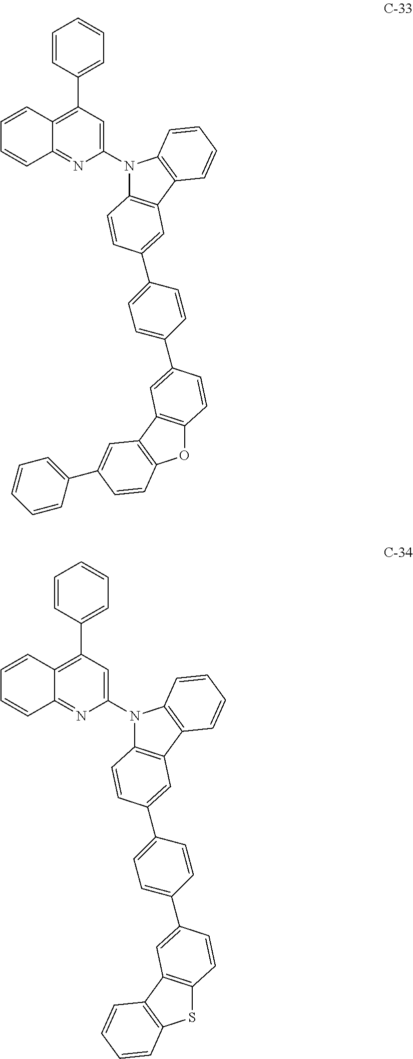 [SMILES: c1ccc(-c2ccc3oc4ccc(-c5ccc(-c6ccc7c(c6)c6ccccc6n7-c6cc(-c7ccccc7)c7ccccc7n6)cc5)cc4c3c2)cc1 ; c1ccc(-c2cc(-n3c4ccccc4c4cc(-c5ccc(-c6ccc7sc8ccccc8c7c6)cc5)ccc43)nc3ccccc23)cc1]

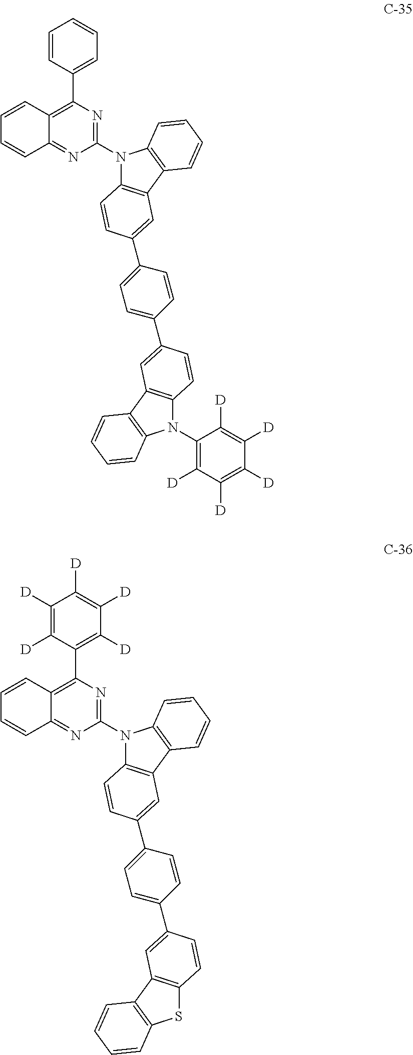 [SMILES: [2H]c1c([2H])c([2H])c(-n2c3ccccc3c3cc(-c4ccc(-c5ccc6c(c5)c5ccccc5n6-c5nc(-c6ccccc6)c6ccccc6n5)cc4)ccc32)c([2H])c1[2H] ; [2H]c1c([2H])c([2H])c(-c2nc(-n3c4ccccc4c4cc(-c5ccc(-c6ccc7sc8ccccc8c7c6)cc5)ccc43)nc3ccccc23)c([2H])c1[2H]]

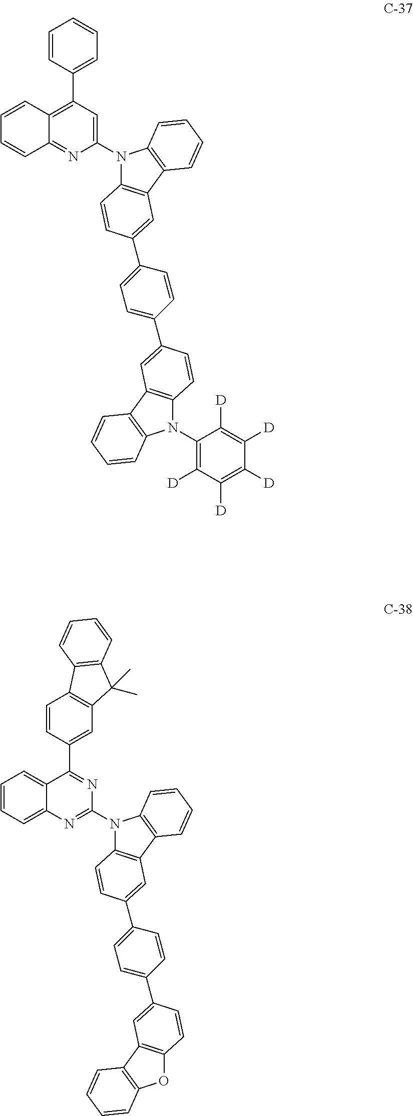 [SMILES: [2H]c1c([2H])c([2H])c(-n2c3ccccc3c3cc(-c4ccc(-c5ccc6c(c5)c5ccccc5n6-c5cc(-c6ccccc6)c6ccccc6n5)cc4)ccc32)c([2H])c1[2H] ; CC1(C)c2ccccc2-c2ccc(-c3nc(-n4c5ccccc5c5cc(-c6ccc(-c7ccc8oc9ccccc9c8c7)cc6)ccc54)nc4ccccc34)cc21]

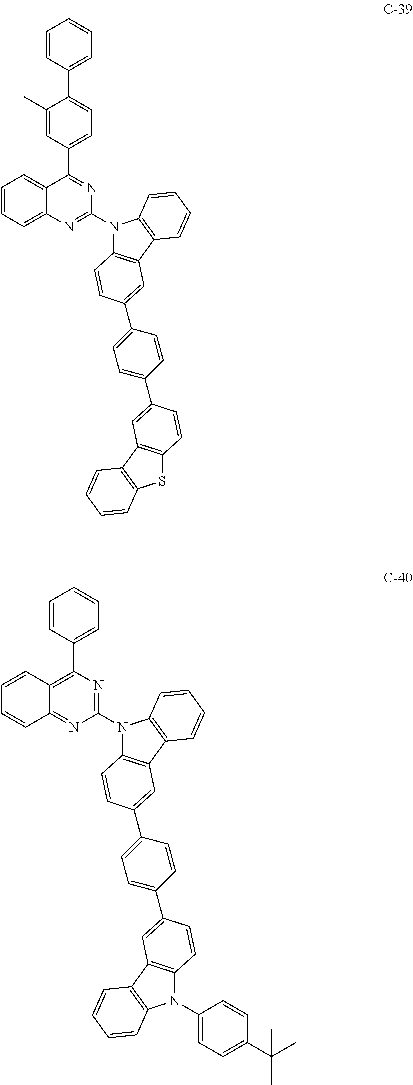 [SMILES: Cc1cc(-c2nc(-n3c4ccccc4c4cc(-c5ccc(-c6ccc7sc8ccccc8c7c6)cc5)ccc43)nc3ccccc23)ccc1-c1ccccc1 ; CC(C)(C)c1ccc(-n2c3ccccc3c3cc(-c4ccc(-c5ccc6c(c5)c5ccccc5n6-c5nc(-c6ccccc6)c6ccccc6n5)cc4)ccc32)cc1]

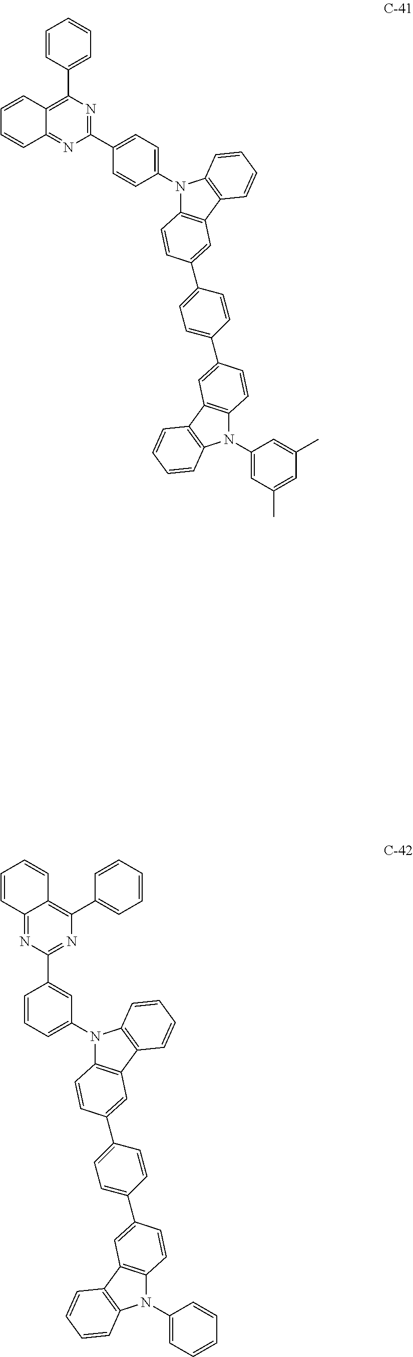 [SMILES: Cc1cc(C)cc(-n2c3ccccc3c3cc(-c4ccc(-c5ccc6c(c5)c5ccccc5n6-c5ccc(-c6nc(-c7ccccc7)c7ccccc7n6)cc5)cc4)ccc32)c1 ; c1ccc(-c2nc(-c3cccc(-n4c5ccccc5c5cc(-c6ccc(-c7ccc8c(c7)c7ccccc7n8-c7ccccc7)cc6)ccc54)c3)nc3ccccc23)cc1]

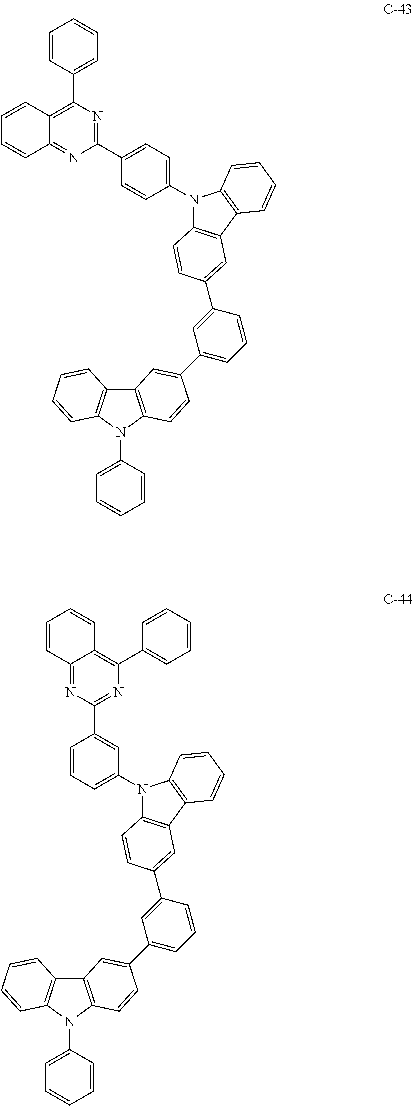 [SMILES: c1ccc(-c2nc(-c3ccc(-n4c5ccccc5c5cc(-c6cccc(-c7ccc8c(c7)c7ccccc7n8-c7ccccc7)c6)ccc54)cc3)nc3ccccc23)cc1 ; c1ccc(-c2nc(-c3cccc(-n4c5ccccc5c5cc(-c6cccc(-c7ccc8c(c7)c7ccccc7n8-c7ccccc7)c6)ccc54)c3)nc3ccccc23)cc1]

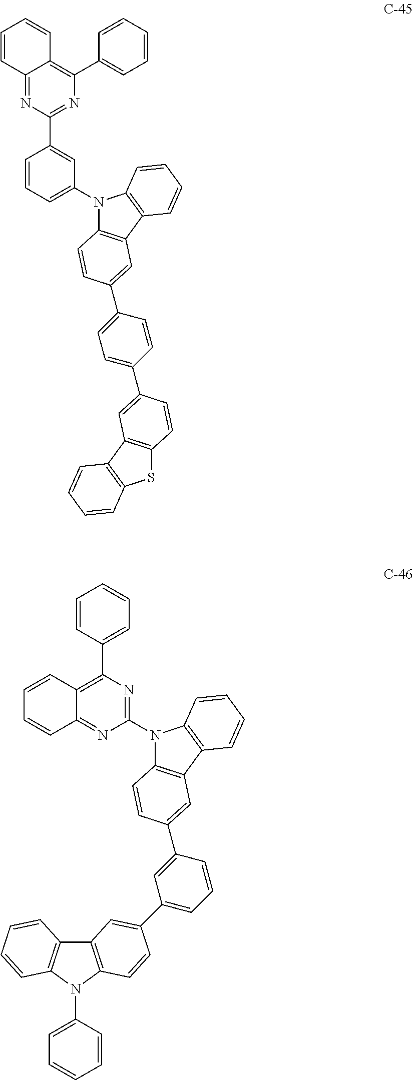 [SMILES: c1ccc(-c2nc(-c3cccc(-n4c5ccccc5c5cc(-c6ccc(-c7ccc8sc9ccccc9c8c7)cc6)ccc54)c3)nc3ccccc23)cc1 ; c1ccc(-c2nc(-n3c4ccccc4c4cc(-c5cccc(-c6ccc7c(c6)c6ccccc6n7-c6ccccc6)c5)ccc43)nc3ccccc23)cc1]

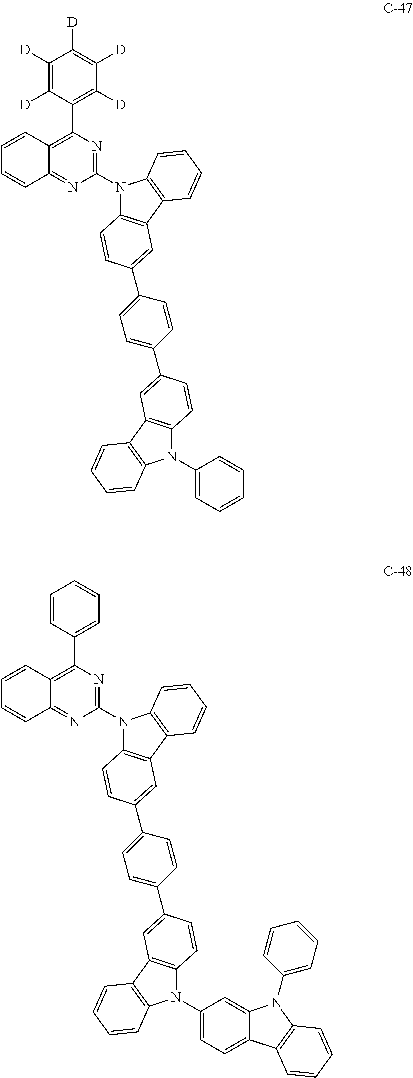 [SMILES: [2H]c1c([2H])c([2H])c(-c2nc(-n3c4ccccc4c4cc(-c5ccc(-c6ccc7c(c6)c6ccccc6n7-c6ccccc6)cc5)ccc43)nc3ccccc23)c([2H])c1[2H] ; c1ccc(-c2nc(-n3c4ccccc4c4cc(-c5ccc(-c6ccc7c(c6)c6ccccc6n7-c6ccc7c8ccccc8n(-c8ccccc8)c7c6)cc5)ccc43)nc3ccccc23)cc1]

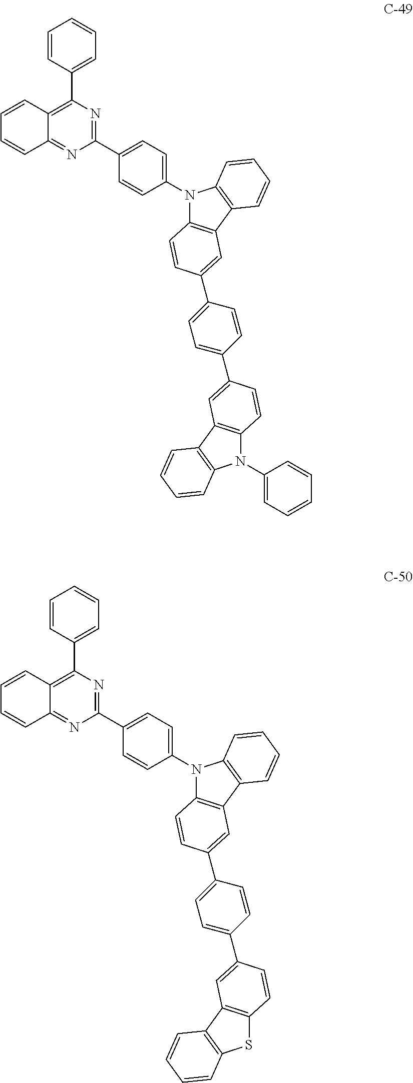 [SMILES: c1ccc(-c2nc(-c3ccc(-n4c5ccccc5c5cc(-c6ccc(-c7ccc8c(c7)c7ccccc7n8-c7ccccc7)cc6)ccc54)cc3)nc3ccccc23)cc1 ; c1ccc(-c2nc(-c3ccc(-n4c5ccccc5c5cc(-c6ccc(-c7ccc8sc9ccccc9c8c7)cc6)ccc54)cc3)nc3ccccc23)cc1]

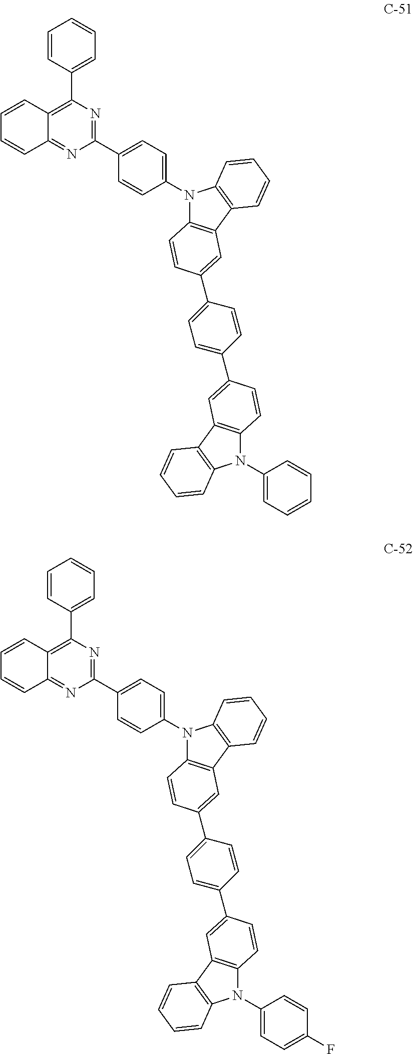 [SMILES: c1ccc(-c2nc(-c3ccc(-n4c5ccccc5c5cc(-c6ccc(-c7ccc8c(c7)c7ccccc7n8-c7ccccc7)cc6)ccc54)cc3)nc3ccccc23)cc1 ; Fc1ccc(-n2c3ccccc3c3cc(-c4ccc(-c5ccc6c(c5)c5ccccc5n6-c5ccc(-c6nc(-c7ccccc7)c7ccccc7n6)cc5)cc4)ccc32)cc1]

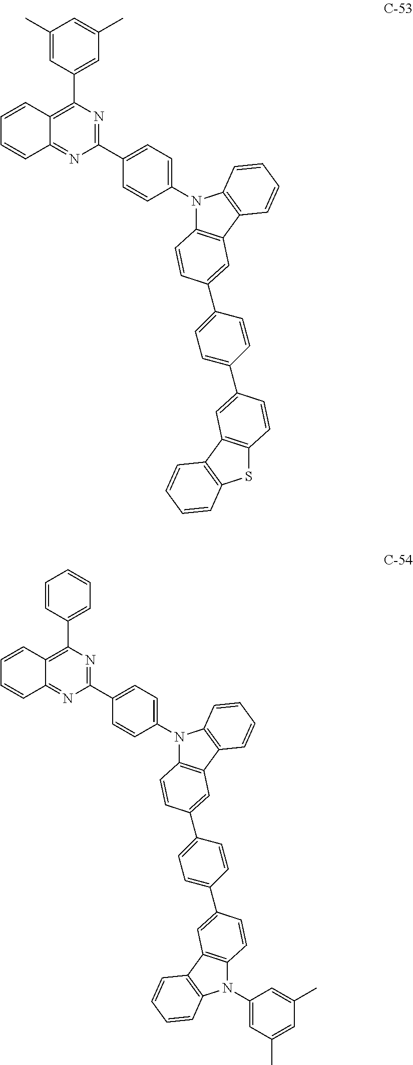 [SMILES: Cc1cc(C)cc(-c2nc(-c3ccc(-n4c5ccccc5c5cc(-c6ccc(-c7ccc8sc9ccccc9c8c7)cc6)ccc54)cc3)nc3ccccc23)c1 ; Cc1cc(C)cc(-n2c3ccccc3c3cc(-c4ccc(-c5ccc6c(c5)c5ccccc5n6-c5ccc(-c6nc(-c7ccccc7)c7ccccc7n6)cc5)cc4)ccc32)c1]

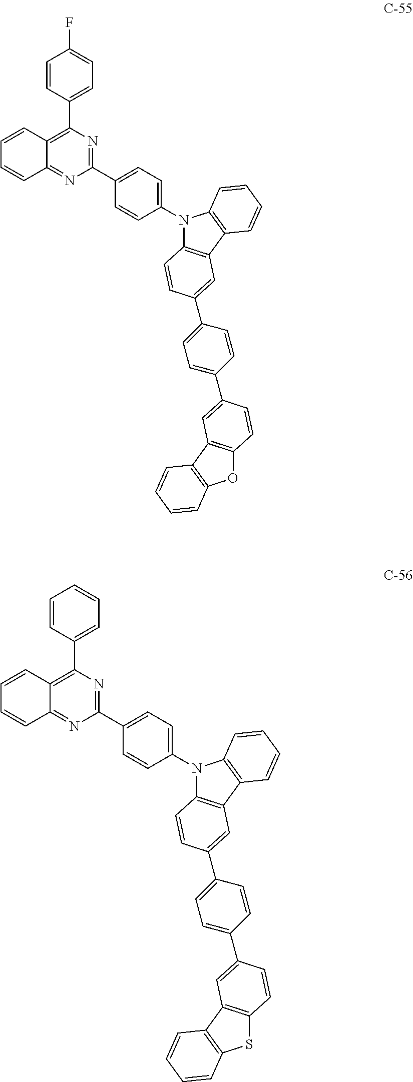 [SMILES: Fc1ccc(-c2nc(-c3ccc(-n4c5ccccc5c5cc(-c6ccc(-c7ccc8oc9ccccc9c8c7)cc6)ccc54)cc3)nc3ccccc23)cc1 ; c1ccc(-c2nc(-c3ccc(-n4c5ccccc5c5cc(-c6ccc(-c7ccc8sc9ccccc9c8c7)cc6)ccc54)cc3)nc3ccccc23)cc1]

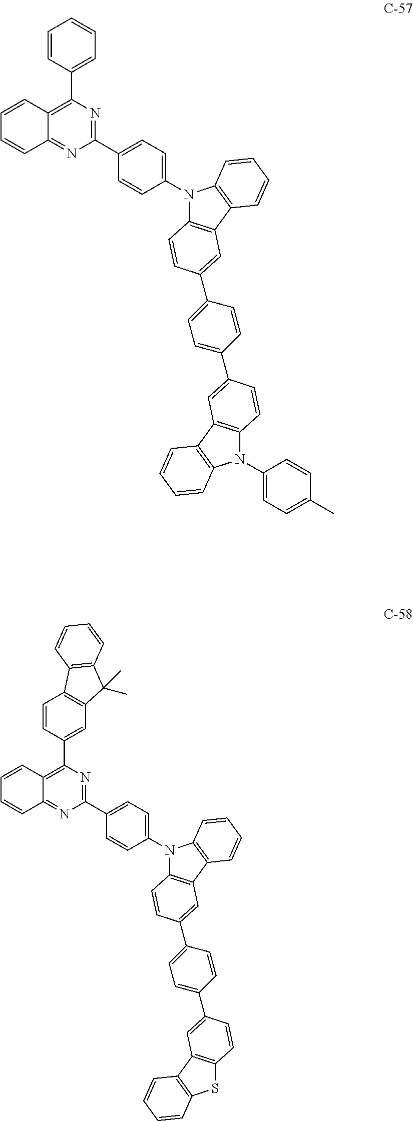 [SMILES: Cc1ccc(-n2c3ccccc3c3cc(-c4ccc(-c5ccc6c(c5)c5ccccc5n6-c5ccc(-c6nc(-c7ccccc7)c7ccccc7n6)cc5)cc4)ccc32)cc1 ; CC1(C)c2ccccc2-c2ccc(-c3nc(-c4ccc(-n5c6ccccc6c6cc(-c7ccc(-c8ccc9sc%10ccccc%10c9c8)cc7)ccc65)cc4)nc4ccccc34)cc21]

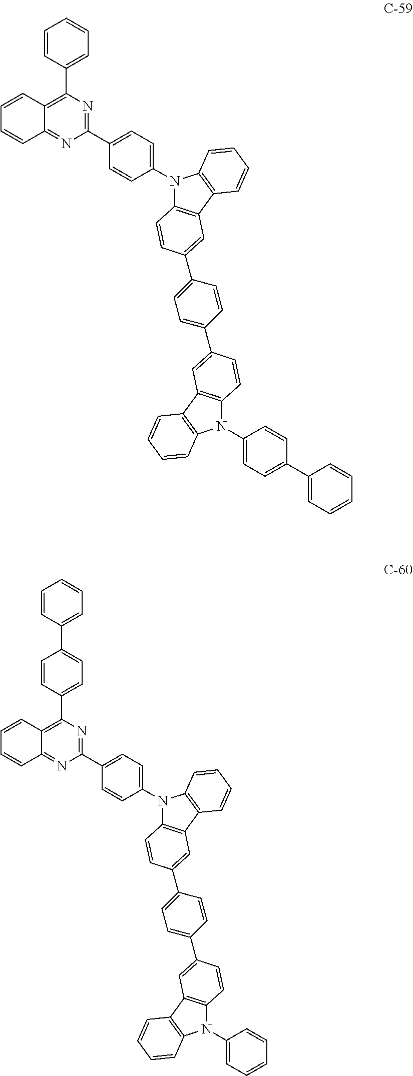 [SMILES: c1ccc(-c2ccc(-n3c4ccccc4c4cc(-c5ccc(-c6ccc7c(c6)c6ccccc6n7-c6ccc(-c7nc(-c8ccccc8)c8ccccc8n7)cc6)cc5)ccc43)cc2)cc1 ; c1ccc(-c2ccc(-c3nc(-c4ccc(-n5c6ccccc6c6cc(-c7ccc(-c8ccc9c(c8)c8ccccc8n9-c8ccccc8)cc7)ccc65)cc4)nc4ccccc34)cc2)cc1]

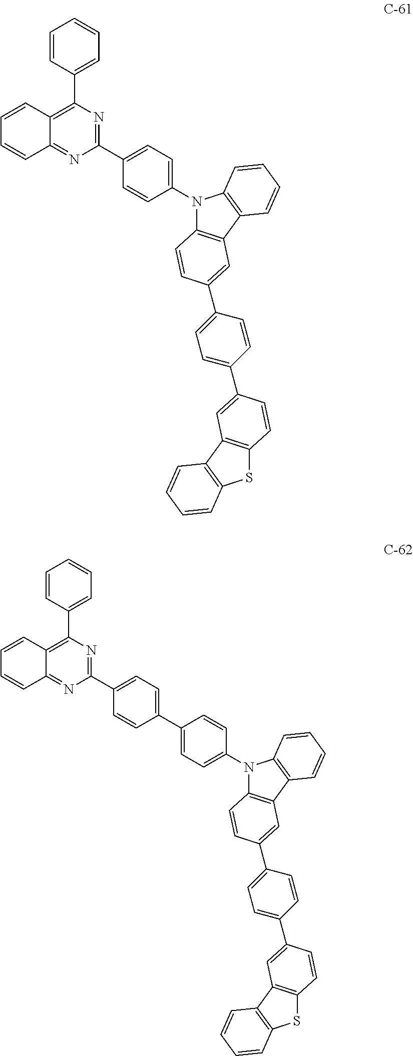 [SMILES: c1ccc(-c2nc(-c3ccc(-n4c5ccccc5c5cc(-c6ccc(-c7ccc8sc9ccccc9c8c7)cc6)ccc54)cc3)nc3ccccc23)cc1 ; c1ccc(-c2nc(-c3ccc(-c4ccc(-n5c6ccccc6c6cc(-c7ccc(-c8ccc9sc%10ccccc%10c9c8)cc7)ccc65)cc4)cc3)nc3ccccc23)cc1]

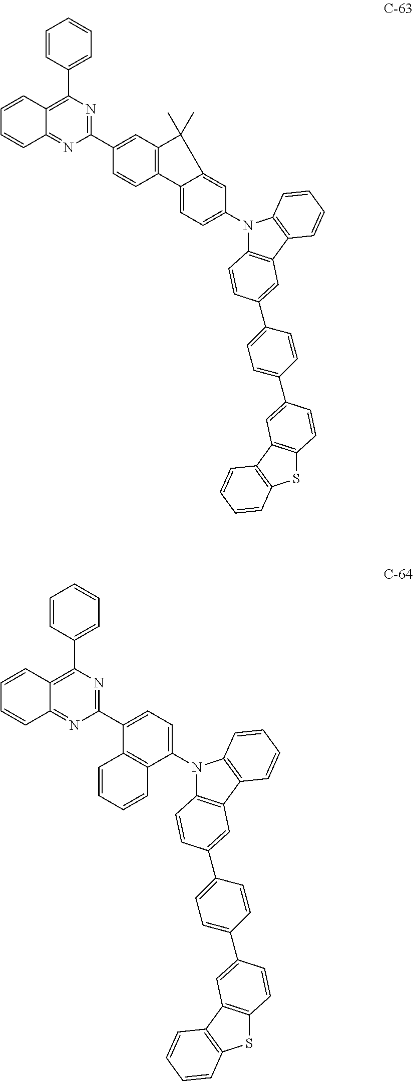 [SMILES: CC1(C)c2cc(-c3nc(-c4ccccc4)c4ccccc4n3)ccc2-c2ccc(-n3c4ccccc4c4cc(-c5ccc(-c6ccc7sc8ccccc8c7c6)cc5)ccc43)cc21 ; c1ccc(-c2nc(-c3ccc(-n4c5ccccc5c5cc(-c6ccc(-c7ccc8sc9ccccc9c8c7)cc6)ccc54)c4ccccc34)nc3ccccc23)cc1]

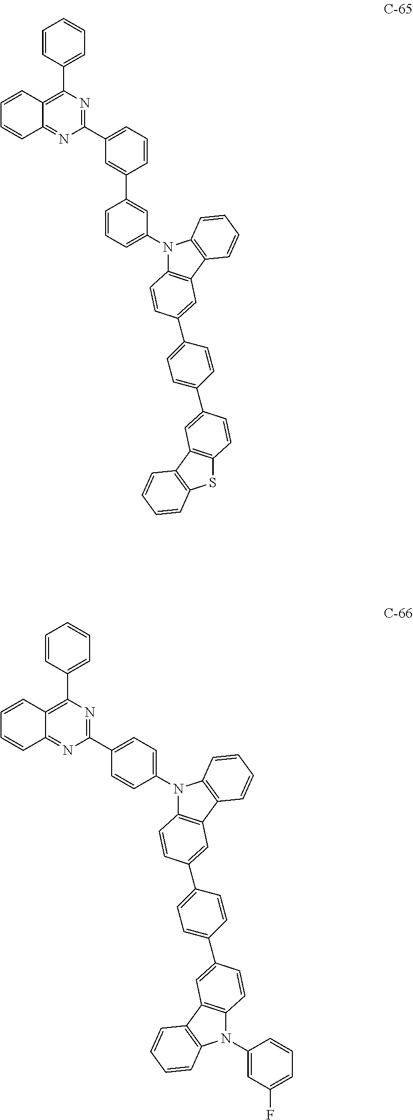 [SMILES: c1ccc(-c2nc(-c3cccc(-c4cccc(-n5c6ccccc6c6cc(-c7ccc(-c8ccc9sc%10ccccc%10c9c8)cc7)ccc65)c4)c3)nc3ccccc23)cc1 ; Fc1cccc(-n2c3ccccc3c3cc(-c4ccc(-c5ccc6c(c5)c5ccccc5n6-c5ccc(-c6nc(-c7ccccc7)c7ccccc7n6)cc5)cc4)ccc32)c1]

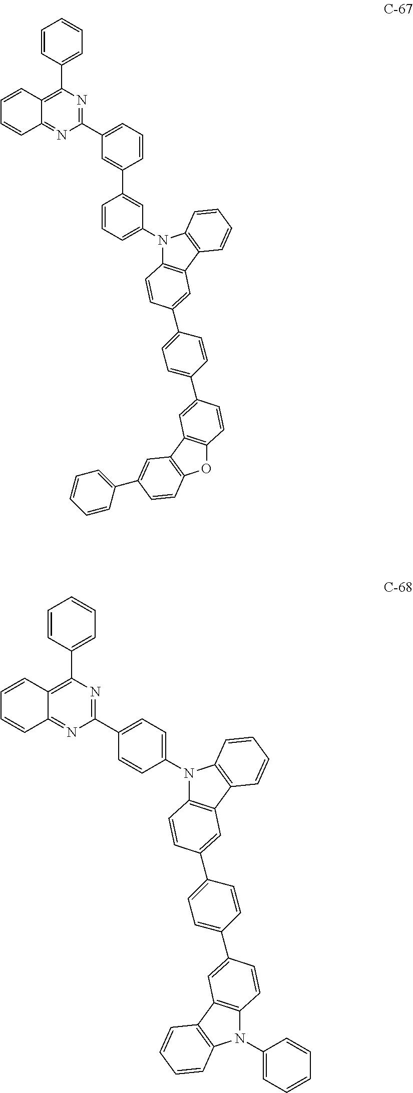 [SMILES: c1ccc(-c2ccc3oc4ccc(-c5ccc(-c6ccc7c(c6)c6ccccc6n7-c6cccc(-c7cccc(-c8nc(-c9ccccc9)c9ccccc9n8)c7)c6)cc5)cc4c3c2)cc1 ; c1ccc(-c2nc(-c3ccc(-n4c5ccccc5c5cc(-c6ccc(-c7ccc8c(c7)c7ccccc7n8-c7ccccc7)cc6)ccc54)cc3)nc3ccccc23)cc1]

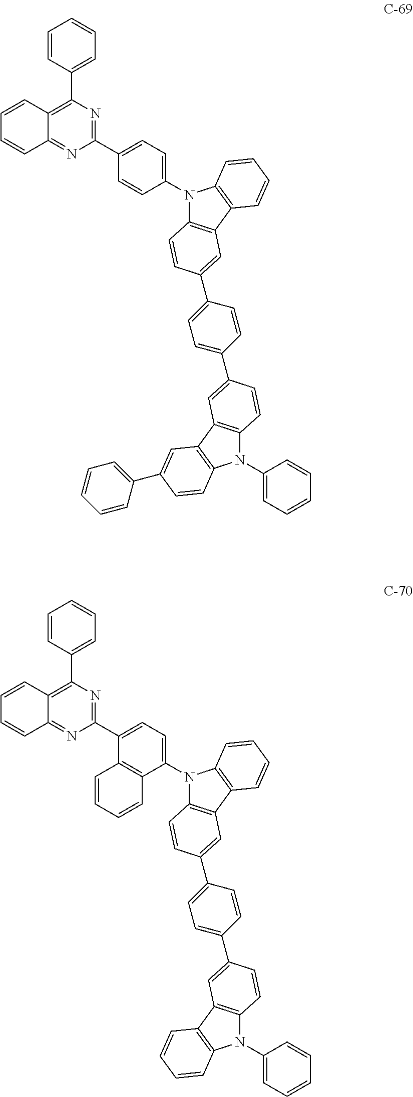 [SMILES: c1ccc(-c2ccc3c(c2)c2cc(-c4ccc(-c5ccc6c(c5)c5ccccc5n6-c5ccc(-c6nc(-c7ccccc7)c7ccccc7n6)cc5)cc4)ccc2n3-c2ccccc2)cc1 ; c1ccc(-c2nc(-c3ccc(-n4c5ccccc5c5cc(-c6ccc(-c7ccc8c(c7)c7ccccc7n8-c7ccccc7)cc6)ccc54)c4ccccc34)nc3ccccc23)cc1]

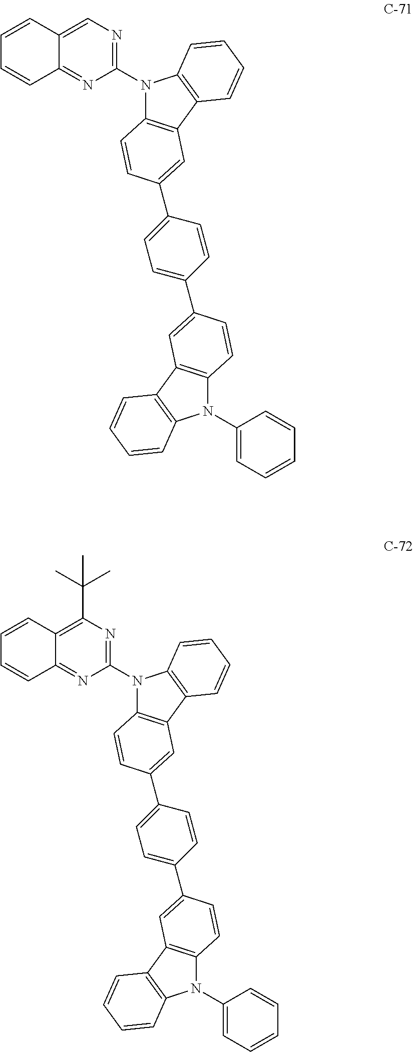 [SMILES: c1ccc(-n2c3ccccc3c3cc(-c4ccc(-c5ccc6c(c5)c5ccccc5n6-c5ncc6ccccc6n5)cc4)ccc32)cc1 ; CC(C)(C)c1nc(-n2c3ccccc3c3cc(-c4ccc(-c5ccc6c(c5)c5ccccc5n6-c5ccccc5)cc4)ccc32)nc2ccccc12]

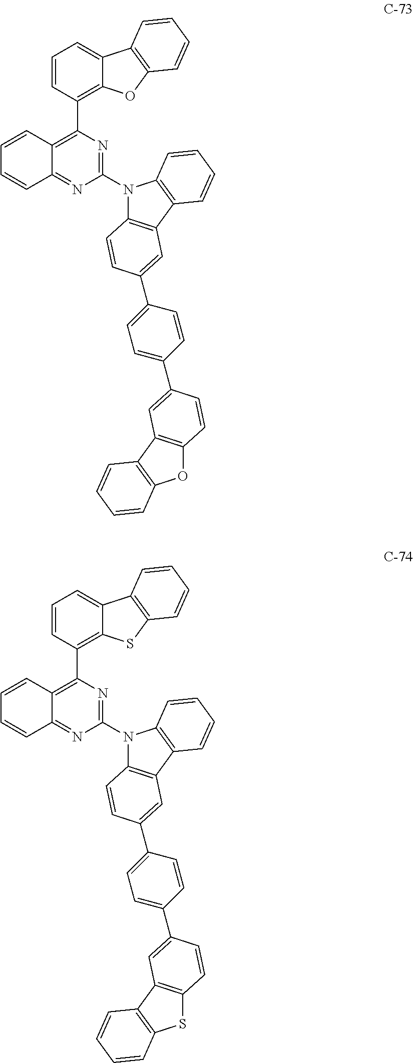 [SMILES: c1ccc2c(-c3cccc4c3oc3ccccc34)nc(-n3c4ccccc4c4cc(-c5ccc(-c6ccc7oc8ccccc8c7c6)cc5)ccc43)nc2c1 ; c1ccc2c(-c3cccc4c3sc3ccccc34)nc(-n3c4ccccc4c4cc(-c5ccc(-c6ccc7sc8ccccc8c7c6)cc5)ccc43)nc2c1]

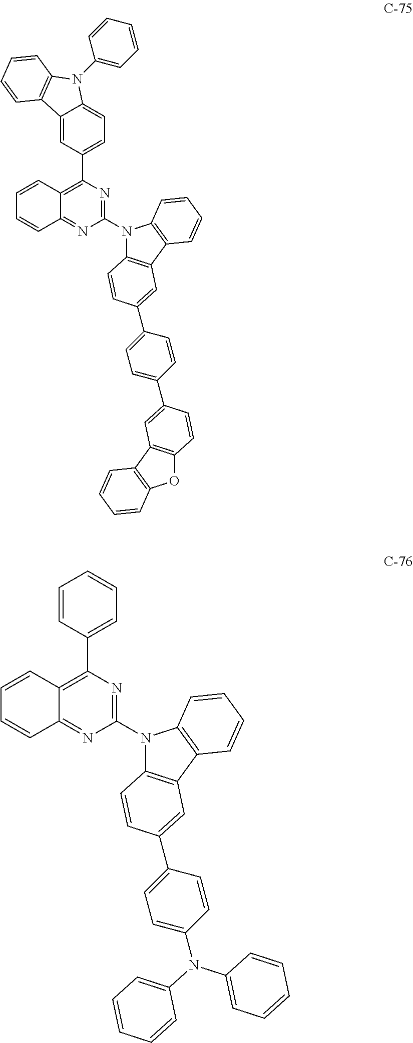 [SMILES: c1ccc(-n2c3ccccc3c3cc(-c4nc(-n5c6ccccc6c6cc(-c7ccc(-c8ccc9oc%10ccccc%10c9c8)cc7)ccc65)nc5ccccc45)ccc32)cc1 ; c1ccc(-c2nc(-n3c4ccccc4c4cc(-c5ccc(N(c6ccccc6)c6ccccc6)cc5)ccc43)nc3ccccc23)cc1]

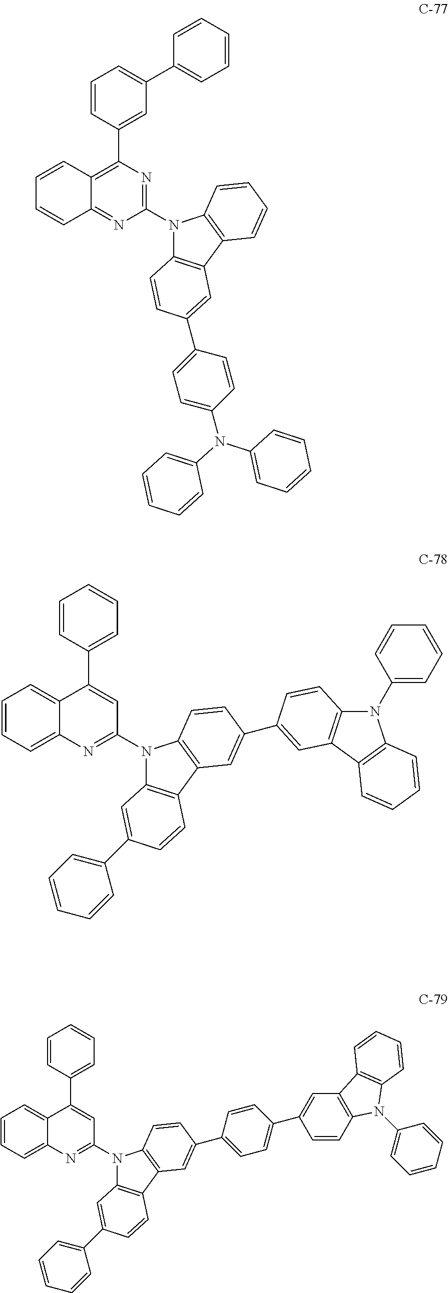 [SMILES: c1ccc(-c2cccc(-c3nc(-n4c5ccccc5c5cc(-c6ccc(N(c7ccccc7)c7ccccc7)cc6)ccc54)nc4ccccc34)c2)cc1 ; c1ccc(-c2ccc3c4cc(-c5ccc6c(c5)c5ccccc5n6-c5ccccc5)ccc4n(-c4cc(-c5ccccc5)c5ccccc5n4)c3c2)cc1 ; c1ccc(-c2ccc3c4cc(-c5ccc(-c6ccc7c(c6)c6ccccc6n7-c6ccccc6)cc5)ccc4n(-c4cc(-c5ccccc5)c5ccccc5n4)c3c2)cc1]

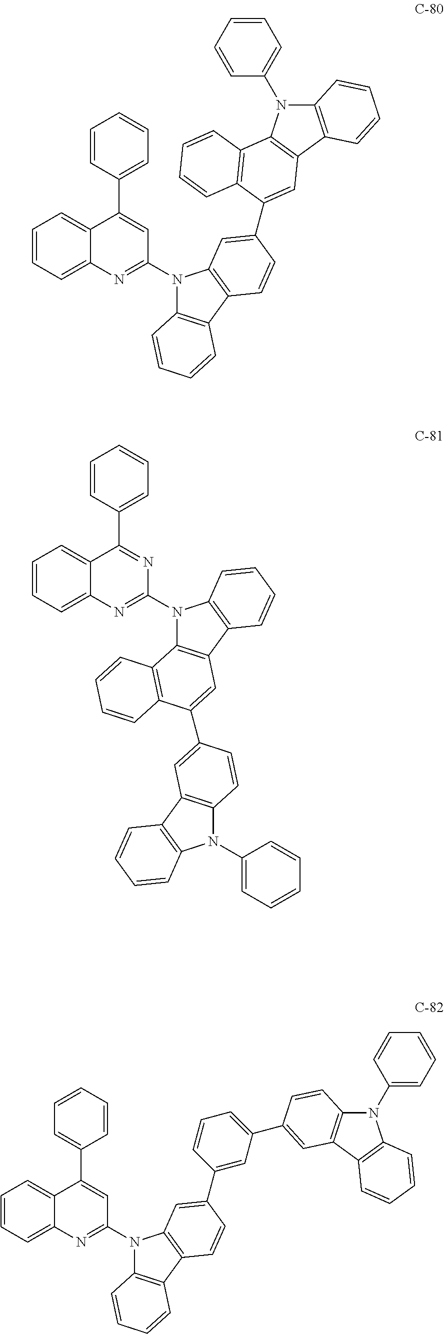 [SMILES: c1ccc(-c2cc(-n3c4ccccc4c4ccc(-c5cc6c7ccccc7n(-c7ccccc7)c6c6ccccc56)cc43)nc3ccccc23)cc1 ; c1ccc(-c2nc(-n3c4ccccc4c4cc(-c5ccc6c(c5)c5ccccc5n6-c5ccccc5)c5ccccc5c43)nc3ccccc23)cc1 ; c1ccc(-c2cc(-n3c4ccccc4c4ccc(-c5cccc(-c6ccc7c(c6)c6ccccc6n7-c6ccccc6)c5)cc43)nc3ccccc23)cc1]

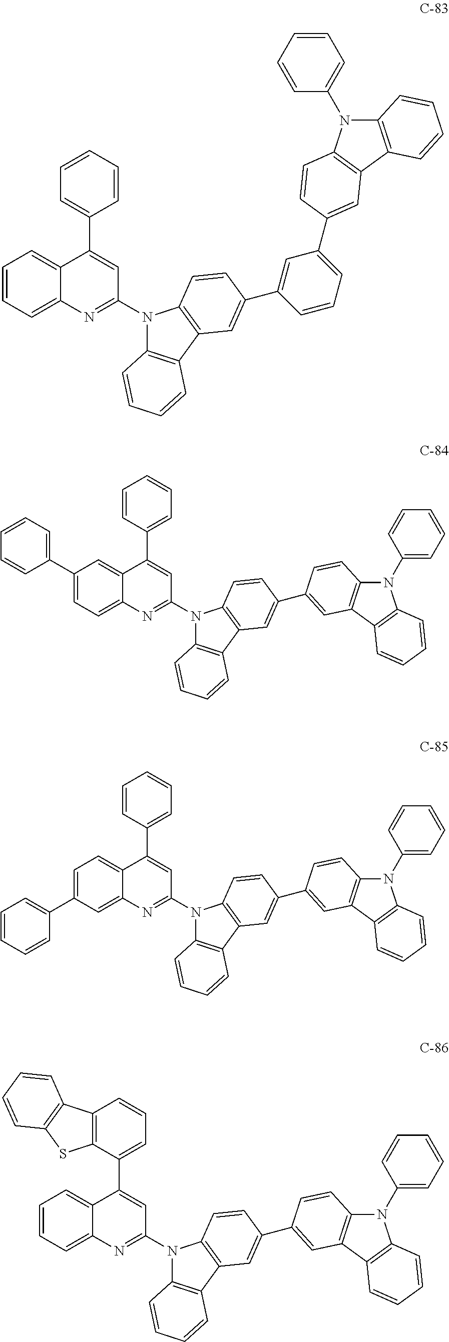 [SMILES: c1ccc(-c2cc(-n3c4ccccc4c4cc(-c5cccc(-c6ccc7c(c6)c6ccccc6n7-c6ccccc6)c5)ccc43)nc3ccccc23)cc1 ; c1ccc(-c2ccc3nc(-n4c5ccccc5c5cc(-c6ccc7c(c6)c6ccccc6n7-c6ccccc6)ccc54)cc(-c4ccccc4)c3c2)cc1 ; c1ccc(-c2ccc3c(-c4ccccc4)cc(-n4c5ccccc5c5cc(-c6ccc7c(c6)c6ccccc6n7-c6ccccc6)ccc54)nc3c2)cc1 ; c1ccc(-n2c3ccccc3c3cc(-c4ccc5c(c4)c4ccccc4n5-c4cc(-c5cccc6c5sc5ccccc56)c5ccccc5n4)ccc32)cc1]

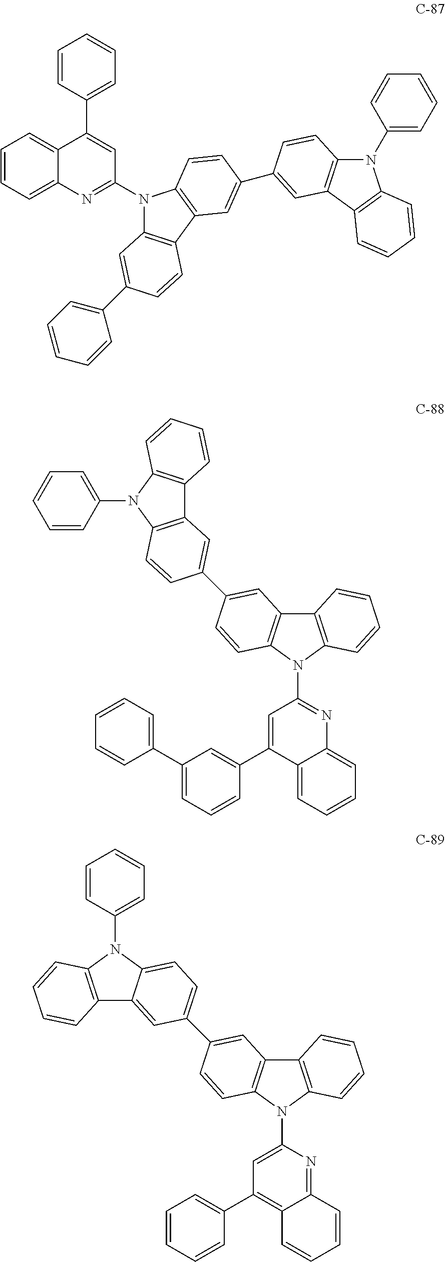 [SMILES: c1ccc(-c2ccc3c4cc(-c5ccc6c(c5)c5ccccc5n6-c5ccccc5)ccc4n(-c4cc(-c5ccccc5)c5ccccc5n4)c3c2)cc1 ; c1ccc(-c2cccc(-c3cc(-n4c5ccccc5c5cc(-c6ccc7c(c6)c6ccccc6n7-c6ccccc6)ccc54)nc4ccccc34)c2)cc1 ; c1ccc(-c2cc(-n3c4ccccc4c4cc(-c5ccc6c(c5)c5ccccc5n6-c5ccccc5)ccc43)nc3ccccc23)cc1]

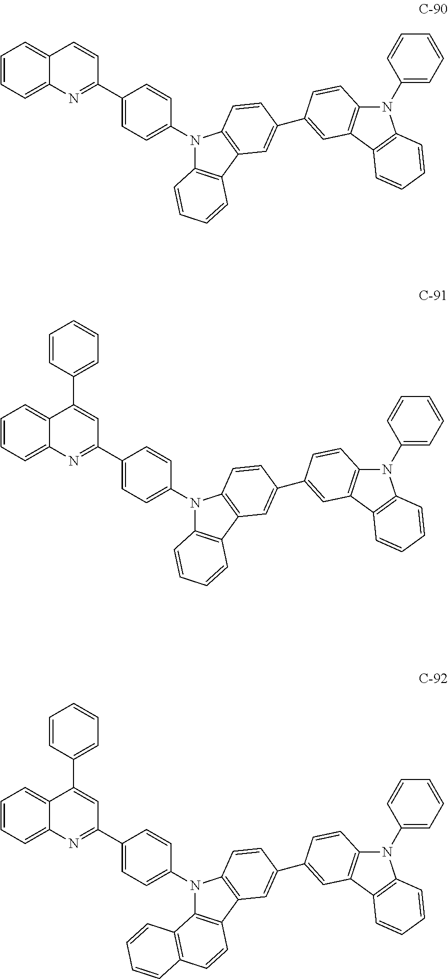 [SMILES: c1ccc(-n2c3ccccc3c3cc(-c4ccc5c(c4)c4ccccc4n5-c4ccc(-c5ccc6ccccc6n5)cc4)ccc32)cc1 ; c1ccc(-c2cc(-c3ccc(-n4c5ccccc5c5cc(-c6ccc7c(c6)c6ccccc6n7-c6ccccc6)ccc54)cc3)nc3ccccc23)cc1 ; c1ccc(-c2cc(-c3ccc(-n4c5ccc(-c6ccc7c(c6)c6ccccc6n7-c6ccccc6)cc5c5ccc6ccccc6c54)cc3)nc3ccccc23)cc1]

6. An organic electroluminescent device comprising the organic electroluminescent compound according to claim 1.
